(19) United States
(12) Patent Application Publication
(10) Pub. No.: US 2025/0067743 A1
(43) Pub. Date: Feb. 27, 2025

(54) PREDICTIVE AND DIAGNOSTIC SCREENING METHODS FOR ENDOMETRIAL CANCER

(71) Applicant: ARIZONA BOARD OF REGENTS ON BEHALF OF THE UNIVERSITY OF ARIZONA, Tucson, AZ (US)

(72) Inventors: Melissa M. Herbst-Kralovetz, Phoenix, AZ (US); Pawel Laniewski, Phoenix, AZ (US)

(21) Appl. No.: 18/943,508

(22) Filed: Nov. 11, 2024

Related U.S. Application Data
(63) Continuation-in-part of application No. PCT/US23/66947, filed on May 12, 2023.
(60) Provisional application No. 63/341,171, filed on May 12, 2022.

(51) Int. Cl. G01N 33/574

(52) U.S. Cl. CPC G01N 33/57442; G01N 33/57488

(57) ABSTRACT

Endometrial cancer (EC) is the most common gynecologic cancer in developed countries and the fourth most common cancer affecting women in the US. In contrast to other cancers, rates of EC continue to rise, and there are indications that social determinants of health and race and/or ethnicity contribute to risk. Thus, the methods described herein provide a non-invasive means of measuring protein biomarkers in the local cervicovaginal microenvironment. These novel biomarkers may be used for diagnosing and/or predicting women that are “at risk” for the development and progression of endometrial cancer (EC; e.g., EC type 1). Specifically, the methods herein may include obtaining a sample (e.g., CVL, vaginal swabs, or secretions) from a patient and producing a profile of at least five or more protein biomarkers from the collected sample.

### CROSS-REFERENCES TO RELATED APPLICATIONS

This application is a Continuation-in-Part and claims benefit of PCT Application No. PCT/US23/66947 filed May 12, 2023, which claims benefit of U.S. Provisional Patent Application No. 63/341,171 filed May 12, 2022, the specification of which is incorporated herein its entirety by reference.

### FIELD OF THE INVENTION

The present invention relates to methods for predictive and diagnostic screening of women at risk for the development and progression of endometrial cancer. The methods feature detecting particular biomarkers using the local microenvironment.

### BACKGROUND OF THE INVENTION

Endometrial cancer (EC) is the most common gynecologic cancer and the fourth most common cancer affecting women in high-income countries. In contrast to other malignancies, rates of EC continue to rise. The International Agency for Research on Cancer estimates that EC rates will increase by more than 50% worldwide by 2040. EC risk factors include increased age, higher BMI, metabolic syndrome, estrogen exposure, tamoxifen use, early menarche, late menopause, lower parity, and genetic predisposition. There are also indications that social determinants of health and race/ethnicity contribute to risk and survival rates. For example, in the USA, Black women with EC have an overall 55% higher 5-year mortality risk compared to White women, likely due to delayed diagnosis. Hispanic and Native American women also have higher incidence and poorer survival rates of EC than non-Hispanic White women. In Europe, a Swedish study revealed that women with lower socioeconomic status are generally diagnosed at the late cancer stage and have reduced survival compared to women with higher socioeconomic status.

Historically, EC was grouped into two categories: type I (most common, estrogen-driven, composed of grade 1 or 2 endometrioid carcinomas with a favorable prognosis) and type II (less common, composed of high-grade endometrioid carcinomas or other non-endometrioid subtypes, more aggressive with a poor prognosis). Yet, EC is heterogeneous at the molecular level. The new classification of EC into four molecular subgroups has been identified by The Cancer Genome Atlas Project. These subgroups were defined by mutation burden and copy number alterations, including microsatellite instability with mismatch repair (MMR) defect, hypermutation of POLE gene, extensive genomic amplifications/deletions (copy number high), and low amount of genomic alterations (copy number low). Importantly, this and other molecular classifications allow subdividing EC into distinct prognostic groups, thus helping determine treatment options and improve clinical outcomes.

EC is most often diagnosed in symptomatic women with abnormal uterine bleeding; however, this symptom is also common for other gynecologic conditions. Currently, the gold standard for diagnosing EC is endometrial biopsy with or without hysteroscopy or dilation and curettage, which involves dilation of the cervix and scraping of the endometrial lining. Although these surgical procedures are considered to be minimally invasive and generally safe, they still carry risks of complications, including uterine perforation, uterine infection, and hemorrhage. In addition, current sampling methods for EC diagnosis can cause anxiety, physical discomfort and/or pain, which impact acceptability and accessibility. Thus, there is a need to develop a non-invasive and low-cost method for early EC detection.

Proteins are easily detectable and quantifiable in a variety of biological fluids, therefore, commonly tested as potential biomarkers for cancer detection. For EC, protein biomarkers have been mainly tested in blood or tissue samples. The two most studied proteins included human epididymis protein (HE4) and cancer antigen (CA) 125, both elevated in endometrial tissues and serum of EC patients. Although specific, these biomarkers (analyzed individually or in combination) failed to demonstrate high sensitivity. Thus, additional research is needed to quantify protein biomarkers in the context of EC for sufficient diagnostic accuracy, preferably using samples collected by a non-invasive method.

### BRIEF SUMMARY OF THE INVENTION

It is an objective of the present invention to provide methods that allow for non-invasive point-of-care testing for early diagnosis of endometrial hyperplasia and cancer as specified in the independent claims. Embodiments of the invention are given in the dependent claims. Embodiments of the present invention can be freely combined with each other if they are not mutually exclusive.

Typically, EC is diagnosed in peri- and postmenopausal women with abnormal uterine bleeding. Although this symptom is prevalent in EC patients (occurs in approximately 90% of cases), only 9% of women with abnormal uterine bleeding are actually diagnosed with EC. For a definitive diagnosis of EC, symptomatic women undergo various painful, anxiety-provoking, and time-consuming medical procedures, such as hysteroscopy, endometrial biopsy, and dilation and curettage. This diagnostic approach forms a barrier to early detection and treatment, particularly in populations with limited or inadequate access to healthcare. Thus, novel diagnostic methods, ideally based on non-to minimally invasive sampling, are needed to improve detection, increase acceptability, and ultimately reduce morbidity and mortality related to this common gynecological cancer.

Herein, the present invention features a novel approach for detecting EC using lavage sampling of the cervicovaginal microenvironment coupled with multiplex immunoassay technology. The present invention features novel biomarkers with high predictive accuracy and sensitivity/specificity for early EC diagnosis. The diagnostic biomarker levels in cervicovaginal lavage (CVL) will be altered in hyperplasia and EC relative to benign conditions.

In some embodiments, the present invention features a method comprising obtaining a biological sample (e.g., cervicovaginal lavage (CVL) sample) from a patient, producing a profile of the biological sample (e.g., CVL sample) collected in the preceding step by detecting at least five or more protein biomarkers and analyzing the biological sample (CVL sample) profile produced in the preceding step. In some embodiments, the protein biomarkers are cervicovaginal protein biomarkers.

In other embodiments, the present invention features a method comprising a) obtaining a biological sample (e.g., CVL sample) from a patient, b) producing a profile of the biological sample (e.g., CVL sample) collected in (a) by detecting at least five protein biomarkers selected from the group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3), and c) analyzing the biological sample (e.g., CVL sample) profile produced in (b). In some embodiments, the method comprising producing a profile of the biological sample (e.g., CVL sample) collected in (a) by detecting at least ten protein biomarkers.

In other embodiments, the present invention features a method of diagnosing endometrial cancer (EC) in a subject in need thereof. The method may comprise a) obtaining a cervicovaginal lavage (CVL) sample from the subject, b) producing a profile of the CVL sample collected by detecting at least five or more protein biomarkers, and c) analyzing the CVL sample profile produced. In some embodiments, the subject is diagnosed with EC if the levels of at least five biomarkers are altered compared to a healthy control profile. The present invention may also feature methods of treating endometrial cancer (EC) in a subject in need thereof, where if a subject is diagnosed with EC, then an EC treatment is administered to the subject.

In other embodiments, the method of diagnosing endometrial cancer (EC) in a subject in need thereof comprising: a) obtaining a biological sample (e.g., CVL sample) from the subject, b) producing a profile of the biological sample collected in (a) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3), and c) analyzing the biological sample (e.g., CVL sample) profile produced in (b). In some embodiments, the method comprising producing a profile of the biological sample (e.g., CVL sample) collected in (a) by detecting at least ten protein biomarkers. In some embodiments, the subject is diagnosed with EC if the levels of at least five or ten biomarkers are altered compared to a healthy control profile. For example, the subject may be diagnosed with EC if the levels of angiopoietin-2, endoglin, FAP, ferritin, FGF-1, MIA, HB-EGF, or VEGF-A are elevated compared to the healthy control and the levels of galectin-3, MPO, or IGFBP-3 are decrease compared to the healthy control profile. The present invention may also feature methods of treating endometrial cancer (EC) in a subject in need thereof, where if a subject is diagnosed with EC, then an EC treatment is administered to the subject.

In further embodiments, the present invention features a method of monitoring a treatment for endometrial cancer (EC) in a subject in need thereof. The method may comprise obtaining a first biological sample (e.g., CVL sample) from the subject, producing a baseline profile of the biological sample collected by detecting at least five or more protein biomarkers and administering the treatment for EC to the subject. The method may further comprise obtaining a second biological sample (e.g., CVL sample) from the subject, producing a second profile of the second biological sample collected by detecting at least five or more protein biomarkers and comparing the baseline profile of the first biological sample to the second profile of the second biological sample. In some embodiments, the treatment is effective if the levels of at least five biomarkers are altered from the baseline profile as compared to the second profile, e.g., the treatment is effective if the levels of at least five biomarkers are decreased from the baseline profile as compared to the second profile.

In some embodiments, the method for monitoring a treatment for endometrial cancer (EC) in a subject in need thereof comprises a) obtaining a first biological sample from the subject; b) producing a baseline profile of the first biological sample collected in (a) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3); c) administering the treatment for EC to the subject; d) obtaining a second biological sample from the subject; e) producing a second profile of the second biological sample collected in (d) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3) and f) comparing the baseline profile of the first biological sample produced in (b) to the second profile of the second biological sample produced in (e). In some embodiments, the treatment is effective if the levels of at least five biomarkers are altered from the baseline profile as compared to the second profile. In other embodiments, the treatment is effective if the levels of at least ten biomarkers are altered from the baseline profile as compared to the second profile. For example, a treatment is effective if the levels of angiopoietin-2, endoglin, FAP, ferritin, FGF-1, MIA, HB-EGF, or VEGF-A are reduced from the baseline profile as compared to the second profile and/or the levels of galectin-3, MPO, or IGFBP-3 are elevated from the baseline profile as compared to the second profile.

In some embodiments, the methods described herein are in vitro and are not carried out directly on the subject.

One of the unique and inventive technical features of the present invention is non-invasive sampling (e.g., a cervicovaginal lavage (CVL)). Without wishing to limit the invention to any theory or mechanism, it is believed that the technical feature of the present invention advantageously provides for the detection of EC-related protein biomarkers in the cervicovaginal microenvironment. None of the presently known prior references or work has the unique, inventive technical feature of the present invention.

Furthermore, the prior references teach away from the present invention. For example, for a definitive diagnosis, women undergo various time-consuming and painful medical procedures, such as endometrial biopsy with or without hysteroscopy, and dilation and curettage, which may create a barrier to early detection and treatment, particularly for women with inadequate healthcare access. Specifically, invasive approaches create a barrier to screening, and there is currently no screening method for the early detection of EC in asymptomatic women.

Furthermore, the inventive technical features of the present invention contributed to a surprising result. For example, the targets that were most predictive were not the targets that were anticipated or predicted would be most predictive of disease status. Additional multivariate biomarker discovery analysis also yielded a unique set of targets that, when combined, were most predictive of disease status.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, and 1C show women diagnosed with EC exhibit distinct cervicovaginal protein profiles compared to women with benign conditions. A principal component analysis (PCA) of 72 proteins in cervicovaginal lavages (n=200) was displayed along the first two principal components (PC). Each point represents a single sample colored based on disease group (FIG. 1A), menopausal status (FIG. 1B), or body mass index (BMI) (FIG. 1C). Significant differences among the disease groups and between pre-and postmenopausal women were assessed using a multivariate analysis of variance (MANOVA) model.

FIGS. 2A, 2B, 2C, and 2D show cervicovaginal protein levels associated with the disease groups and menopausal status. FIG. 2A shows a heatmap that reflects relative levels of proteins in cervicovaginal lavages (CVL) across all the samples (n=192). Data were mean-centered and variance scaled along each row before clustering. Hierarchical clustering was based on Euclidean distance and Ward linkage. The analysis revealed two distinct clusters. Pie charts show the distribution of the disease groups (FIG. 2B), menopausal status (FIG. 2C), and BMI categories (FIG. 2D) were significantly different between the clusters. BMI categories did not vary between the clusters. P values were calculated using Fisher's exact test or chi-square test.

FIGS. 3A, 3B, 3C, 3D, 3E, 3F, and 3G show protein biomarkers in cervicovaginal lavages discriminate between patients diagnosed with EC from patients with benign conditions. Cervicovaginal biomarkers discriminating low-grade endometrial endometrioid carcinoma (EEC) or other endometrial cancer (EC) subtypes from benign conditions were identified using the receiver operating characteristics (ROC) analysis. The area under the curve (AUC) was reported for each tested protein, including cytokines (FIG. 3A), chemokines (FIG. 3B), growth factors (FIG. 3C), apoptosis-related proteins (FIG. 3D), hormones (FIG. 3E), tumor markers (FIG. 3F), and immune checkpoint proteins (FIG. 3G). The strength of the discriminators was measured with AUC values. Proteins with AUC greater than or equal to 0.8 or 0.9 (indicated with a square or diamond) were considered good or excellent discriminators, respectively.

FIGS. 4A and 4B show key cervicovaginal biomarkers are elevated in both low-grade endometrioid carcinoma and other endometrial subtypes. Cervicovaginal levels of proteins identified as good biomarkers for both low-grade EEC and other EC subtypes (FIG. 4A) or just for other EC subtypes (FIG. 4B) in the ROC analysis. Scatter dot plots show concentrations of these proteins in individual samples among the disease groups. A horizontal line indicates the mean. The significant differences were assessed using linear mixed-effects models with Bonferroni adjustment. Asterisks indicate P values adjusted (* P<0.05; ** P<0.01; *** P<0.001; *** P<0.0001).

FIGS. 5A, 5B, 5C, and 5D show the logistic regression model accurately predicts EC and benign conditions using protein biomarkers in CVL samples. The least absolute shrinkage and selection operator (LASSO) was performed to select features to build the logistic regression model (FIG. 5A). Twelve proteins with 100% frequency of LASSO selection were used to build the model. The performance of the model was evaluated using the Monte Carlo cross-validation. A multivariate ROC analysis, showing true and false positive rates, indicates excellent prediction of EC when compared to benign conditions (AUC 0.91) (FIG. 5B). A scatter plot depicts the predicted class probabilities of all samples using the classifier at a threshold of 0.5 (FIG. 5C). The confusion matrix illustrates the proportion of times each sample receives the correct classification (FIG. 5D). The logistic regression model correctly classified 151 out of 174 tested samples (86.8%).

FIGS. 6A and 6B show cervicovaginal levels of protein biomarkers in patients with EC vary based on histological type, MMR status, tumor size, and myometrial invasion. A volcano plot analysis was used to assess differences in the protein levels among patients with EC stratified based on tumor characteristics, including histological type and grade, mismatch repair (MMR) protein expression, tumor size, and presence of myometrial invasion (FIG. 6A). Statistical significance was determined using multiple t-tests with the false discovery rate (FDR) correction. A volcano plot indicates log2 differences (x-axis) and −log₁₀ (q value) (y-axis). Proteins with q<0.01 were considered significant. A correlation analysis between cervicovaginal levels of 72 proteins with the tumor size (measured in cm) and the depth of myometrial invasion (measured in mm) (FIG. 6B). Correlation coefficients (r) were calculated using the Spearman's rank correlation and depicted as a heatmap. P values are indicated with black circles. The biomarkers identified to be significant in both volcano and correlation analyses are marked with an asterisk (*).

FIGS. 7A, 7B, and 7C show differences in the first two principal components (PC1 and PC2) among the disease groups, menopausal status, and BMI categories. A principal component analysis (PCA) was performed using concentrations of 72 proteins in cervicovaginal lavages (n=192). The significant differences in PC1 and PC2 among the disease groups (FIG. 7A), menopausal status (FIG. 7B), and BMI categories (FIG. 7C) were assessed using an analysis of variance (ANOVA) with Tukey adjustment or unpaired two-tailed t-test. Asterisks indicate P values (* P<0.05; ** P<0.01; **** P<0.0001).

FIGS. 8A, 8B, 8C, and 8D show data on tumor size and depth of myometrial invasion for endometrial cancer patients. Data for low-grade endometrioid carcinoma (EEC) and other endometrial cancer (EC) types were extracted from pathology reports. Data on tumor size were available for 62 out of 66 patients diagnosed with EC. Data on the presence or absence of myometrial invasion were available for all EC patients (n=66). Data on the depth of myometrial invasion were available for 40 EC patients. Pie charts show the distribution of smaller (≤2 cm) and bigger (>2 cm) tumors (FIG. 8A), as well as the proportion of presence of the myometrial invasion (FIG. 8B) among low-grade EEC and other EC. There was no significant difference (ns) in the distribution of these tumor characteristics between EC histological types (calculated by Fisher's exact test). Scatter dot plots show the tumor size (measured in cm) (FIG. 8C) and the depth of myometrial invasion (measured in mm) (FIG. 8D) for the low-grade EEC and other EMC. A horizontal line indicates a mean, and asterisks indicate P values (* P<0.05). There was no significant (ns) difference between mean tumor sizes among the EC histological types. However, within tumors with myometrial invasion (n=40), other EMC subtypes had deeper myometrial invasion compared to low-grade EEC. The significant differences were assessed using an unpaired two-tailed t-test.

FIGS. 9A, 9B, 9C, and 9D show cervicovaginal levels of proteins in all endometrial cancer patients stratified based on the tumor characteristics. A volcano plot analysis was used to assess differences in the protein levels among patients with all endometrial cancer stratified based on tumor characteristics, such as tumor size (≤2 cm vs. >2 cm) (FIG. 9A), histological type (low grade endometrial endometrioid carcinoma (EEC) vs. other endometrial cancer (EC) types) (FIG. 9B), presence of myometrial invasion (no vs. yes) (FIG. 9C), and mismatch repair (MMR) protein status (MMR-proficient vs. MMR-deficient) (FIG. 9D). Statistical significance was determined using multiple t-tests with the false discovery rate (FDR) correction. Proteins with q<0.01 were considered significant. Scatter dot plots show concentrations of identified protein biomarkers in individual samples. A horizontal line indicates the mean and asterisks indicate P values (* P<0.05; ** P<0.01; *** P<0.001; **** P<0.0001).

FIG. 10 shows the study workflow. The study workflow is summarized here. The full inclusion and exclusion criteria are included. Briefly, we enrolled a total of 192 women. Based on histopathological results, 108 patients had benign conditions, 18 patients had endometrial hyperplasia and 66 had EC. The EC group was further stratified into grade 1 or 2 endometrial endometrioid carcinoma (EEC) (n=53) and other EC (n=13), which included grade 3 EEC and other non-endometrioid histopathological subtypes.

FIGS. 11A, 11B, and 11C shows unsupervised hierarchical clustering reveals two distinct patient clusters based on their cervicovaginal protein levels. FIG. 11A shows a heatmap of the relative levels of proteins across all samples. Prior to analysis, data were mean-centered, and variance scaled along each row. Euclidean distance and Ward linkage method were used for clustering. The analysis revealed two distinct patient clusters. FIG. 11B shows pie charts illustrate the patient demographics of the two clusters, based on disease group, menopausal status, and BMI group. Disease group and menopausal status were statistically different among the clusters. Subcluster analysis of cluster 2 illustrated disease and BMI group were statistically different between cluster 2A and 2B. FIG. 11C shows statistical difference was assessed using the chi-square test. Asterisks indicate the p value (* p<0.05, **** p<0.0001, ns (not significant)).

FIGS. 12A and 12B show numerous growth and angiogenic markers are significantly upregulated in patients with EC. FIG. 12A shows a heatmap demonstrates proteins that are statistically up-or downregulated (fold change (FC)) in endometrial cancer (n=66), grade 1/2 EEC (n=53), or other EC types (n=13) compared to benign patients. Statistical difference was assessed using a two-sample t-test with FDR correction. Asterisks indicate the q value (* q<0.05, ** q<0.01, *** q<0.001, **** q<0.0001). FIG. 12B shows truncated violin plots depict the concentration of key growth factors that are statistically elevated in grade 1/2 EEC and other EC types compared to benign patients. P values were calculated using a 1-way ANOVA with Bonferroni's correction. The horizontal line represents the median value, and the asterisks indicate the p value (* p<0.05, ** p<0.01, *** p<0.001, **** p<0.0001).

FIGS. 13A, 13B, 13C, and 13D shows growth factors in cervicovaginal lavage show good to excellent discriminatory potential for endometrial cancer. FIG. 13A shows univariate ROC analysis assessed the potential of proteins for discriminating EC all, grade 1/2 EEC, and other EC types from benign patients. Angiopoietin-2 and VEGF-A have good discriminatory potential for all endometrial cancers (FIG. 13B), grade 1/2 EEC (FIG. 13C), and angiopoietin-2 (FIG. 13D) has excellent discriminatory potential for other EC types. In addition, endoglin, FAP, MIA, and VEGF-A have good discriminatory potential for other EC types. The discriminatory potential was based on the area under the curve (AUC) values. Proteins with an AUC of above or equal to 0.8 were considered good discriminators, and proteins with an AUC of above or equal to 0.9 were considered excellent discriminators.

FIGS. 14A, 14B, and 14C shows ROC analysis demonstrates the diagnostic utility of a multivariate protein model with metadata for EC. FIG. 14A shows multivariate ROC curve analysis resulted in an excellent AUC of 0.918. To build the model, 11 proteins were selected based on high least absolute shrinkage and selection operator (LASSO) frequencies and empirical testing. Age and BMI were added to the model as they are known risk factors for EC. CA125, CA19-9, IL-10, MCP-1, TIM-3, TGF-α, TNF-α, and VEGF-A data were evaluated Example 1. FIG. 14B shows a scatter plot illustrates the Monte Carlo cross-validation (MCCV) with a predictive accuracy of 86%. The predicted class probabilities of the samples are shown, using the classifier at a threshold of 0.5. FIG. 14C shows the confusion matrix depicts the number of times this combination of proteins accurately classified patients to the disease groups. This multivariate model accurately classified 156/174 samples with a specificity of 90.7% and a sensitivity of 87.8%. FIGS. 15A, 15B, 15C, and 15D shows key growth factors in cervicovaginal lavages aligned with tumor characteristics demonstrating prognostic utility. Truncated violin plots depict several proteins are elevated in patients with more advanced tumor grade (FIG. 15A), larger tumor size (FIG. 15B), in tumors with a presence of myometrial invasion (FIG. 15C), and MMR deficiency (FIG. 15D). P values were calculated using Student's t-test or a 1-way ANOVA with Bonferroni's correction and asterisks indicate the p value (* p<0.05, ** p<0.01, *** p<0.001, **** p<0.0001).

FIG. 16 shows protein concentrations in varying severities of EC compared to benign conditions. Truncated violin plots depict the concentrations of 14 proteins across all the disease groups (Benign, hyperplasia, grade 1/2 EEC, and other EC types). The solid horizontal line represents the median value. P values were calculated using a one-way ANOVA with Bonferroni's correction. Asterisks indicates the p values (* p<0.05, ** p<0.01, *** p<0.001).

FIG. 17 shows a univariates ROC curve of proteins discriminatory potential for EC types. A univariate receiver operating characteristics (ROC) analysis was used to assess the potential for individual proteins to discriminate EC types (EC all, Grade 1/2 EEC, and Other EC types) from benign conditions. The area under the curve (AUC) was calculated for each protein. Proteins with an AUC above 0.8 were considered good discriminatory biomarkers. Three proteins (endoglin, FAP, and MIA) displayed good discriminatory potential for other EC types.

FIGS. 18A, 18B and 18C shows a multivariate protein model of 11 proteins shows excellent discriminatory potential for EC. FIG. 18A shows multivariate ROC curve of 11 proteins displays excellent discriminatory potential for EC with an area under the curve (AUC) of 0.923. Proteins were selected based on high least absolute shrinkage and selection operator (LASSO) frequencies and empirical test of combinations of proteins. A Monte Carlo cross validation (MCVV) (FIG. 18B) was used to test the predictive accuracy of the model, which was found to be 83.7%. The predicted class probabilities of the sample are shown using the classifier at a threshold of 0.5. FIG. 18C shows the confusion matrix depicting the number of patients the protein model accurately classified to their respective disease groups. This multivariate model accurately classified 151/174 samples with a specificity of 87% and a sensitivity of 86.4%.

FIG. 19 shows protein concentrations positively correlated with tumor size and depth of myometrial invasion. Spearman rank correlation coefficients were calculated between protein concentrations and depth of myometrial invasion (measured in mm) and tumor size (measured in cm). These values are represented in a heatmap. P values are represented with black circles * p<0.05, ** p<0.01, *** p<0.001, **** p<0.0001.

FIG. 20 shows correlation of pro-and anti-inflammatory cytokines, chemokines, and growth factors. Heatmap depicts correlation coefficients of 90 proteins with one another. Spearman rank correlation analysis was utilized to calculate correlation coefficients. Hierarchical cluster analysis was conducted, data was not centered or scaled. Euclidean distance and Ward linkage methods were used for clustering.

FIGS. 21A and 21B shows Angiopoietin-2, FAP, and VEGF-A positively correlate with each other to promote angiogenesis. Spearman rank correlation analysis revealed that Angiopoietin-2 positively correlates with VEGF-A and FAP, also VEGF-A positively correlates with FAP in the EC patients (FIG. 21A). Spearman rank correlation analysis was also conducted on the levels of angiopoietin-2, FAP, and VEGF-A in benign patients (FIG. 21B).

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

The term “cancer” refers to any physiological condition in mammals characterized by unregulated cell growth. Cancers described herein include solid tumors. A “solid tumor” or “tumor” refers to a lesion and neoplastic cell growth and proliferation, whether malignant or benign and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The term “hyperplasia” may refer to when healthy cells undergo abnormal changes within tissues or organs, and it is considered a pre-cancerous disease state. In some embodiments, hyperplasia may progress and become cancer. In other embodiments, hyperplasia may regress. The term “pre-cancerous disease state” may refer to a condition or lesion involving abnormal cells associated with an increased risk of developing into cancer. In some embodiments, the progression of normal cells to precancerous cells and towards endometrial cancer may involve oncogenes, inflammation, and multiple somatic mutations that initiate the malignant transformation, activation, and clonal expansion of stem cells.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder, or condition described herein. In another embodiment, the subject is a mammal (e.g., a human) at risk of developing a disease, disorder, or condition described herein. In certain instances, the term patient refers to a human.

As used herein, the terms “normal subject,” “healthy subject,” or “control subject,” or “control,” may be used interchangeably and refers to a subject with benign conditions. In some embodiments, a healthy subject may refer to a subject undergoing a hysterectomy for a benign condition, e.g., abnormal uterine bleeding, endometriosis, pelvic pain, etc.

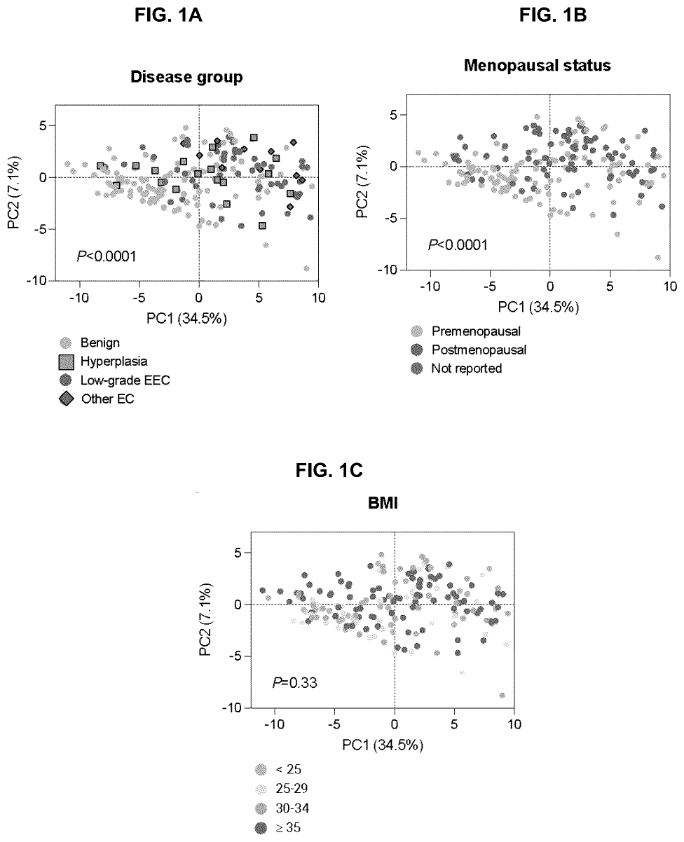

Referring now to FIGS. 1A-21B, the present invention features methods (e.g., non/minimally invasive methods) for improving early endometrial cancer (EC; e.g., cancer within the upper reproductive tract) detection/diagnosis among diverse racial and ethnic populations by developing cost-effective, robust non-invasive diagnostics that facilitate a better understanding and decrease morbidity associated with this cancer health disparity in women.

The present invention features a method (e.g., a non/minimally invasive method) comprising obtaining a cervicovaginal lavage (CVL) sample from a patient, producing a profile of the CVL sample collected in the preceding step by detecting at least five or more protein biomarkers and analyzing the CVL sample profile produced in the prior step. In some embodiments, the protein biomarkers are cervicovaginal protein biomarkers. In other embodiments, the method comprises obtaining a cervicovaginal lavage (CVL) sample from a patient, producing a profile of the CVL sample collected in the preceding step by detecting at least five or more protein biomarker and analyzing the CVL sample profile produced in the prior step. In some embodiments, the protein biomarkers are cervicovaginal protein biomarkers.

In other embodiments, the present invention features a method comprising a) obtaining a biological sample (e.g., CVL sample) from a patient, b) producing a profile of the biological sample (e.g., CVL sample) collected in (a) by detecting at least five protein biomarkers selected from the group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3), and c) analyzing the biological sample (e.g., CVL sample) profile produced in (b). In some embodiments, the method comprises producing a profile of the biological sample (e.g., CVL sample) collected in (a) by detecting at least ten protein biomarkers.

In some embodiments, the aforementioned methods comprise detecting at least three or more protein biomarkers. In some embodiments, the method comprises detecting at least four or more protein biomarkers. In some embodiments, the method comprises detecting at least five or more protein biomarkers. In some embodiments, the method comprises detecting at least six or more protein biomarkers. In some embodiments, the method comprises detecting at least seven or more protein biomarkers. In some embodiments, the method comprises detecting at least eight or more protein biomarkers. In some embodiments, the method comprises detecting at least nine or more protein biomarkers. In some embodiments, the method comprises detecting at least ten or more protein biomarkers. In some embodiments, the method comprises detecting at least eleven or more protein biomarkers. In some embodiments, the method comprises detecting at least twelve or more protein biomarkers. In some embodiments, the method comprises detecting at least thirteen or more protein biomarkers. In some embodiments, the method comprises detecting at least fourteen or more protein biomarkers. In some embodiments, the method comprises detecting at least fifteen or more protein biomarkers.

In certain embodiments, the method comprises detecting eleven protein biomarkers. For example, the protein biomarkers may include angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3). In some embodiments, angiopoietin-2, endoglin, FAP, ferritin, FGF-1, MIA, HB-EGF, and VEGF-A are upregulated, whereas galectin-3, MPO, and IGFBP3 are downregulated in all endometrial cancer when compared to benign patients.

In some embodiments, the protein biomarkers may further comprise cytokine, growth factors, immune checkpoint markers, apoptosis markers, and tumor markers. In some embodiments, the cytokines comprise IL-10, MCP-1, MDC, and TNFα. In some embodiments, the growth factors comprise TGF-α and VEGF. In some embodiments, the immune checkpoint markers comprise TIM-3. In some embodiments, the apoptosis markers comprise TRAIL. In some embodiments, the tumor markers comprise CYFRA 21-1. In some embodiments, the protein biomarkers (i.e., cervicovaginal protein biomarkers) are selected from a group consisting of TIM-3, IL-10, TRAIL, TGF-α, CYFRA 21-1, VEGF-A, and TNFα.

In other embodiments, the protein biomarkers (i.e., cervicovaginal protein biomarkers) are selected from a group consisting of angiopoietin-2, endoglin, FAP, ferritin, FGF-1, MIA, HB-EGF, and VEGF-A are upregulated, whereas galectin-3, MPO, IGFBP3, TIM-3, IL-10, TRAIL, TGF-α, CYFRA 21-1, VEGF-A, TNFα, IL-6, SCF, fractalkine, IP-10, MCP-1, MCP-3, MIP-1α, MIP-1β, PDGF-AA, leptin, AFP, CA15-3, CD40, CA125, CA19-9, MDC, and PD-L2.

In some embodiments, the protein biomarkers (i.e., cervicovaginal protein biomarkers) may be upregulated in EC, e.g., angiopoietin-2, endoglin, FAP, ferritin, FGF-1, MIA, HB-EGF, and VEGF-A. In other embodiments, the protein biomarkers (i.e., cervicovaginal protein biomarkers) may be downregulated in EC, e.g., galectin-3, MPO, and IGFBP3.

In some embodiments, the biological sample comprises a cervicovaginal lavage (CVL) sample, a urine sample, a vaginal swab or vaginal fluid, or cervicovaginal secretion. The cervicovaginal secretion may be collected via a physician, self-collected lavage, or a menstrual cup. In some embodiments, the cervicovaginal lavage (CVL) is obtained by a physician. In other embodiments, the cervicovaginal lavage (CVL) is obtained by the subjects themselves.

Various methods may be used to produce a profile in accordance with the present invention. In some embodiments, a bioinformatic pipeline may be used to build and predict said profile.

In some embodiments, analyzing the biological sample (e.g., CVL sample) profile comprises comparing the biological sample (e.g., CVL sample) profile to a healthy control profile. In other embodiments, analyzing the biological sample (e.g., CVL sample) profile comprises comparing a baseline profile to a second profile.

In some embodiments, the healthy control profile is obtained from a healthy control subject. For example, a biological sample (e.g., CVL sample) may be obtained from the healthy control subject, and a profile of the CVL sample may be produced by detecting at least five or more protein biomarkers.

In some embodiments, a profile of a biological sample (e.g., CVL sample) is produced by detecting at least three or more protein biomarkers. In some embodiments, a profile of a biological sample (e.g., CVL sample) is produced by detecting at least four or more protein biomarkers. In some embodiments, a profile of a biological sample (e.g., CVL sample) is produced by detecting at least five or more protein biomarkers. In some embodiments, a profile of a biological sample (e.g., CVL sample) is produced by detecting at least six or more protein biomarkers. In some embodiments, a profile of a biological sample (e.g., CVL sample) is produced by detecting at least seven or more protein biomarkers. In some embodiments, a profile of a biological sample (e.g., CVL sample) is produced by detecting at least eight or more protein biomarkers. In some embodiments, a profile of a biological sample (e.g., CVL sample) is produced by detecting at least nine or more protein biomarkers. In some embodiments, a profile of a biological sample (e.g., CVL sample) is produced by detecting at least ten or more protein biomarkers. In some embodiments, a profile of a biological sample (e.g., CVL sample) is produced by detecting at least eleven or more protein biomarkers. In some embodiments, a profile of a biological sample (e.g., CVL sample) is produced by detecting at least twelve or more protein biomarkers. In some embodiments, a profile of a biological sample (e.g., CVL sample) is produced by detecting at least thirteen or more protein biomarkers. In some embodiments, a profile of a biological sample (e.g., CVL sample) is produced by detecting at least fourteen or more protein biomarkers. In some embodiments, a profile of a biological sample (e.g., CVL sample) is produced by detecting at least fifteen or more protein biomarkers. In some embodiments, a profile of a biological sample (e.g., CVL sample) is produced by detecting at least twenty or more protein biomarkers.

The methods described herein may predict the risk of or diagnose endometrial cancer, e.g., EC type 1, in women. In other embodiments, the methods described herein may determine the prognosis of endometrial cancer, e.g., EC type 1, in women.

The present invention may feature a method of diagnosing endometrial cancer (EC) in a subject in need thereof. The method may comprise a) obtaining a biological sample (e.g., CVL sample) from the subject, b) producing a profile of the biological sample (e.g., CVL sample) collected by detecting at least five or more protein biomarkers, and c) analyzing the biological sample (e.g., CVL sample) profile produced. The subject is diagnosed with EC if the levels of at least five biomarkers are altered compared to a healthy control profile. In some embodiments, the subject is diagnosed with EC if the levels of at least five biomarkers are elevated compared to a healthy control profile.

In some embodiments, the subject is diagnosed with EC if the levels of at least ten biomarkers are altered, e.g., elevated, compared to a healthy control profile. In some embodiments, the subject is diagnosed with EC if the levels of at least fifteen biomarkers are altered, e.g., elevated, compared to a healthy control profile. In some embodiments, the subject is diagnosed with EC if the levels of at least twenty biomarkers are altered, e.g., elevated, compared to a healthy control profile.

In other embodiments, the method of diagnosing endometrial cancer (EC) in a subject in need thereof comprising: a) obtaining a biological sample (e.g., CVL sample) from the subject, b) producing a profile of the biological sample collected in (a) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3), and c) analyzing the biological sample (e.g., CVL sample) profile produced in (b). In some embodiments, the method comprises producing a profile of the biological sample (e.g., CVL sample) collected in (a) by detecting at least ten protein biomarkers. In some embodiments, the subject is diagnosed with EC if the levels of at least five or ten biomarkers are altered compared to a healthy control profile. For example, the subject may be diagnosed with EC if the levels of angiopoietin-2, endoglin, FAP, ferritin, FGF-1, MIA, HB-EGF, or VEGF-A are elevated compared to the healthy control and the levels of galectin-3, MPO, or IGFBP-3 are decrease compared to the healthy control profile. The present invention may also feature methods of treating endometrial cancer (EC) in a subject in need thereof, where if a subject is diagnosed with EC, then an EC treatment is administered to the subject. In addition, in some embodiments, the present invention may feature methods of monitoring a treatment administered to the subject.

In some embodiments, the protein biomarkers are cervicovaginal protein biomarkers and may be selected from a group comprising angiopoietin-2, endoglin, FAP, ferritin, FGF-1, MIA, HB-EGF, and VEGF-A are upregulated, whereas galectin-3, MPO, IGFBP3, TIM-3, IL-10, TRAIL, TGF-α, CYFRA 21-1, VEGF, TNFα, IL-6, SCF, fractalkine, IP-10, MCP-1, MCP-3, MIP-1α, MIP-1β, PDGF-AA, leptin, AFP, CA15-3, CD40, CA125, CA19-9, MDC, PD-L2, or a combination thereof.

The present invention features a method of treating endometrial cancer (EC) in a subject in need thereof. The method comprises diagnosing the subject with EC. In some embodiments, the subject is diagnosed with EC by obtaining a biological sample (e.g., CVL sample) from a patient, producing a profile of the CVL sample collected in the preceding step by detecting at least five or more protein biomarkers and analyzing the biological sample (e.g., CVL sample) profile produced in the prior step. In some embodiments, if the levels of at least five biomarkers are altered, the subject is diagnosed with EC. The method may further comprise administering a treatment to the subject. In some embodiments, the biomarkers are elevated compared to a healthy control profile. In other embodiments, the biomarkers are reduced compared to a healthy control profile.

In some embodiments, the method of treating endometrial cancer (EC) in a subject in need thereof comprises: a) diagnosing the subject with EC by: i) obtaining a biological sample from the subject, ii) producing a profile of the biological sample collected in (i) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3); and iii) analyzing the biological sample profile produced in (ii) and b) administering an EC treatment to the subject and monitoring the therapy. In some embodiments, the subject is diagnosed with EC if the levels of at least five biomarkers are altered compared to a healthy control profile. In some embodiments, the method comprising producing a profile of the biological sample (e.g., CVL sample) collected in (a) by detecting at least ten protein biomarkers. In some embodiments, the subject is diagnosed with EC if the levels of at least ten biomarkers are altered compared to a healthy control profile. For example, the subject may be diagnosed with EC if the levels of angiopoietin-2, endoglin, FAP, ferritin, FGF-1, MIA, HB-EGF, or VEGF-A are elevated compared to the healthy control and the levels of galectin-3, MPO, or IGFBP-3 are decrease compared to the healthy control profile.

Without wishing to limit the present invention to any theory or mechanism, it is believed that different patients will exhibit different biomarker patterns, e.g., biomarker profiles; thus, machine learning algorithms will be used to compare the profile from a patient to a profile from a healthy control subject.

In some embodiments, the subject is diagnosed with EC if the levels of at least five biomarkers are elevated compared to a healthy control profile. In some embodiments, the subject is diagnosed with EC if the levels of at least six biomarkers are elevated compared to a healthy control profile. In some embodiments, the subject is diagnosed with EC if the levels of at least seven biomarkers are elevated compared to a healthy control profile. In some embodiments, the subject is diagnosed with EC if the levels of at least eight biomarkers are elevated compared to a healthy control profile. In some embodiments, the subject is diagnosed with EC if the levels of at least nine biomarkers are elevated compared to a healthy control profile. In some embodiments, the subject is diagnosed with EC if the levels of at least ten biomarkers are elevated compared to a healthy control profile. In some embodiments, the subject is diagnosed with EC if the levels of at least eleven biomarkers are elevated compared to a healthy control profile. In some embodiments, the subject is diagnosed with EC if the levels of at least twelve biomarkers are elevated compared to a healthy control profile. In some embodiments, the subject is diagnosed with EC if the levels of at least thirteen biomarkers are elevated compared to a healthy control profile. In some embodiments, the subject is diagnosed with EC if the levels of at least fourteen biomarkers are elevated compared to a healthy control profile. In some embodiments, the subject is diagnosed with EC if the levels of at least fifteen biomarkers are elevated compared to a healthy control profile. In some embodiments, the subject is diagnosed with EC if the levels of at least twenty biomarkers are elevated compared to a healthy control profile.

In some embodiments, the profiles are analyzed by using machine learning models (e.g., random forest or logistic regression).

In some embodiments, other symptoms may be used to diagnose EC in a subject.

The present may also feature a method of monitoring a treatment for endometrial cancer (EC) in a subject in need thereof. The method comprises obtaining a first cervicovaginal lavage (CVL) sample from the subject and producing a baseline profile of the CVL sample collected by detecting at least two or more protein biomarkers. In some embodiments, the method comprises administering the treatment for EC to the subject. The method may further comprise obtaining a second cervicovaginal lavage (CVL) sample from the subject and producing a second profile of the CVL sample collected by detecting at least two or more protein biomarkers. In some embodiments, the method comprises comparing the baseline profile of the CVL sample to the second profile of the CVL sample. In some embodiments, the treatment is effective if the levels of at least two biomarkers are altered from the baseline profile as compared to the second profile, e.g., the treatment is effective if the levels of at least two biomarkers are decreased from the baseline profile as compared to the second profile.

In some embodiments, the treatment is effective if the levels of at least two biomarkers from the baseline profile are decreased as compared to the second profile. In some embodiments, the treatment is effective if the levels of at least one biomarker from the baseline profile are decreased as compared to the second profile. In some embodiments, the treatment is effective if the levels of at least five biomarkers from the baseline profile are decreased as compared to the second profile. In some embodiments, the treatment is effective if the levels of at least ten biomarkers from the baseline profile are decreased as compared to the second profile. In some embodiments, the treatment is effective if the levels of at least twenty biomarkers from the baseline profile are decreased as compared to the second profile.

In some embodiments, producing the baseline profile comprises detecting at least five or more biomarkers. In other embodiments, producing the baseline profile comprises detecting at least five or more biomarkers. In some embodiments, producing the second profile comprises detecting at least five or more biomarkers. In other embodiments, producing the second profile comprises detecting at least five or more biomarkers.

In other embodiments, the method of monitoring a treatment for endometrial cancer (EC) in a subject in need thereof comprises a) obtaining a first biological sample from the subject; b) producing a baseline profile of the first biological sample collected in (a) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3); c) administering the treatment for EC to the subject; d) obtaining a second biological sample from the subject; e) producing a second profile of the second biological sample collected in (d) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3) and f) comparing the baseline profile of the first biological sample produced in (b) to the second profile of the second biological sample produced in (e); wherein the treatment is effective if the levels of at least five biomarkers are altered from the baseline profile as compared to the second profile. In some embodiments, the treatment is effective if the levels of at least ten biomarkers are altered from the baseline profile as compared to the second profile. In some embodiments, the method comprises producing a profile of the biological sample (e.g., CVL sample) collected in (a) by detecting at least ten protein biomarkers.

In some embodiments, the treatment is effective if the levels of angiopoietin-2, endoglin, FAP, ferritin, FGF-1, MIA, HB-EGF, or VEGF-A are reduced from the baseline profile as compared to the second profile and/or the levels of galectin-3, MPO, or IGFBP-3 are elevated from the baseline profile as compared to the second profile. In other embodiments, if the levels of angiopoietin-2, endoglin, FAP, ferritin, FGF-1, MIA, HB-EGF, or VEGF-A are elevated from the baseline profile as compared to the second profile and/or the levels of galectin-3, MPO, or IGFBP-3 are reduced from the baseline profile as compared to the second profile, the treatment administered to the subject may be changed.

In some embodiments, the methods described herein are in vitro and are not carried out directly on the subject.

The present invention may also feature an in vitro method of diagnosing endometrial cancer (EC) in a subject in need thereof. The method may comprise producing a profile from a cervicovaginal lavage (CVL) sample obtained from a subject by detecting at least two or more protein biomarkers and analyzing the CVL sample profile produced. In some embodiments, the subject is diagnosed with EC if the levels of at least two biomarkers are altered compared to a healthy control profile. The present invention may also feature methods of treating endometrial cancer (EC) in a subject in need thereof, where if a subject is diagnosed with EC, then an EC treatment is administered to the subject.

The present invention may further feature an in vitro method comprising producing a profile from a cervicovaginal lavage (CVL) sample obtained from a subject by detecting at least two or more protein biomarkers and analyzing the CVL sample profile produced. In some embodiments, the method predicts the risk of endometrial cancer, e.g., EC type 1, in women. In other embodiments, the method diagnoses endometrial cancer, e.g., EC type 1, in women.

In some embodiments, the present invention features method of determining a prognosis of endometrial cancer (EC) in a subject in need thereof, the method comprising: a) obtaining a biological sample from the subject, b) producing a profile of the biological sample collected in (a) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3), and c) analyzing the biological sample profile produced in (b); wherein the subject has a poor prognosis if the levels of angiopoietin-2, endoglin, FAP, ferritin, FGF-1, MIA, HB-EGF, or VEGF-A are elevated compared to the healthy control and/or the levels of galectin-3, MPO, or IGFBP-3 are decrease compared to the healthy control profile.

In some embodiments, methods described herein may further comprise characterizing endometrial tumor characteristics, including tumor size, myometrial invasion, mismatch repair (MMR) status, histological grade, or a combination thereof. In some embodiments, generating a profile may further include a subject's age and characterize endometrial tumor features, such as tumor size, depth of myometrial invasion, mismatch repair (MMR) status, histological grade, or any combination thereof.

### Example 1

The following is a non-limiting example of the present invention. It is to be understood that said example is not intended to limit the present invention in any way. Equivalents or substitutes are within the scope of the present invention.

Study participants. Participants were recruited at three clinical sites located in the Phoenix (AZ, USA) metropolitan area between June 2018 and February 2020. One hundred ninety-two women undergoing hysterectomy for benign or malignant indications were enrolled and contributed to the study. Classification of women to four disease groups: benign conditions (n=108), endometrial hyperplasia (n=18), low-grade (grade 1 or 2) endometrioid carcinoma (EEC) (n=53), and other EC (including grade 3 EEC or other histological subtypes) (n=13) was based on histopathology of biopsy samples collected after the surgery. Women of any race or ethnicity and aged 18 years or older were included. Exclusion criteria included: currently menstruating; currently lactating; currently on antibiotics, antifungals, antivirals, or topical steroids; current vaginal infection (bacterial vaginosis, candidiasis), vulvar infection, urinary tract infection, or sexually transmitted infection (chlamydia, gonorrhea, trichomoniasis, genital herpes) or within the previous three weeks; use of douching substances, vaginal medications, vaginal suppositories, feminine deodorant sprays, wipes, or lubricants within the previous 48 hours; use of depilatory treatments in the genital area within the previous 72 hours; any skin condition in the genital area interfering with the study; sexual intercourse within the previous 48 hours; bath or swimming within the previous 4 hours; smoking or consuming nicotine-contained products within the previous 2 hours; hepatitis; being HIV-positive. The exclusion criteria were verified by a physician's pelvic exam, medical record and/or self-reported. Demographic, socioeconomic, and medical history data were collected from surveys and/or medical records.

Sample collection and processing. Clinical specimens were collected by a surgeon in the operating room during the standard-of-care hysterectomy procedure. All samples were obtained after anesthesia and prior to vaginal sterilization. Cervicovaginal lavage (CVL) samples were collected using a non-lubricated speculum and 10 ml of sterile 0.9% saline solution (Teknova, Hollister, CA). Following the collection, CVL samples were immediately placed on ice and frozen at −80° C. within 1 hour. Prior to downstream analyses, CVL samples were thawed on ice, clarified by centrifugation (700×g for 10 min at 4° C.) and aliquoted to avoid multiple freeze-thaw cycles. All samples were stored at −80° C.

Quantification of soluble proteins. Levels of 71 proteins (AFP, BTLA, CA15-3, CA19-9, CA125, CD27, CD28, CD40, CD80, CD86, CEA, CYFRA21-1, EGF, eotaxin/CCL11, Flt-3L, FGF-2, fractalkine/CX3CL1, G-CSF, GITRL, GROα/CXCL1, GM-CSF, HE4, HGF, HVEM, ICOS, IFNα2, IFNγ, IL-1α, IL-1β, IL-2, IL-4, IL-5, IL-6, IL-7, IL-8/CXCL8, IL-9, IL-10, IL-12 (p40), IL-12 (p70), IL-13, IL-15, IL-17A, IP-10/CXCL10, LAG-3, leptin, MCP-1/CCL2, MCP-3/CCL7, MDC/CCL22, MIF, MIP-1α/CCL3, MIP-1β/CCL4, OPN, PD-1, PD-L1, PD-L2, PDGF-AA, PDGF-AB/BB, prolactin, PSA (total), RANTES/CCL5, SCF, sCD40L, sFas, sFasL, TGF-α, TIM-3, TLR2, TNFα, TNFβ, TRAIL, VEGF) were measured in CVL samples using the Milliplex MAP Magnetic Bead Immunoassays: Human Cytokine Chemokine Panel 1, Human Circulating Cancer Biomarker Panel 1 and Human Immuno-Oncology Checkpoint Protein Panel 1 (Millipore, Billerica, MA) in accordance with the manufacturer's protocols. Data were collected with a Bio-Plex 200 instrument and analyzed using Manager 5.0 software (Bio-Rad, Hercules, CA). Levels of IL-36γ (IL-1F9) were measured in CVL samples by enzyme-linked immunosorbent assay using Human IL-36γ ELISA kit (RayBiotech, Norcross, GA) in accordance with the manufacturer's instruction. A five-parameter logistic regression curve fit was used to determine the concentration. All samples were assayed in duplicate. The concentration values below the detection limit were substituted with 0.5 of the minimum detectable concentration provided in the manufacturer's instructions. The logarithmic transformation was applied to normalize the data.

Unsupervised data reduction analyses. The principal component analysis was performed to reduce the observed variables into a smaller number of principal components that account for most of the variance in the observed variables. For the first two principal components (PC1 and PC2), the difference among groups was assessed by the multivariate analysis of variance model. The statistical differences for individual components were assessed using an analysis of variance. If the overall difference was significant (P<0.05), pairwise comparisons with Bonferroni adjustment were performed. The hierarchical clustering analysis was performed to show relationships of protein biomarker levels to metadata available for each patient, i.e., disease group, menopausal status, and BMI. Prior to clustering, levels of each protein biomarker were mean centered and then variance was scaled. Hierarchical clustering was performed using ClustVis server and based on Euclidean distance and Ward linkage. The statistical differences in distribution of patient-related factors between clusters were assessed using Fisher's exact test or chi square test.

Receiver operating characteristics (ROC) analysis. The univariate ROC analysis was performed to identify protein biomarkers that discriminate against specific disease groups with high sensitivity and specificity. The mean levels of proteins for each patient were used in the analyses. The strength of the discriminators was measured with area under the curve (AUC) values. Proteins with AUC greater than or equal to 0.8, or 0.9 were considered as good, or excellent discriminators, respectively.

Supervised machine-learning analyses. Supervised learning was performed using the logistic regression algorithm. The features were selected based on the least absolute shrinkage and selection operator (LASSO) modeling. The performance of the predictive model was evaluated using the Monte Carlo cross-validation, which uses ⅔ of samples for model training and the remaining ⅓ of samples for testing. One hundred cross validations were performed, and the results were averaged to generate plots. Evaluation metrics included the AUC of multivariate ROC analysis and the confusion matrix calculated at a probability threshold of 0.5. The analysis was performed using MetaboAnalyst 5.0.

Volcano plot and correlation analyses. Differences in the protein biomarker levels among patients diagnosed with EC stratified based on histological type and grade, tumor size, presence of myometrial invasion, and MMR status were tested using multiple t-tests and corrected using false discovery rate (FDR) method. Differences in mean protein levels and q values were graphically presented as volcano plots. Protein biomarkers with q<0.1 were considered significant. The Spearman's rank correlation analysis was also performed to investigate the association of protein biomarker levels with the tumor size (measured in cm) and the depth of myometrial invasion (measured in mm). A correlation matrix was computed using correlation coefficients (r) with P values, and graphically presented as a heat map. P<0.05 was considered significant.

Other statistical analyses. Differences in the demographic, socioeconomic and other patient-related variables between disease groups were tested using the Kruskal-Wallis test for continuous variables and Fisher's exact test for categorical variables. The statistical differences in the concentrations of protein biomarkers among the patient groups were tested using a linear mixed effects model where the group was a fixed effect and the replicate was the random effect. If the overall difference was significant (P<0.05), paired tests were performed with Bonferroni adjustment. Comparisons were adjusted for age and BMI in the linear mixed effects models by including these variables as predictors in the models, in addition to indicators for the patient groups (with benign as the reference group). Statistical analyses were performed using SAS 9.4 (SAS Institute, Cary, NC) unless otherwise indicated.

Study population. A total of 192 women undergoing hysterectomy were recruited and enrolled in this cross-sectional study (Table 1). Women were classified into four disease groups: benign conditions (n=108), endometrial hyperplasia (n=18), low-grade endometrioid carcinoma (EEC) (n=53), and other EC subtypes (n=13). The classification into groups was based on the histology of biopsy samples. The average age and body mass index (BMI) were 51 years and 34.8 kg/m², respectively. Regarding race and ethnicity, participants were predominantly Caucasian (74.7%), with a relatively high proportion of women identifying as Hispanic (26.2%). Women diagnosed with low-grade EEC and other EC subtypes were older (mean 58.7 and 60.8 years, respectively) compared to women diagnosed with benign conditions 45.6 years; P<0.0001) and mostly postmenopausal (76.5% and 92.3% vs. 17.6%; P<0.0001). Women with low-grade EEC also had higher body mass index (BMI; mean 40.3 kg/m²) than women with benign conditions (mean 30.6 kg/m²; P<0.0001). In addition, there were significant differences in other comorbidities, such as diabetes (P=0.006) and hypertension (P=0.002) among the groups; however, these differences were attenuated after controlling for BMI or age.

TABLE 1
Patient demographics. Race and menopause status data were available for 190 women;
ethnicity data were available for 191 women. P values were calculated using Kruskal-
Wallis test for continuous variables and Fisher's exact test for categorical variables.

| | | | | | Other | |
|---|---|---|---|---|---|---|
| | | | | Low-grade | endometrial | |
| | | Benign | Endometrial | endometrioid | cancer | |
| | All | conditions | hyperplasia | carcinoma | subtypes | P value |
| | (n = 192) | (n = 108) | (n = 18) | (n = 53) | (n = 13) | overall |
| Age [mean (SD)] | 51.02 (12.45) | 45.55 (10.01) | 54.11 (13.35) | 58.73 (11.82) | 60.77 (8.06) | <0.0001 |
| (n = 192) | | | | | | |
| Race [n (%)] | | | | | | |
| (n = 190) | | | | | | |
| White/Caucasian | 142 (74.74) | 78 (72.90) | 16 (88.89) | 37 (71.15) | 11 (84.62) | 0.16 |
| American Indian/ | 15 (7.89) | 5 (4.67) | 1 (5.56) | 8 (15.38) | 1 (7.69) | |
| Alaska Native | | | | | | |
| Black/African | 12 (6.32) | 11 (10.28) | 0 (0.00) | 1 (1.92) | 0 (0.00) | |
| American | | | | | | |
| Other | 21 (11.05) | 13 (12.15) | 1 (5.56) | 6 (11.54) | 1 (7.69) | |
| Ethnicity [n (%)] | | | | | | |
| (n = 191) | | | | | | |
| Hispanic | 50 (26.18) | 32 (29.63) | 4 (22.22) | 11 (21.15) | 3 (23.08) | 0.67 |
| Non-Hispanic | 141 (73.82) | 76 (70.37) | 14 (77.78) | 41 (78.85) | 10 (76.92) | |
| Body Mass Index | | | | | | |
| [n (%)] (n = 192) | | | | | | |
| <25 | 29 (15.38) | 23 (21.30) | 0 (0.00) | 4 (7.55) | 2 (15.38) | <0.0001 |
| 25-29 | 47 (24.38) | 38 (35.19) | 1 (5.56) | 6 (11.32) | 2 (15.38) | |
| 30-34 | 30 (15.63) | 19 (17.59) | 2 (11.11) | 6 (11.32) | 3 (23.08) | |
| ≥35 | 86 (44.79) | 28 (25.93) | 15 (83.33) | 37 (69.81) | 6 (46.15) | |
| Body Mass Index | 34.76 (10.16) | 30.63 (7.54) | 41.49 (7.45) | 40.29 (11.07) | 37.22 (12.76) | <0.0001 |
| [mean (SD)] | | | | | | |
| (n = 192) | | | | | | |
| Menopause | | | | | | |
| status [n (%)] | | | | | | |
| Premenopausal | 108 (56.84) | 89 (82.41) | 6 (33.33) | 12 (23.53) | 1 (7.69) | <0.0001 |
| Postmenopausal | 82 (43.16) | 19 (17.59) | 12 (66.67) | 39 (76.47) | 12 (92.31) | |
| Education (n (%)) | | | | | | |
| (n = 175) | | | | | | |
| Less than high | 10 (5.71) | 3 (2.86) | 2 (14.29) | 4 (8.89) | 1 (9.09) | 0.36 |
| school | | | | | | |
| High school | 38 (21.71) | 20 (19.05) | 6 (42.86) | 10 (22.22) | 2 (18.18) | |
| diploma or GED | | | | | | |
| Some college | 42 (24.00) | 24 (22.86) | 4 (28.57) | 12 (26.67) | 2 (18.18) | |
| Association | 35 (20.00) | 22 (20.95) | 1 (7.14) | 9 (20.00) | 3 (27.27) | |
| degree or | | | | | | |
| technical | | | | | | |
| certification | | | | | | |
| Bachelor's degree | 30 (17.14) | 22 (20.95) | 0 (0.00) | 5 (11.11) | 3 (27.27) | |
| Master's/doctor's | 20 (11.43) | 14 (13.33) | 1 (7.14) | 5 (11.11) | 0 (0.00) | |
| degree | | | | | | |
| Income (n (%)) | | | | | | |
| (n = 168) | | | | | | |
| <10,000 | 8 (4.60) | 3 (3.03) | 0 (0.00) | 5 (11.36) | 0 (0.00) | 0.002 |
| 10,000-25,000 | 26 (14.94) | 10 (10.10) | 6 (42.86) | 7 (15.91) | 3 (27.27) | |
| 25,000-50,000 | 39 (22.41) | 13 (13.13) | 3 (21.43) | 16 (36.36) | 4 (36.36) | |
| 50,000-75,000 | 32 (18.39) | 23 (23.23) | 2 (14.29) | 6 (13.64) | 1 (9.09) | |
| 75,000-100,000 | 18 (10.34) | 15 (15.15) | 1 (7.14) | 1 (2.27) | 1 (9.09) | |
| >100,000 | 32 (18.39) | 24 (24.24) | 1 (7.14) | 3 (6.82) | 2 (18.18) | |
| Don't know/ | 19 (10.32) | 11 (11.11) | 1 (7.14) | 6 (13.64) | 0 (0.00) | |
| refused | | | | | | |
| Marital Status | | | | | | |
| (n (%)) (n = 191) | | | | | | |
| Single/divorced/ | 85 (44.50) | 41 (37.96) | 8 (44.44) | 30 (57.69) | 6 (46.15) | 0.41 |
| widowed | | | | | | |
| Married | 96 (50.26) | 60 (55.56) | 8 (44.44) | 21 (40.38) | 7 (53.85) | |
| Cohabitating | 6 (3.14) | 5 (4.63) | 1 (5.56) | 0 (0.00) | 0 (0.00) | |
| Other | 4 (2.09) | 2 (11.85) | 1 (5.56) | 1 (1.54) | 0 (0.00) | |
| Sexual | | | | | | |
| Orientation | | | | | | |
| (n (%)) (n = 171) | | | | | | |
| Heterosexual | 164 (95.91) | 93 (93.00) | 14 (100.00) | 44 (10.00) | 13 (100.00) | 0.52 |
| Bisexual | 2 (1.17) | 2 (2.00) | 0 (0.00) | 0 (0.00) | 0 (0.00) | |
| Homosexual | 5 (2.92) | 5 (5.00) | 0 (0.00) | 0 (0.00) | 0 (0.00) | |
| Employment | | | | | | |
| Status (n (%)) | | | | | | |
| (n = 174) | | | | | | |
| Employed | 100 (57.47) | 75 (72.82) | 7 (50.00) | 14 (31.11) | 4 (33.33) | <0.0001 |
| Unemployed | 74 (42.53)) | 28 (27.18) | 7 (50.00) | 31 (68.89) | 8 (66.67) | |
| Contraceptive | | | | | | |
| use in past 6 | | | | | | |
| months (n (%)) | | | | | | |
| Birth control pill | | | | | | |
| (n = 127) | | | | | | |
| Yes | 15 (11.81) | 12 (16.00) | 0 (0.00) | 3 (9.38) | 0 (0.00) | 0.25 |
| No | 112 (88.19) | 63 (84.00) | 11 (100.00) | 29 (90.63) | 9 (100.00) | |
| Depo-Provera ® | | | | | | |
| (n = 74) | | | | | | |
| Yes | 1 (1.35) | 0 (0.00) | 0 (0.00) | 0 (0.00) | 1 (20.00) | 0.07 |
| No | 73 (98.65) | 45 (100.00) | 9 (100.00) | 15 (100.00) | 4 (80.00) | |
| Paragard ® (n = 64) | | | | | | |
| Yes | 2 (3.13) | 1 (2.50) | 0 (0.00) | 1 (7.14) | 0 (0.00) | 0.61 |
| No | 62 (96.88) | 39 (97.50 | 6 (100.00) | 13 (92.86) | 4 (100.00) | |
| Hormone IUD | | | | | | |
| (n = 68) | | | | | | |
| Yes | 10 (14.71) | 9 (20.43) | 0 (0.00) | 0 (0.00) | 1 (20.00) | 0.13 |
| No | 58 (85.29) | 33 (78.57) | 7 (100.00) | 14 (100.00) | 4 (80.00) | |
| Type of | | | | | | |
| contraceptives | | | | | | |
| (n (%)) | | | | | | |
| Hormonal (n = 138) | | | | | | |
| Yes | 36 (26.09) | 26 (32.10) | 0 (0.00) | 5 (14.71) | 5 (45.45) | 0.01 |
| No | 102 (73.91) | 55 (67.90) | 12 (100.00) | 29 (85.29) | 6 (54.55) | |
| Non-hormonal | | | | | | |
| (n = 64) | | | | | | |
| Yes | 2 (3.13) | 1 (2.50) | 0 (0.00) | 1 (7.14) | 0 (0.00) | 0.62 |
| No | 62 (96.88) | 39 (97.50 | 6 (100.00) | 13 (92.86) | 4 (100.00) | |
| Surgical | | | | | | |
| contraception | | | | | | |
| (n (%)) (n = 100) | | | | | | |
| Tubal ligation | 40 (40.00) | 25 (40.98) | 3 (37.50) | 10 (41.67) | 2 (20.00) | 0.73 |
| Essure ® | 5 (5.00) | 5 (8.20) | 0 (0.00) | 0 (0.00) | 0 (0.00) | |
| Both ovaries | 1 1.00) | 1 (1.64) | 0 (0.00) | 0 (0.00) | 0 (0.00) | |
| removed | | | | | | |
| Tubes and | 3 (3.00) | 2 (3.28) | 1 (12.50) | 0 (0.00) | 0 (0.0) | |
| ovaries removed | | | | | | |
| None | 51 (51.00) | 19 (61.29) | 4 (50.00) | 14 (58.33) | 8 (80.00) | |
| Hormone | | | | | | |
| replacement | | | | | | |
| therapy (n (%)) | | | | | | |
| Hormone receptor | | | | | | |
| modulator, | | | | | | |
| agonist or | | | | | | |
| antagonist (n = 64) | | | | | | |
| Yes | 2 (3.13) | 2 (5.13) | 0 (0.00) | 0 (0.00) | 0 (0.00) | 0.99 |
| No | 62 (96.88) | 37 (94.87) | 6 (100.00) | 14 (100.00) | 5 (100.00) | |
| Hormone | | | | | | |
| treatment | | | | | | |
| (estrogen, estrogen/ | | | | | | |
| progesterone) | | | | | | |
| (n = 80) | | | | | | |
| Yes | 15 (18.75) | 10 (20.41) | 0 (0.00) | 2 (11.11) | 3 (42.86) | 0.11 |
| No | 65 (81.25)) | 39 (79.59) | 7 (100.00) | 16 (88.89) | 4 (57.14) | |
| Parity (n (%)) | | | | | | |
| (n = 191) | | | | | | |
| 0 | 44 (23.04) | 22 (20.56) | 4 (22.22) | 15 (28.30) | 3 (23.08) | 0.3 |
| 1 | 24 (12.57) | 9 (8.41) | 4 (22.22) | 8 (15.09) | 3 (23.08) | |
| 2 | 42 (21.99) | 23 (21.50) | 7 (38.89) | 10 (18.87) | 2 (15.38) | |
| 3 | 44 (23.04) | 27 (25.23) | 2 (11.11) | 13 (24.53) | 2 (15.38) | |
| 4+ | 37 (19.37) | 26 (24.30) | 1 (5.56) | 7 (13.21) | 3 (23.08) | |
| Heavy periods | | | | | | |
| (n (%)) (n = 150) | | | | | | |
| Light | 9 (6.00) | 5 (5.32) | 0 (0.00) | 2 (5.88) | 2 (20.00) | 0.01 |
| Moderate | 48 (32.00) | 21 (22.34) | 6 (50.00) | 17 (50.0) | 4 (40.00) | |
| Heavy | 93 (62.00) | 68 (72.34) | 6 (50.00) | 15 (44.12) | 4 (40.00) | |
| Douching (n (%)) | | | | | | |
| (n = 161) | | | | | | |
| Yes | 26 (16.15) | 15 (15.96) | 3 (21.43) | 7 (16.28) | 1 (10.00) | 0.9 |
| No | 135 (83.85) | 79 (84.04) | 11 (78.57) | 36 (83.72) | 9 (90.00) | |
| Dilation and | | | | | | |
| curettage (n (%)) | | | | | | |
| (n = 192) | | | | | | |
| Yes | 58 (30.21) | 19 (17.59) | 7 (38.89) | 25 (47.17) | 7 (53.85) | 0.0002 |
| No | 134 (69.79) | 89 (82.41) | 11 (61.11) | 28 (52.83) | 6 (46.15) | |
| Uterine | | | | | | |
| manipulator type | | | | | | |
| used (n (%)) | | | | | | |
| (n = 185) | | | | | | |
| Fornisee ® | 63 (34.05) | 61 (58.65) | 2 (12.50) | 0 (0.00) | 0 (0.00) | <0.0001 |
| Sacrocervicopexy | 0 (0.00) | 0 (0.00) | 0 (0.00) | 0 (0.00) | 0 (0.00) | |
| Delineator ™ | 77 (41.62) | 31 (29.81) | 4 (25.00) | 36 (67.92) | 6 (50.00) | |
| VCare ® | 21 (11.35) | 5 (4.81) | 4 (25.00) | 7 (13.21) | 5 (41.67)) | |
| RUMI ® | 23 (12.43) | 6 (5.77) | 6 (37.50) | 10 (18.87) | 1 (8.33) | |
| Sponge stick | 1 (0.54) | 1 (0.96) | 0 (0.00) | 0 (0.00) | 0 (0.00) | |
| Chronic pelvic | | | | | | |
| pain hx (n (%)) | | | | | | |
| (n = 155) | | | | | | |
| Yes | 64 (41.29) | 50 (54.95) | 2 (15.38) | 10 (25.00) | 2 (18.18) | 0.001 |
| No | 91 (58.71) | 41 (45.05) | 11 (84.62) | 30 (75.00) | 9 (81.82) | |
| Endometriosis | | | | | | |
| hx (n (%)) | | | | | | |
| (n = 162) | | | | | | |
| Yes | 43 (26.54) | 28 (29.47) | 2 (14.29) | 12 (28.57) | 1 (9.09) | 0.35 |
| No | 119 (73.46) | 67 (70.53) | 12 (85.71) | 30 (71.43) | 10 (90.91) | |
| PCOS hx (n (%)) | | | | | | |
| (n = 154) | | | | | | |
| Yes | 16 (10.39) | 10 (11.36) | 2 (13.33) | 4 (10.00) | 0 (0.00 | 0.68 |
| No | 138 (89.61) | 78 (88.64) | 13 (86.67) | 36 (90.00) | 11 (100.00) | |
| Diabetes (n (%)) | | | | | | |
| (n = 192) | | | | | | |
| Yes | 48 (25.00) | 22 (20.37) | 5 (27.78) | 21 (39.62) | 0 (0.00) | 0.006 |
| No | 144 (75.00) | 86 (79.63) | 13 (72.22) | 32 (60.38) | 13 (100.00) | |
| Hypertension | | | | | | |
| (n (%)) (n = 192) | | | | | | |
| Yes | 65 (33.85) | 25 (23.15) | 6 (33.33) | 27 (50.94) | 7 (53.85) | 0.002 |
| No | 127 (66.15) | 83 (76.85) | 12 (66.67) | 26 (49.06) | 6 (46.15) | |
| Antibiotics | | | | | | |
| (recent use) | | | | | | |
| (n (%)) (n = 161) | | | | | | |
| Yes | 41 (25.47) | 23 (24.21) | 4 (26.67) | 10 (24.39) | 4 (40.00) | 0.75 |
| No | 120 (74.53) | 72 (75.79) | 11 (73.33) | 31 (75.16) | 6 (60.00) | |
| Alcohol use | | | | | | |
| (n (%)) (n = 175) | | | | | | |
| Yes | 72 (41.41) | 50 (49.50) | 7 (43.75) | 11 (24.44) | 4 (30.77) | 0.11 |
| No | 94 (69.23) | 47 (46.53) | 8 (50.00) | 30 (66.67) | 9 (69.23) | |
| Quit | 9 (5.14) | 4 (3.96) | 1 (6.25) | 4 (8.89) | 0 (0.00) | |
| Tobacco use | | | | | | |
| (n (%)) (n = 184) | | | | | | |
| Yes | 19 (10.33) | 14 (13.46) | 1 (5.56) | 3 (6.12) | 1 (7.69) | 0.15 |
| No | 55 (29.89) | 34 (32.69) | 4 (22.22) | 13 (26.53) | 4 (30.77) | |
| Never | 89 (48.37) | 47 (45.19) | 12 (66.67) | 22 (44.90) | 8 (61.54) | |
| Quit | 21 (11.41) | 9 (8.65) | 1 (5.56) | 11 (22.45) | 0 (0.00) | |

Abbreviations: body mass index (BMI), endometrial cancer (EC), endometrial endometrioid carcinoma (EEC), general educational development (GED), medical history (hx), polycystic ovary syndrome (PCOS), standard deviation (SD).

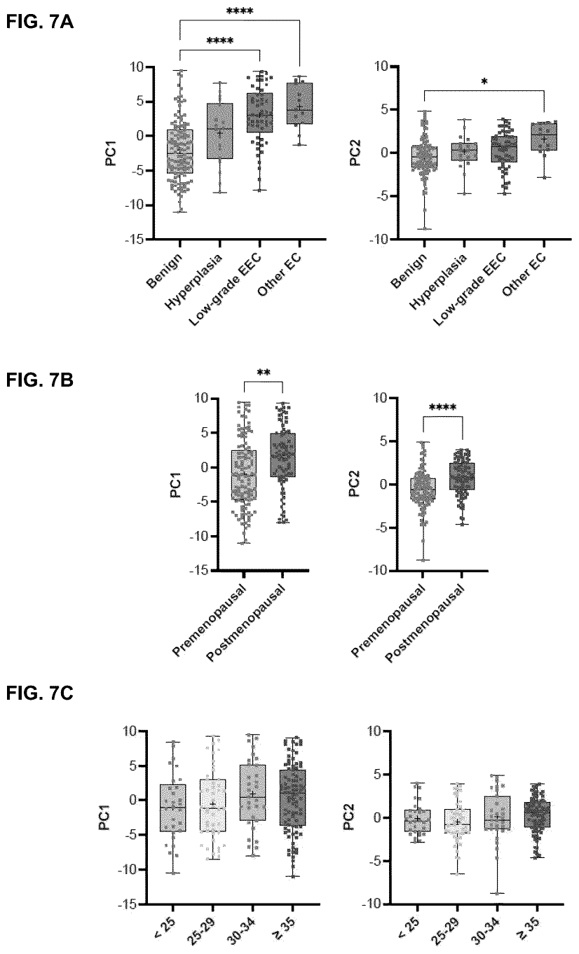

Cervicovaginal protein profiles. CVL samples were collected from all participants (N=192) and used to quantify 72 soluble proteins, including cytokines, chemokines, growth factors, apoptosis-related proteins, hormones, circulating tumor markers, and immune checkpoint proteins (see above). All tested proteins were measurable in CVL. The principal component analysis (PCA) was used to illustrate global protein profiles of individual samples (FIGS. 1A, 1B, and 1C). PCA reduces the dimensionality of large datasets while preserving the maximum information amount. A set of variables (i.e., cervicovaginal levels of 72 protein) were transformed to a smaller number of principal components that account for most of the variance. We utilized the first two principal components (PC1 and PC2), which explained 41.6% of the variance in the data. A multivariate analysis of variance revealed significant differences among the disease groups (P<0.0001) (FIG. 1A). The analysis also demonstrated that global protein profiles significantly differ between premenopausal and postmenopausal women (P<0.0001) (FIG. 1B) but not between women varying by BMI (P=0.33) (FIG. 1C). Subsequent pairwise comparisons showed that PC1 significantly varied between the disease groups (low-grade EEC vs. benign, P<0.0001; other EC vs. benign, P<0.0001) and menopausal status (P=0.001) but did not vary among the BMI categories (FIGS. 7A, 7B, and 7C). PC2 was also significantly different between the disease groups (other EC vs. benign, P=0.02) and the menopausal status categories (P<0.0001)

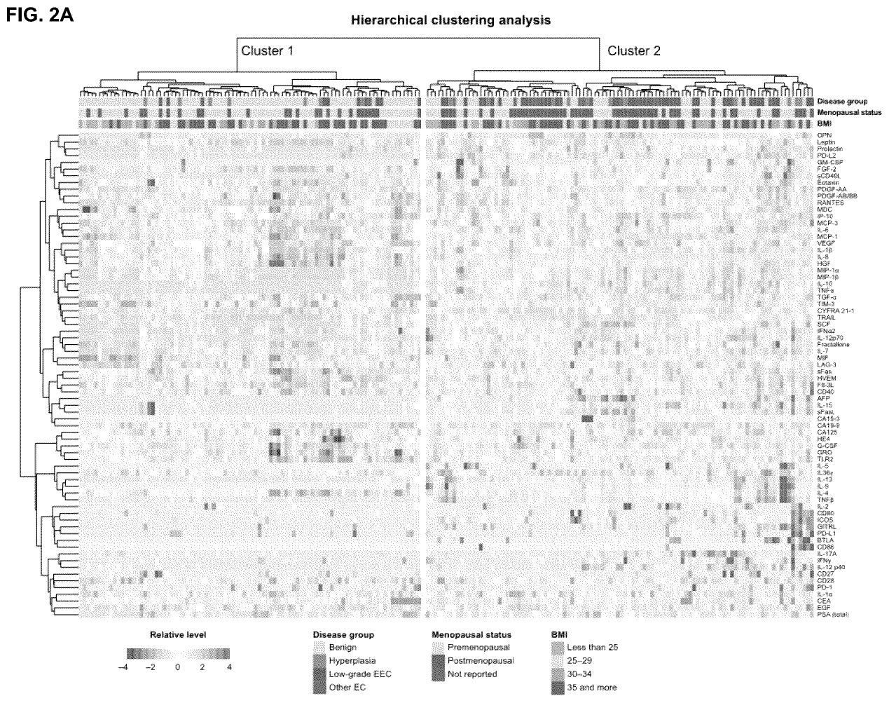

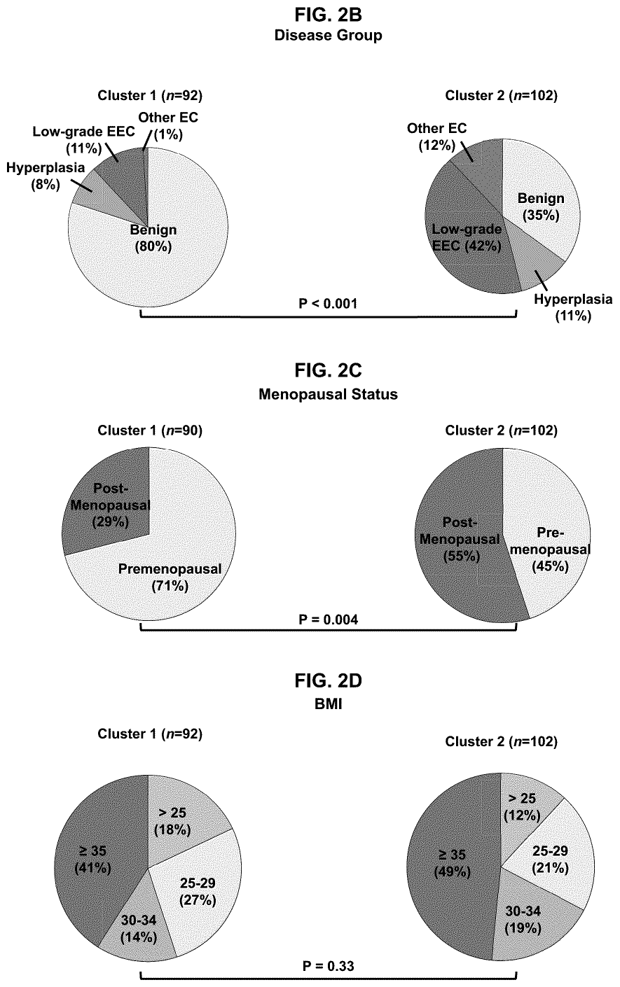

To further analyze global protein profiles, an unsupervised hierarchical clustering analysis was performed (FIGS. 2A, 2B, 2C, and 2D). A heatmap with a dendrogram revealed two distinct clusters. To characterize these clusters, the metadata was plotted (such as disease group, menopausal status, and BMI) related to individual samples above the heatmap (FIG. 2A) and analyzed statistical differences among these patient-related factors between the clusters. The distribution of disease groups significantly varied (P<0.0001) between the clusters. Cluster 1 was predominated by samples from the benign group (80%), whereas Cluster 2 had the highest proportion of samples from women diagnosed with EC (64%) (FIG. 2B). The menopausal status also significantly varied between the clusters (P=0.0004) (FIG. 2C); however, there were no differences in the distribution of BMI categories (P=0.33) (FIG. 2D). Overall, the data reduction analyses revealed that women with benign conditions and women with EC exhibit distinctive cervicovaginal protein profiles.

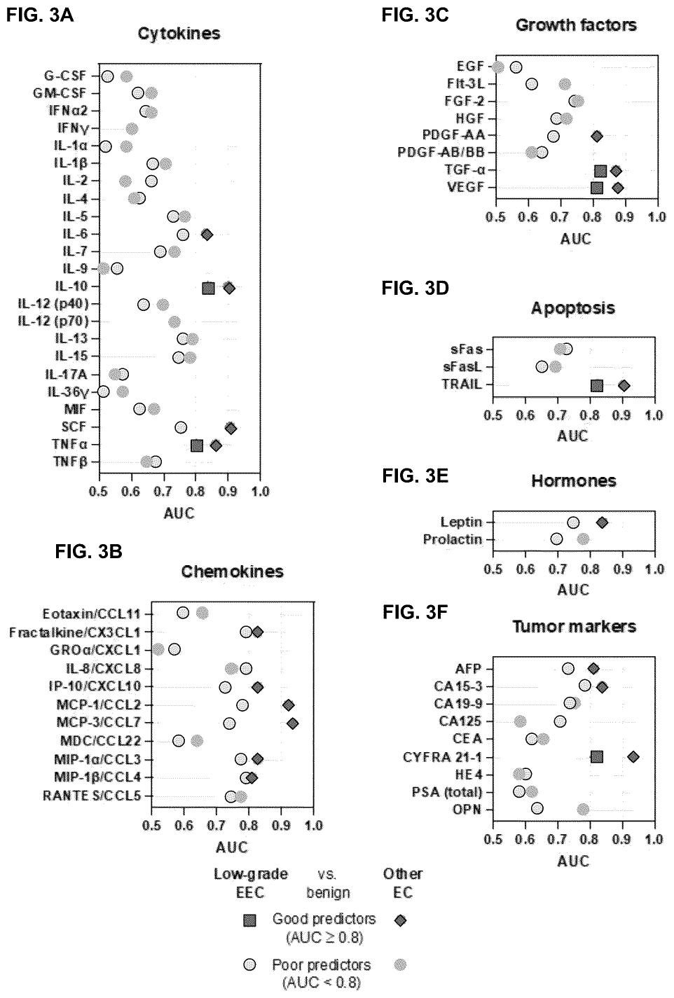

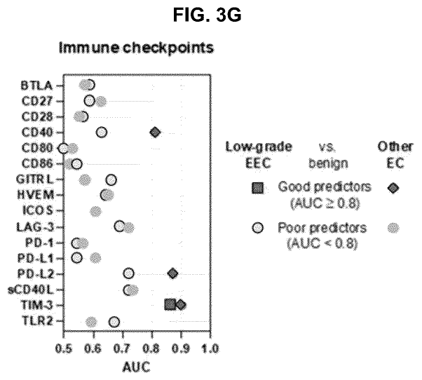

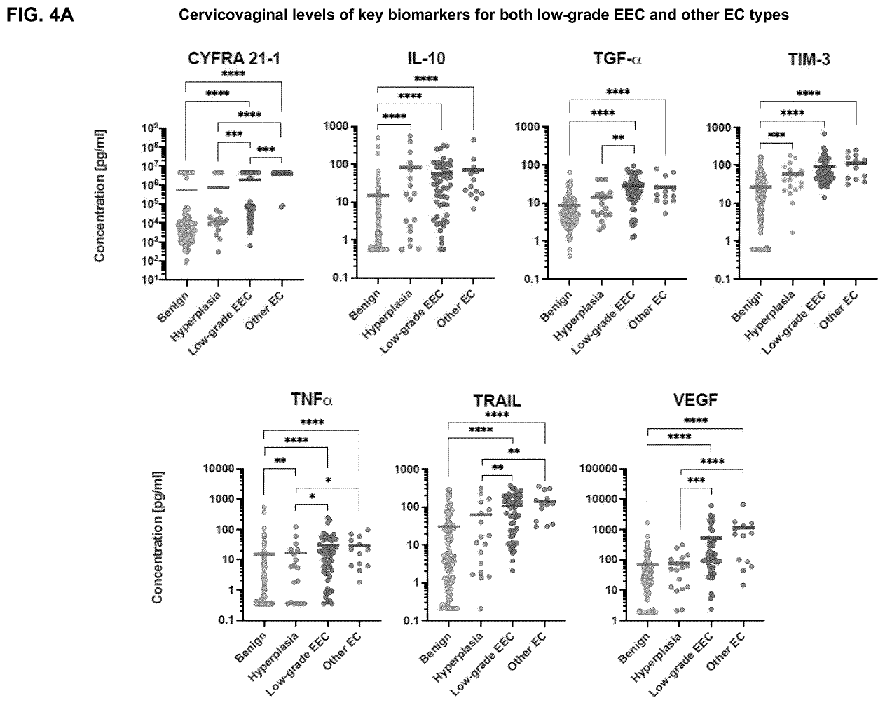

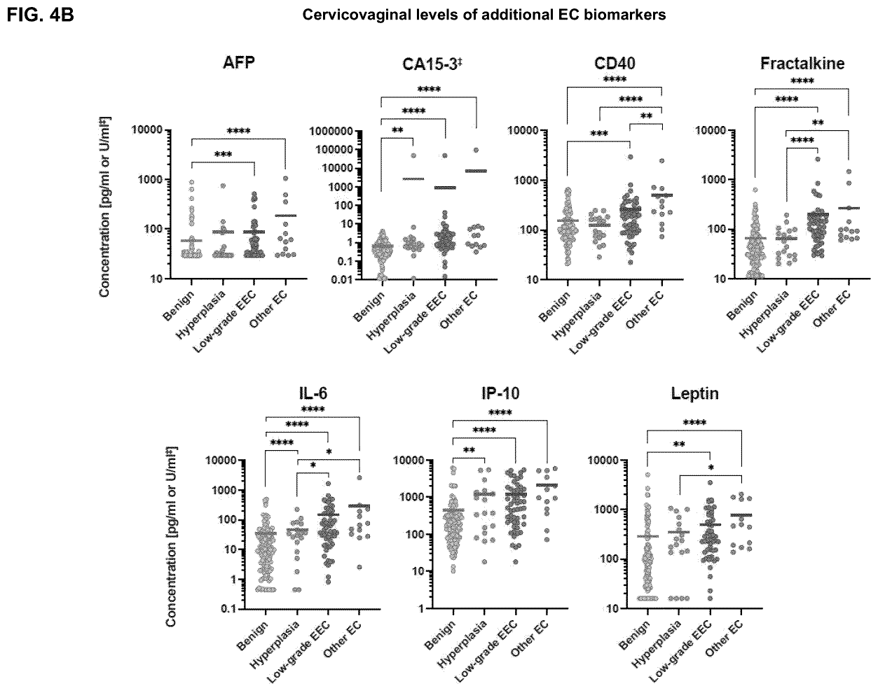

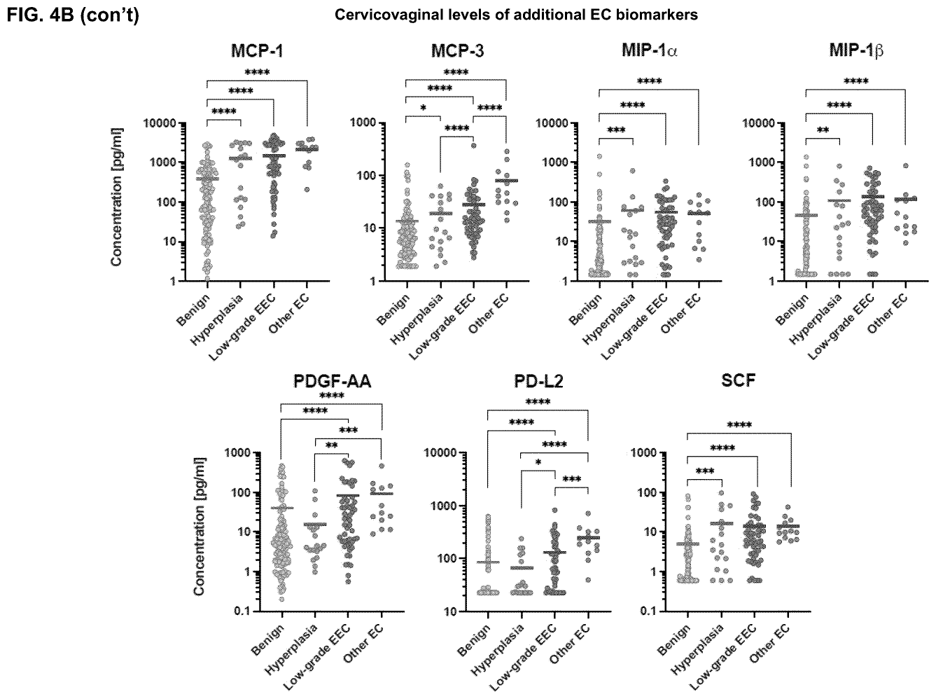

Cervicovaginal biomarkers for detection of EC. Next, the levels of proteins measured in CVL samples were compared among the disease groups. Since age and BMI were significantly different among the disease groups (Table 1), P values were adjusted for these factors. Fifty-four out of 72 protein targets were significantly elevated in women with low-grade EEC compared to benign (P ranging from 0.05 to <0.0001) (Table 2). Twenty targets were significantly elevated in endometrial hyperplasia (P ranging from 0.02 to <0.0001), and 40 targets were elevated in other EC subtypes (P ranging from 0.05 to <0.0001) (Table 2). To identify biomarkers with high sensitivity and specificity, a receiver operating characteristics (ROC) analysis was performed. Proteins with the area under the curve (AUC), which shows the relationship between sensitivity and specificity, greater than or equal to 0.8 were considered as good discriminators. The analysis comparing low-grade EEC or other EC to benign conditions revealed seven proteins with good discriminatory properties for both EC subtypes: TIM-3 (AUC 0.86 and 0.90), IL-10 (AUC 0.84 and 0.90), TRAIL (AUC 0.82 and 0.90), TGF-α (AUC 0.82 and 0.87), CYFRA 21-1 (AUC 0.82 and 0.93), VEGF (AUC 0.81 and 0.88), and TNFα (AUC 0.80 and 0.86) (FIG. 3A-3G). Notably, all seven proteins reached higher AUC values for the other EC group than for the low-grade EEC group. In addition, the analysis revealed 14 additional proteins (including cytokines: IL-6 and SCF, chemokines: fractalkine, IP-10, MCP-1, MCP-3, MIP-1α, and MIP-1β, a growth factor PDGF-AA, hormone leptin, tumor markers: AFP, CA15-3, and immune checkpoint proteins: CD40 and PD-L2) with good discriminatory properties for other EC subtype group, but not low-grade EEC when compared to benign conditions (FIG. 3A-3G). When the cervicovaginal levels of key biomarkers for both EC subtypes, identified in the ROC analysis, were compared among the disease groups, all seven proteins (CYFRA 21-1, IL-10, TGF-α, TIM-3, TNFα, TRAIL, and VEGF were significantly (P<0.0001) elevated in both low-grade EEC and other EC groups when compared to benign conditions (FIG. 4A). Of those, only CYFRA-21 significantly (P=0.001) differed in mean levels between low-grade EEC and other EC subtypes. In addition, IL-10 and TIM-3 levels were also elevated in endometrial hyperplasia patients compared to the benign group (P<0.0001 and P=0.006, respectively). For the additional 14 biomarkers for other EC, identified in ROC analysis, cervicovaginal levels were also significantly elevated in both EC groups (low-grade EEC and other EC subtypes) when compared to benign conditions (P ranging from 0.001 to <0.0001). Three out of 14 biomarkers: CD40, MCP-3, and PD-L2, had higher cervicovaginal levels in other EC than the low-grade EEC group (P ranging from 0.008 to <0.0001). Notably, MCP-3 was also identified as a good discriminator (AUC =0.832) between other EC subtypes and low-grade EEC in the subsequent ROC analysis. In addition, levels of eight proteins, mostly chemokines, i.e., CA15-3, IL-6, IP-10, MCP-1, MCP-3, MIP-1α, MIP-1β were significantly (P ranging from 0.01 to <0.0001) elevated in the endometrial hyperplasia group when compared to patients with benign conditions. Overall, these analyses identified biomarker candidates for detecting EC using CVL sampling.

TABLE 2
The significance of difference between protein levels among the disease groups.
P values were calculated using a linear mixed effects model. If the overall difference was
significant (P < 0.05), paired tests were performed with Bonferroni adjustment. Comparisons were
adjusted for age and BMI by including these variables as predictors in the models.

| | | | | | | | Adjusted for age, BMI | | | | |
|---|---|---|---|---|---|---|---|---|---|---|---|
| | | | | P value | | | | | P value | | |
| Target | Group | Mean | SE | overall | paired | | Mean | SE | overall | paired | |
| AFP | | | | | | | | | | | |
| | Benign | 58.49 | 9.47 | <0.0001 | (1) vs. | <0.0001 | 66.88 | 10.24 | <0.0001 | (1) vs. | 0.0005 |
| | | | | | (3) | | | | | (3) | |
| | Hyperplasia | 88.14 | 23.20 | | (1) vs. | <0.0001 | 103.43 | 25.00 | | (1) vs. | <0.0001 |
| | | | | | (4) | | | | | (4) | |
| | Low-grade | 87.85 | 13.52 | | (2) vs. | 0.02 | 97.78 | 16.18 | | | |
| | EEC | | | | (4) | | | | | | |
| | Other | 187.27 | 27.30 | | | | 185.74 | 28.06 | | | |
| | EC | | | | | | | | | | |
| BTLA | | | | | | | | | | | |
| | Benign | 48.12 | 21.77 | 0.06 | | | 52.42 | 23.75 | 0.02 | (1) vs. | 0.02 |
| | | | | | | | | | | (3) | |
| | Hyperplasia | 102.07 | 53.32 | | | | 141.02 | 57.97 | | | |
| | Low-grade | 101.00 | 31.07 | | | | 123.96 | 37.53 | | | |
| | EEC | | | | | | | | | | |
| | Other | 33.13 | 62.74 | | | | 39.52 | 65.07 | | | |
| | EC | | | | | | | | | | |
| CA15-3 | | | | | | | | | | | |
| | Benign | 51.42 | 699.44 | <0.0001 | (1) vs. | 0.003 | 225.86 | 748.64 | <0.0001 | (1) vs. | 0.003 |
| | | | | | (2) | | | | | (2) | |
| | Hyperplasia | 2687.33 | 1604.05 | | (1) vs. | <0.0001 | 2739.77 | 1743.40 | | (1) vs | <0.0001 |
| | | | | | (3) | | | | | (3) | |
| | Low-grade | 950.14 | 960.05 | | (1) vs. | <0.0001 | 786.06 | 1144.21 | | (1) vs. | <0.0001 |
| | EEC | | | | (4) | | | | | (4) | |
| | Other | 7343.78 | 1880.08 | | (2) vs. | 0.02 | 6995.39 | 1953.11 | | | |
| | EC | | | | (4) | | | | | | |
| CA19-9 | | | | | | | | | | | |
| | Benign | 36233.00 | 9943.92 | <0.0001 | (1) vs. | <0.0001 | 49649.00 | 10745.00 | <0.0001 | (1) vs. | <0.0001 |
| | | | | | (2) | | | | | (2) | |
| | Hyperplasia | 201792.00 | 24358.00 | | (1) vs. | <0.0001 | 196314.00 | 26226.00 | | (1) vs. | <0.0001 |
| | | | | | (3) | | | | | (3) | |
| | Low-grade | 160993.00 | 14195.00 | | (1) vs. | 0.001 | 148993.00 | 16980.00 | | | |
| | EEC | | | | (4) | | | | | | |
| | Other | 83596.00 | 28661.00 | | | | 67422.00 | 29441.00 | | | |
| | EC | | | | | | | | | | |
| CA125 | | | | | | | | | | | |
| | Benign | 1551.21 | 419.20 | <0.0001 | (1) vs. | <0.0001 | 1539.70 | 456.93 | <0.0001 | (1) vs. | 0.02 |
| | | | | | (3) | | | | | (2) | |
| | Hyperplasia | 2291.78 | 1026.83 | | | | 2422.55 | 1115.22 | | (1) vs. | <0.0001 |
| | | | | | | | | | | (3) | |
| | Low-grade | 5720.41 | 598.41 | | | | 5730.09 | 722.06 | | | |
| | EEC | | | | | | | | | | |
| | Other | 3797.95 | 1208.27 | | | | 3685.16 | 1251.93 | | | |
| | EC | | | | | | | | | | |
| CD27 | | | | | | | | | | | |
| | Benign | 10.62 | 1.04 | 0.0004 | (1) vs. | 0.002 | 9.86 | 1.12 | 0.002 | (1) vs. | 0.004 |
| | | | | | (3) | | | | | (3) | |
| | Hyperplasia | 15.37 | 2.54 | | | | 16.38 | 2.74 | | (1) vs. | 0.05 |
| | | | | | | | | | | (4) | |
| | Low-grade | 16.54 | 1.48 | | | | 17.78 | 1.77 | | | |
| | EEC | | | | | | | | | | |
| | Other | 17.28 | 2.98 | | | | 18.34 | 3.08 | | | |
| | EC | | | | | | | | | | |
| CD28 | | | | | | | | | | | |
| | Benign | 594.67 | 44.69 | 0.04 | | | 636.63 | 47.90 | 0.99 | | |
| | Hyperplasia | 719.73 | 109.46 | | | | 565.97 | 116.90 | | | |
| | Low-grade | 641.38 | 63.79 | | | | 473.80 | 75.69 | | | |
| | EEC | | | | | | | | | | |
| | Other | 551.30 | 128.80 | | | | 423.68 | 131.23 | | | |
| | EC | | | | | | | | | | |
| CD40 | | | | | | | | | | | |
| | Benign | 154.08 | 19.54 | <0.0001 | (1) vs. | 0.0003 | 155.18 | 20.82 | <0.0001 | (1) vs. | 0.0002 |
| | | | | | (3) | | | | | (3) | |
| | Hyperplasia | 125.68 | 47.87 | | (1) vs. | <0.0001 | 194.15 | 50.81 | | (1) vs. | <0.0001 |
| | | | | | (4) | | | | | (4) | |
| | Low-grade | 260.17 | 27.90 | | (2) vs. | 0.02 | 306.83 | 32.89 | | (2) vs. | <0.0001 |
| | EEC | | | | (3) | | | | | (4) | |
| | Other | 499.86 | 56.33 | | (2) vs. | <0.0001 | 515.29 | 57.03 | | (3) vs. | 0.008 |
| | EC | | | | (4) | | | | | (4) | |
| | | | | | (3) vs. | 0.002 | | | | | |
| | | | | | (4) | | | | | | |
| CD80 | | | | | | | | | | | |
| | Benign | 8.78 | 1.47 | 0.4 | | | 10.97 | 1.57 | 0.48 | | |
| | Hyperplasia | 9.05 | 3.60 | | | | 9.35 | 3.83 | | | |
| | Low-grade | 6.81 | 2.10 | | | | 5.97 | 2.48 | | | |
| | EEC | | | | | | | | | | |
| | Other | 10.29 | 4.23 | | | | 7.77 | 4.30 | | | |
| | EC | | | | | | | | | | |
| CD86 | | | | | | | | | | | |
| | Benign | 34.42 | 2.60 | 0.29 | | | 34.21 | 2.82 | 0.1 | | |
| | Hyperplasia | 33.39 | 6.37 | | | | 37.74 | 6.88 | | | |
| | Low-grade | 37.12 | 3.71 | | | | 40.31 | 4.46 | | | |
| | EEC | | | | | | | | | | |
| | Other | 29.55 | 7.50 | | | | 30.61 | 7.73 | | | |
| | EC | | | | | | | | | | |
| CEA | | | | | | | | | | | |
| | Benign | 30780.00 | 3884.53 | 0.002 | (1) vs. | 0.01 | 23390.00 | 4109.60 | 0.68 | | |
| | | | | | (3) | | | | | | |
| | Hyperplasia | 5049.94 | 9515.11 | | | | 8237.76 | 10030.00 | | | |
| | Low-grade | 16078.00 | 5545.14 | | | | 22797.00 | 6494.17 | | | |
| | EEC | | | | | | | | | | |
| | Other | 5443.86 | 11196.00 | | | | 14135.00 | 11260.00 | | | |
| | EC | | | | | | | | | | |
| CYFRA-21 | | | | | | | | | | | |
| | Benign | 564758.00 | 119000.00 | <0.0001 | (1) vs. | <0.0001 | 620325.00 | 129912.00 | <0.0001 | (1) vs. | <0.0001 |
| | | | | | (3) | | | | | (3) | |
| | Hyperplasia | 768621.00 | 291490.00 | | (1) vs. | <0.0001 | 932348.00 | 317076.00 | | (1) vs. | <0.0001 |
| | | | | | (4) | | | | | (4) | |
| | Low-grade | 1910789.00 | 169872.00 | | (2) vs. | 0.0002 | 2002619.00 | 205293.00 | | (2) vs. | 0.001 |
| | EEC | | | | (3) | | | | | (3) | |
| | Other | 3854685.00 | 342996.00 | | (2) vs. | <0.0001 | 3843956.00 | 355945.00 | | (2) vs. | <0.0001 |
| | EC | | | | (4) | | | | | (4) | |
| | | | | | (3) vs. | 0.0002 | | | | (3) vs. | 0.001 |
| | | | | | (4) | | | | | (4) | |
| EGF | | | | | | | | | | | |
| | Benign | 27.55 | 3.84 | 0.26 | | | 24.96 | 4.15 | 0.13 | | |
| | Hyperplasia | 29.75 | 9.41 | | | | 27.03 | 10.13 | | | |
| | Low-grade | 32.89 | 5.49 | | | | 33.76 | 6.56 | | | |
| | EEC | | | | | | | | | | |
| | Other | 45.19 | 11.08 | | | | 47.59 | 11.37 | | | |
| | EC | | | | | | | | | | |
| Eotaxin | | | | | | | | | | | |
| | Benign | 12.43 | 1.08 | 0.1 | | | 11.63 | 1.16 | 0.02 | (1) vs. | 0.05 |
| | | | | | | | | | | (3) | |
| | Hyperplasia | 8.49 | 2.63 | | | | 12.00 | 2.84 | | | |
| | Low-grade | 9.78 | 1.53 | | | | 13.02 | 1.84 | | | |
| | EEC | | | | | | | | | | |
| | Other | 11.38 | 3.10 | | | | 13.83 | 3.18 | | | |
| | EC | | | | | | | | | | |
| FIt-3L | | | | | | | | | | | |
| | Benign | 11.46 | 0.81 | 0.001 | (1) vs. | 0.02 | 11.26 | 0.86 | 0.005 | (1) vs. | 0.03 |
| | | | | | (3) | | | | | (3) | |
| | Hyperplasia | 9.69 | 1.98 | | (1) vs. | 0.01 | 11.74 | 2.10 | | (1) vs. | 0.02 |
| | | | | | (4) | | | | | (4) | |
| | Low-grade | 14.06 | 1.15 | | (2) vs. | 0.05 | 15.16 | 1.36 | | | |
| | EEC | | | | (4) | | | | | | |
| | Other | 14.47 | 2.33 | | | | 14.82 | 2.36 | | | |
| | EC | | | | | | | | | | |
| FGF-2 | | | | | | | | | | | |
| | Benign | 64.72 | 15.19 | <0.0001 | (1) vs. | <0.0001 | 48.76 | 16.48 | <0.0001 | (1) vs. | <0.0001 |
| | | | | | (3) | | | | | (3) | |
| | Hyperplasia | 57.16 | 37.20 | | (1) vs. | 0.02 | 78.08 | 40.22 | | (1) vs. | 0.02 |
| | | | | | (4) | | | | | (4) | |
| | Low-grade | 51.83 | 21.68 | | | | 78.17 | 26.04 | | | |
| | EEC | | | | | | | | | | |
| | Other | 39.83 | 43.77 | | | | 65.31 | 45.15 | | | |
| | EC | | | | | | | | | | |
| Fractalkine | | | | | | | | | | | |
| | Benign | 64.97 | 15.56 | <0.0001 | (1) vs. | <0.0001 | 74.95 | 16.64 | <0.0001 | (1) vs. | <0.0001 |
| | | | | | (3) | | | | | (3) | |
| | Hyperplasia | 64.77 | 38.10 | | (1) vs. | <0.0001 | 107.42 | 40.60 | | (1) vs. | <0.0001 |
| | | | | | (4) | | | | | (4) | |
| | Low-grade | 198.62 | 22.21 | | (2) vs. | <0.0001 | 232.57 | 26.29 | | (2) vs. | <0.0001 |
| | EEC | | | | (3) | | | | | (3) | |
| | Other | 263.27 | 44.84 | | (2) vs. | 0.0001 | 272.27 | 45.58 | | (2) vs. | 0.003 |
| | EC | | | | (4) | | | | | (4) | |
| GITRL | | | | | | | | | | | |
| | Benign | 15.93 | 1.91 | <0.0001 | (1) vs. | 0.003 | 14.59 | 2.07 | <0.0001 | (1) vs. | 0.001 |
| | | | | | (2) | | | | | (2) | |
| | Hyperplasia | 26.75 | 4.68 | | (1) vs. | <0.0001 | 28.95 | 5.06 | | (1) vs. | <0.0001 |
| | | | | | (3) | | | | | (3) | |
| | Type 1 | 24.19 | 2.73 | | | | 27.78 | 3.28 | | | |
| | EMC | | | | | | | | | | |
| | Other | 19.95 | 5.51 | | | | 23.23 | 5.68 | | | |
| | EC | | | | | | | | | | |
| G-CSF | | | | | | | | | | | |
| | Benign | 599.72 | 53.97 | 0.7 | | | 509.45 | 57.43 | 0.05 | | |
| | Hyperplasia | 512.52 | 132.20 | | | | 706.14 | 140.17 | | | |
| | Low-grade | 608.49 | 77.04 | | | | 833.85 | 90.75 | | | |
| | EEC | | | | | | | | | | |
| | Other | 668.74 | 155.56 | | | | 870.92 | 157.35 | | | |
| | EC | | | | | | | | | | |
| GROα | | | | | | | | | | | |
| | Benign | 2416.27 | 252.36 | 0.06 | | | 2198.42 | 275.29 | 0.01 | (1) vs. | 0.01 |
| | | | | | | | | | | (3) | |
| | Hyperplasia | 3022.51 | 618.16 | | | | 3269.20 | 671.89 | | | |
| | Low-grade | 3140.40 | 360.24 | | | | 3532.05 | 435.02 | | | |
| | EEC | | | | | | | | | | |
| | Other | 2496.41 | 727.38 | | | | 2919.51 | 754.26 | | | |
| | EC | | | | | | | | | | |
| GM- | | | | | | | | | | | |
| CSF | | | | | | | | | | | |
| | Benign | 6.27 | 0.62 | 0.3 | | | 5.16 | 0.67 | 0.001 | (1) vs. | 0.004 |
| | | | | | | | | | | (3) | |
| | Hyperplasia | 7.62 | 1.52 | | | | 8.52 | 1.63 | | (1) vs. | 0.02 |
| | | | | | | | | | | (4) | |
| | Low-grade | 5.54 | 0.89 | | | | 7.10 | 1.05 | | | |
| | EEC | | | | | | | | | | |
| | Other | 6.14 | 1.79 | | | | 7.92 | 1.83 | | | |
| | EC | | | | | | | | | | |
| HE4 | | | | | | | | | | | |
| | Benign | 76402.00 | 5135.86 | 0.03 | (1) vs. | 0.05 | 73082.00 | 5574.53 | <0.0001 | (1) vs. | 0.002 |
| | | | | | (3) | | | | | (2) | |
| | Hyperplasia | 98337.00 | 12580.00 | | | | 108354.00 | 13606.00 | | (1) vs. | <0.0001 |
| | | | | | | | | | | (3) | |
| | Low-grade | 99968.00 | 7331.41 | | | | 112159.00 | 8809.12 | | (1) vs. | 0.03 |
| | EEC | | | | | | | | | (4) | |
| | Other | 81533.00 | 14803.00 | | | | 91338.00 | 15274.00 | | | |
| | EC | | | | | | | | | | |
| HGF | | | | | | | | | | | |
| | Benign | 286.32 | 24.75 | <0.0001 | (1) vs. | <0.0001 | 272.3 | 26.77 | <0.0001 | (1) vs. | <0.0001 |
| | | | | | (3) | | | | | (3) | |
| | Hyperplasia | 249.61 | 60.63 | | (1) vs. | 0.0003 | 320.88 | 65.34 | | (1) vs. | 0.001 |
| | | | | | (4) | | | | | (4) | |
| | Low-grade | 394.08 | 35.33 | | (2) vs. | 0.04 | 459.81 | 42.30 | | | |
| | EEC | | | | (3) | | | | | | |
| | Other | 508.12 | 71.34 | | (2) vs. | 0.04 | 551.19 | 73.35 | | | |
| | EC | | | | (4) | | | | | | |
| HVEM | | | | | | | | | | | |
| | Benign | 770.96 | 38.61 | <0.0001 | (1) vs. | 0.001 | 755.26 | 41.64 | <0.0001 | (1) vs. | 0.0001 |
| | | | | | (3) | | | | | (3) | |
| | Hyperplasia | 657.73 | 94.57 | | (2) vs. | 0.001 | 760.73 | 101.63 | | (2) vs. | 0.001 |
| | | | | | (3) | | | | | (3) | |
| | Low-grade | 1027.96 | 55.11 | | (2) vs. | 0.02 | 1109.14 | 65.80 | | | |
| | EEC | | | | (4) | | | | | | |
| | Other | 1002.30 | 111.28 | | | | 1046.22 | 114.09 | | | |
| | EC | | | | | | | | | | |
| ICOS | | | | | | | | | | | |
| | Benign | 65.50 | 20.94 | 0.01 | (1) vs. | 0.03 | 70.87 | 22.79 | 0.07 | | |
| | | | | | (3) | | | | | | |
| | Hyperplasia | 80.88 | 51.30 | | | | 91.82 | 55.62 | | | |
| | Low-grade | 53.75 | 29.90 | | | | 50.48 | 36.01 | | | |
| | EEC | | | | | | | | | | |
| | Other | 49.37 | 60.36 | | | | 35.70 | 62.44 | | | |
| | EC | | | | | | | | | | |
| IFNα2 | | | | | | | | | | | |
| | Benign | 6.60 | 0.67 | 0.003 | (1) vs. | 0.004 | 6.21 | 0.73 | 0.01 | (1) vs. | 0.0 |
| | | | | | (3) | | | | | (3) | |
| | Hyperplasia | 5.96 | 1.64 | | | | 6.77 | 1.78 | | | |
| | Low-grade | 6.81 | 0.95 | | | | 7.81 | 1.15 | | | |
| | EEC | | | | | | | | | | |
| | Other | 6.88 | 1.92 | | | | 7.77 | 2.00 | | | |
| | EC | | | | | | | | | | |
| IFNγ | | | | | | | | | | | |
| | Benign | 1.43 | 0.15 | 0.05 | | | 1.36 | 0.16 | 0.02 | | |
| | Hyperplasia | 1.63 | 0.37 | | | | 1.79 | 0.40 | | | |
| | Low-grade | 1.50 | 0.22 | | | | 1.80 | 0.26 | | | |
| | EEC | | | | | | | | | | |
| | Other | 2.05 | 0.44 | | | | 2.31 | 0.45 | | | |
| | EC | | | | | | | | | | |
| IL-1α | | | | | | | | | | | |
| | Benign | 211.22 | 23.38 | 0.33 | | | 172.71 | 24.61 | 0.09 | | |
| | Hyperplasia | 135.13 | 57.27 | | | | 209.17 | 60.05 | | | |
| | Low-grade | 138.78 | 33.38 | | | | 218.21 | 38.88 | | | |
| | EEC | | | | | | | | | | |
| | Other | 134.18 | 67.39 | | | | 200.90 | 67.42 | | | |
| | EC | | | | | | | | | | |
| IL-1β | | | | | | | | | | | |
| | Benign | 132.40 | 24.27 | <0.0001 | (1) vs. | <0.0001 | 83.93 | 25.60 | <0.0001 | (1) vs. | <0.0001 |
| | | | | | (3) | | | | | (3) | |
| | Hyperplasia | 143.50 | 59.46 | | (1) vs. | 0.002 | 211.93 | 62.48 | | (1) vs. | 0.0001 |
| | | | | | (4) | | | | | (4) | |
| | Low-grade | 180.99 | 34.65 | | | | 271.95 | 40.45 | | | |
| | EEC | | | | | | | | | | |
| | Other | 300.47 | 69.97 | | | | 388.27 | 70.14 | | | |
| | EC | | | | | | | | | | |
| L-2 | | | | | | | | | | | |
| | Benign | 1.03 | 0.27 | <0.0001 | (1) vs. | 0.0004 | 1.04 | 0.30 | <0.0001 | (1) vs. | 0.002 |
| | | | | | (2) | | | | | (2) | |
| | Hyperplasia | 4.09 | 0.66 | | (1) vs. | 0.0004 | 4.07 | 0.72 | | (1) vs | 0.001 |
| | | | | | (3) | | | | | (3) | |
| | Low-grade | 2.47 | 0.39 | | (2) vs. | 0.01 | 2.55 | 0.47 | | | |
| | EEC | | | | (4) | | | | | | |
| | Other | 0.67 | 0.78 | | | | 0.71 | 0.81 | | | |
| | EC | | | | | | | | | | |
| IL-4 | | | | | | | | | | | |
| | Benign | 63.40 | 12.28 | 0.03 | (1) vs. | 0.04 | 60.10 | 13.40 | 0.004 | (1) vs. | 0.003 |
| | | | | | (3) | | | | | (3) | |
| | Hyperplasia | 57.45 | 30.08 | | | | 72.21 | 32.70 | | | |
| | Low-grade | 47.86 | 17.53 | | | | 64.79 | 21.17 | | | |
| | EEC | | | | | | | | | | |
| | Other | 57.48 | 35.39 | | | | 69.03 | 36.70 | | | |
| | EC | | | | | | | | | | |
| IL-5 | | | | | | | | | | | |
| | Benign | 0.83 | 0.21 | <0.0001 | (1) vs. | <0.0001 | 0.78 | 0.23 | <0.0001 | (1) vs. | <0.0001 |
| | | | | | (3) | | | | | (3) | |
| | Hyperplasia | 0.70 | 0.51 | | (1) vs. | 0.0003 | 0.70 | 0.55 | | (1) vs. | 0.0001 |
| | | | | | (4) | | | | | (4) | |
| | Low-grade | 1.78 | 0.30 | | (2) vs. | 0.04 | 1.87 | 0.36 | | (2) vs. | 0.04 |
| | EEC | | | | (3) | | | | | (3) | |
| | Other | 1.64 | 0.60 | | (2) vs. | 0.01 | 1.76 | 0.62 | | (2) vs. | 0.01 |
| | EC | | | | (4) | | | | | (4) | |
| IL-6 | | | | | | | | | | | |
| | Benign | 35.39 | 16.20 | <0.0001 | (1) vs. | <0.0001 | 21.73 | 17.51 | <0.0001 | (1) vs. | <0.0001 |
| | | | | | (3) | | | | | (2) | |
| | Hyperplasia | 46.76 | 39.67 | | (1) vs. | <0.0001 | 89.73 | 42.74 | | (1) vs | <0.0001 |
| | | | | | (4) | | | | | (3) | |
| | Low-grade | 148.25 | 23.12 | | (2) vs. | 0.04 | 186.15 | 27.67 | | (1) vs. | <0.0001 |
| | EEC | | | | (3) | | | | | (4) | |
| | Other | 294.10 | 46.68 | | (2) vs. | 0.01 | 322.86 | 47.97 | | (2) vs. | 0.03 |
| | EC | | | | (4) | | | | | (3) | |
| | | | | | | | | | | (2) vs. | 0.02 |
| | | | | | | | | | | (4) | |
| IL-7 | | | | | | | | | | | |
| | Benign | 3.31 | 0.25 | <0.0001 | (1) vs. | <0.0001 | 3.14 | 0.27 | <0.0001 | (1) vs. | <0.0001 |
| | | | | | (3) | | | | | (3) | |
| | Hyperplasia | 2.95 | 0.61 | | (1) vs. | 0.001 | 3.70 | 0.66 | | (1) vs. | <0.0001 |
| | | | | | (4) | | | | | (4) | |
| | Low-grade | 5.12 | 0.35 | | (2) vs. | 0.01 | 5.88 | 0.42 | | (2) vs. | 0.01 |
| | EEC | | | | (3) | | | | | (3) | |
| | Other | 4.98 | 0.72 | | (2) vs. | 0.03 | 5.55 | 0.74 | | (2) vs. | 0.03 |
| | EC | | | | (4) | | | | | (4) | |
| IL-8 | | | | | | | | | | | |
| | Benign | 1492.87 | 110.38 | <0.0001 | (1) vs. | 0.006 | 1598.85 | 119.50 | <0.0001 | (1) vs. | 0.004 |
| | | | | | (2) | | | | | (2) | |
| | Hyperplasia | 2479.45 | 270.38 | | (1) vs. | <0.0001 | 2649.44 | 291.66 | | (1) vs. | <0.0001 |
| | | | | | (3) | | | | | (3) | |
| | Low-grade | 3371.17 | 157.57 | | (1) vs. | <0.0001 | 3411.08 | 188.83 | | (1) vs. | 0.001 |
| | EEC | | | | (4) | | | | | (4) | |
| | Other | 2863.78 | 318.16 | | | | 2769.04 | 327.41 | | | |
| | EC | | | | | | | | | | |
| IL-9 | | | | | | | | | | | |
| | Benign | 1.36 | 0.16 | 0.72 | | | 1.28 | 0.18 | 0.2 | | |
| | Hyperplasia | 1.42 | 0.40 | | | | 1.65 | 0.44 | | | |
| | Low-grade | 1.11 | 0.23 | | | | 1.39 | 0.28 | | | |
| | EEC | | | | | | | | | | |
| | Other | 1.05 | 0.47 | | | | 1.27 | 0.49 | | | |
| | EC | | | | | | | | | | |
| IL-10 | | | | | | | | | | | |
| | Benign | 15.01 | 5.43 | <0.0001 | (1) vs. | <0.0001 | 13.09 | 5.91 | <0.0001 | (1) vs. | <0.0001 |
| | | | | | (2) | | | | | (2) | |
| | Hyperplasia | 80.86 | 13.31 | | (1) vs. | <0.0001 | 87.44 | 14.43 | | (1) vs. | <0.0001 |
| | | | | | (3) | | | | | (3) | |
| | Low-grade | 57.00 | 7.76 | | (1) vs. | <0.0001 | 66.28 | 9.35 | | (1) vs. | <0.0001 |
| | EEC | | | | (4) | | | | | (4) | |
| | Other | 71.15 | 15.66 | | | | 79.16 | 16.20 | | | |
| | EC | | | | | | | | | | |
| IL-12 | | | | | | | | | | | |
| (p40) | | | | | | | | | | | |
| | Benign | 5.20 | 0.23 | 0.0001 | (1) vs. | 0.001 | 5.22 | 0.25 | 0.0003 | (1) vs. | 0.002 |
| | | | | | (3) | | | | | (3) | |
| | Hyperplasia | 5.09 | 0.56 | | (1) vs. | 0.02 | 5.40 | 0.61 | | (1) vs. | 0.02 |
| | | | | | (4) | | | | | (4) | |
| | Low-grade | 6.30 | 0.33 | | | | 6.59 | 0.39 | | | |
| | EEC | | | | | | | | | | |
| | Other | 6.98 | 0.66 | | | | 7.11 | 0.68 | | | |
| | EC | | | | | | | | | | |
| IL-12 | | | | | | | | | | | |
| (p70) | | | | | | | | | | | |
| | Benign | 2.35 | 0.27 | <0.0001 | (1) vs. | <0.0001 | 2.27 | 0.29 | <0.0001 | (1) vs. | <0.0001 |
| | | | | | (3) | | | | | (3) | |
| | Hyperplasia | 2.47 | 0.66 | | (1) vs. | 0.001 | 3.08 | 0.71 | | (1) vs. | 0.0002 |
| | | | | | (4) | | | | | (4) | |
| | Low-grade | 3.47 | 0.38 | | (2) vs. | 0.02 | 4.13 | 0.46 | | (2) vs. | 0.02 |
| | EEC | | | | (3) | | | | | (3) | |
| | Other | 3.74 | 0.78 | | | | 4.26 | 0.80 | | | |
| | EC | | | | | | | | | | |
| IL-13 | | | | | | | | | | | |
| | Benign | 2.33 | 0.34 | <0.0001 | (1) vs. | 0.004 | 2.24 | 0.37 | <0.0001 | (1) vs. | 0.003 |
| | | | | | (2) | | | | | (2) | |
| | Hyperplasia | 3.35 | 0.84 | | (1) vs. | <0.0001 | 3.77 | 0.91 | | (1) vs. | <0.000 |
| | | | | | (3) | | | | | (3) | 1 |
| | Low-grade | 2.82 | 0.49 | | (1) vs | <0.0001 | 3.30 | 0.59 | | (1) vs. | 0.0002 |
| | EEC | | | | (4) | | | | | (4) | |
| | Other | 3.14 | 0.98 | | | | 3.49 | 1.02 | | | |
| | EC | | | | | | | | | | |
| IL-15 | | | | | | | | | | | |
| | Benign | 2.16 | 0.31 | <0.0001 | (1) vs. | <0.0001 | 1.77 | 0.34 | <0.0001 | (1) vs. | <0.0001 |
| | | | | | (3) | | | | | (3) | |
| | Hyperplasia | 1.81 | 0.77 | | (1) vs. | 0.0004 | 2.61 | 0.82 | | (1) vs. | <0.0001 |
| | | | | | (4) | | | | | (4) | |
| | Low-grade | 3.69 | 0.45 | | (2) vs. | 0.005 | 4.67 | 0.53 | | (2) vs. | 0.003 |
| | EEC | | | | (3) | | | | | (3) | |
| | Other | 2.85 | 0.90 | | | | 3.71 | 0.92 | | | |
| | EC | | | | | | | | | | |
| IL-17A | | | | | | | | | | | |
| | Benign | 1.97 | 0.34 | 0.09 | | | 2.34 | 0.37 | 0.05 | | |
| | Hyperplasia | 2.53 | 0.84 | | | | 2.46 | 0.91 | | | |
| | Low-grade | 3.13 | 0.49 | | | | 2.87 | 0.59 | | | |
| | EEC | | | | | | | | | | |
| | Other | 2.45 | 0.99 | | | | 1.97 | 1.02 | | | |
| | EC | | | | | | | | | | |
| IL-36γ | | | | | | | | | | | |
| | Benign | 322.65 | 76.56 | 0.44 | | | 310.29 | 83.78 | 0.59 | | |
| | Hyperplasia | 617.66 | 187.53 | | | | 536.07 | 204.47 | | | |
| | Low-grade | 428.30 | 109.29 | | | | 383.17 | 132.39 | | | |
| | EEC | | | | | | | | | | |
| | Other | 815.61 | 220.66 | | | | 801.95 | 229.54 | | | |
| | EC | | | | | | | | | | |
| IP-10 | | | | | | | | | | | |
| | Benign | 442.79 | 87.28 | <0.0001 | (1) vs. | 0.003 | 436.76 | 95.10 | <0.0001 | (1) vs. | 0.001 |
| | | | | | (2) | | | | | (2) | |
| | Hyperplasia | 1182.94 | 213.80 | | (1) vs. | <0.0001 | 1264.68 | 232.11 | | (1) vs. | <0.0001 |
| | | | | | (3) | | | | | (3) | |
| | Low-grade | 1212.83 | 124.60 | | (1) vs. | <0.0001 | 1294.76 | 150.28 | | (1) vs. | <0.0001 |
| | EEC | | | | (4) | | | | | (4) | |
| | Other | 2076.27 | 251.58 | | (2) vs. | 0.04 | 2148.59 | 260.57 | | | |
| | EC | | | | (4) | | | | | | |
| LAG-3 | | | | | | | | | | | |
| | Benign | 478.59 | 124.49 | <0.0001 | (1) vs. | 0.0004 | 638.88 | 134.12 | <0.0001 | (1) vs. | 0.001 |
| | | | | | (2) | | | | | (2) | |
| | Hyperplasia | 590.63 | 304.93 | | (1) vs. | <0.0001 | 630.10 | 327.34 | | (1) vs. | <0.0001 |
| | | | | | (3) | | | | | (3) | |
| | Low-grade | 746.64 | 177.70 | | (1) vs. | 0.004 | 701.88 | 211.94 | | (1) vs. | 0.008 |
| | EEC | | | | (4) | | | | | (4) | |
| | Other | 533.79 | 358.81 | | | | 362.52 | 367.47 | | | |
| | EC | | | | | | | | | | |
| Leptin | | | | | | | | | | | |
| | Benign | 286.62 | 41.35 | <0.0001 | (1) vs. | 0.04 | 333.02 | 44.24 | <0.0001 | (1) vs. | 0.001 |
| | | | | | (2) | | | | | (3) | |
| | Hyperplasia | 346.11 | 101.29 | | (1) vs. | <0.0001 | 218.04 | 107.97 | | (1) vs. | <0.0001 |
| | | | | | (3) | | | | | (4) | |
| | Low-grade | 493.89 | 59.03 | | (1) vs. | <0.0001 | 367.38 | 69.90 | | (2) vs. | 0.02 |
| | EEC | | | | (4) | | | | | (4) | |
| | Other | 771.20 | 119.18 | | (2) vs. | 0.01 | 657.40 | 121.20 | | | |
| | EC | | | | (4) | | | | | | |
| MCP-1 | | | | | | | | | | | |
| | Benign | 382.83 | 67.79 | <0.0001 | (1) vs. | <0.0001 | 366.43 | 74.07 | <0.0001 | (1) vs. | <0.0001 |
| | | | | | (2) | | | | | (2) | |
| | Hyperplasia | 1278.04 | 166.04 | | (1) vs. | <0.0001 | 1410.09 | 180.79 | | (1) vs. | <0.0001 |
| | | | | | (3) | | | | | (3) | |
| | Low-grade | 1453.56 | 96.77 | | (1) vs. | <0.0001 | 1574.90 | 117.05 | | (1) vs. | <0.0001 |
| | EEC | | | | (4) | | | | | (4) | |
| | Other | 2137.08 | 195.38 | | (2) vs. | 0.03 | 2217.97 | 202.95 | | (2) vs. | 0.05 |
| | EC | | | | (4) | | | | | (4) | |
| MCP-3 | | | | | | | | | | | |
| | Benign | 13.68 | 2.62 | <0.0001 | (1) vs. | <0.0001 | 12.71 | 2.84 | <0.0001 | (1) vs. | 0.01 |
| | | | | | (3) | | | | | (2) | |
| | Hyperplasia | 19.17 | 6.41 | | (1) vs. | <0.0001 | 26.06 | 6.94 | | (1) vs. | <0.0001 |
| | | | | | (4) | | | | | (3) | |
| | Low-grade | 28.13 | 3.74 | | (2) vs. | <0.0001 | 34.07 | 4.49 | | (1) vs. | <0.0001 |
| | EEC | | | | (3) | | | | | (4) | |
| | Other | 80.25 | 7.54 | | (2) vs. | <0.0001 | 84.44 | 7.79 | | (2) vs. | <0.0001 |
| | EC | | | | (4) | | | | | (4) | |
| | | | | | | | | | | (3) vs. | <0.0001 |
| | | | | | | | | | | (4) | |
| MDC | | | | | | | | | | | |
| | Benign | 92.77 | 6.53 | 0.005 | (1) vs. | 0.008 | 94.08 | 7.14 | 0.32 | | |
| | | | | | (3) | | | | | | |
| | Hyperplasia | 87.15 | 16.00 | | | | 95.65 | 17.43 | | | |
| | Low-grade | 121.35 | 9.32 | | | | 128.90 | 11.29 | | | |
| | EEC | | | | | | | | | | |
| | Other | 92.98 | 18.83 | | | | 96.60 | 19.57 | | | |
| | EC | | | | | | | | | | |
| MIF | | | | | | | | | | | |
| | Benign | 639.20 | 77.64 | <0.0001 | (1) vs. | 0.01 | 618.19 | 83.80 | 0.07 | | |
| | | | | | (2) | | | | | | |
| | Hyperplasia | 707.47 | 190.19 | | (1) vs. | <0.0001 | 664.75 | 204.54 | | | |
| | | | | | (3) | | | | | | |
| | Low-grade | 701.27 | 110.84 | | (1) vs. | 0.01 | 633.27 | 132.43 | | | |
| | EEC | | | | (4) | | | | | | |
| | Other | 774.23 | 223.80 | | | | 718.30 | 229.61 | | | |
| | EC | | | | | | | | | | |
| MIP-1α | | | | | | | | | | | |
| | Benign | 31.51 | 8.42 | <0.0001 | (1) vs. | 0.002 | 22.23 | 9.09 | <0.0001 | (1) vs. | 0.0004 |
| | | | | | (2) | | | | | (2) | |
| | Hyperplasia | 61.00 | 20.63 | | (1) vs. | <0.0001 | 81.39 | 22.18 | | (1) vs. | <0.0001 |
| | | | | | (3) | | | | | (3) | |
| | Low-grade | 55.54 | 12.02 | | (1) vs. | <0.0001 | 78.99 | 14.36 | | (2) vs. | <0.0001 |
| | EEC | | | | (4) | | | | | (3) | |
| | Other | 51.84 | 24.27 | | | | 71.58 | 24.90 | | | |
| | EC | | | | | | | | | | |
| MIP-1β | | | | | | | | | | | |
| | Benign | 46.73 | 11.75 | <0.0001 | (1) vs. | 0.004 | 51.27 | 12.80 | <0.0001 | (1) vs. | 0.003 |
| | | | | | (2) | | | | | (2) | |
| | Hyperplasia | 107.42 | 28.79 | | (1) vs. | <0.0001 | 129.15 | 31.24 | | (1) vs. | <0.0001 |
| | | | | | (3) | | | | | (3) | |
| | Low-grade | 136.23 | 16.78 | | (1) vs. | <0.0001 | 153.93 | 20.22 | | (1) vs. | <0.0001 |
| | EEC | | | | (4) | | | | | (4) | |
| | Other | 117.96 | 33.88 | | (2) vs. | 0.02 | 124.52 | 35.07 | | | |
| | EC | | | | (3) | | | | | | |
| OPN | | | | | | | | | | | |
| | Benign | 381.82 | 51.39 | <0.0001 | (1) vs. | 0.01 | 424.30 | 56.06 | <0.0001 | (1) vs. | <0.0001 |
| | | | | | (2) | | | | | (4) | |
| | Hyperplasia | 706.20 | 125.89 | | (1) vs. | 0.01 | 689.29 | 136.83 | | (3) vs. | 0.01 |
| | | | | | (3) | | | | | (4) | |
| | Low-grade | 577.96 | 73.36 | | (1) vs. | <0.0001 | 527.05 | 88.59 | | | |
| | EEC | | | | (4) | | | | | | |
| | Other | 1148.28 | 148.13 | | (3) vs. | 0.003 | 1075.58 | 153.61 | | | |
| | EC | | | | (4) | | | | | | |
| PD-1 | | | | | | | | | | | |
| | Benign | 10.93 | 2.45 | 0.5 | | | 10.62 | 2.68 | 0.45 | | |
| | Hyperplasia | 20.25 | 6.00 | | | | 17.78 | 6.54 | | | |
| | Low-grade | 18.09 | 3.50 | | | | 16.05 | 4.24 | | | |
| | EEC | | | | | | | | | | |
| | Other | 6.11 | 7.06 | | | | 4.97 | 7.35 | | | |
| | EC | | | | | | | | | | |
| PD-L1 | | | | | | | | | | | |
| | Benign | 1.73 | 0.34 | 0.001 | (1) vs. | 0.006 | 1.54 | 0.37 | 0.001 | (1) vs. | 0.005 |
| | | | | | (2) | | | | | (2) | |
| | Hyperplasia | 2.98 | 0.84 | | | | 3.30 | 0.91 | | | |
| | Low-grade | 1.73 | 0.49 | | | | 2.23 | 0.59 | | | |
| | EEC | | | | | | | | | | |
| | Other | 1.00 | 0.99 | | | | 1.43 | 1.02 | | | |
| | EC | | | | | | | | | | |
| PD-L2 | | | | | | | | | | | |
| | Benign | 85.89 | 9.57 | <0.0001 | (1) vs. | <0.0001 | 89.31 | 10.36 | <0.0001 | (1) vs. | <0.0001 |
| | | | | | (3) | | | | | (3) | |
| | Hyperplasia | 66.61 | 23.44 | | (1) vs. | <0.0001 | 80.90 | 25.28 | | (1) vs. | <0.0001 |
| | | | | | (4) | | | | | (4) | |
| | Low-grade | 130.70 | 13.66 | | (2) vs. | 0.02 | 134.49 | 16.37 | | (2) vs. | 0.04 |
| | EEC | | | | (3) | | | | | (3) | |
| | Other | 247.94 | 27.58 | | (2) vs. | <0.0001 | 243.38 | 28.37 | | (2) vs. | <0.0001 |
| | EC | | | | (4) | | | | | (4) | |
| | | | | | (3) vs. | 0.0001 | | | | (3) vs. | 0.0004 |
| | | | | | (4) | | | | | (4) | |
| PDGF-AA | | | | | | | | | | | |
| | Benign | 40.39 | 7.44 | <0.0001 | (1) vs. | <0.0001 | 38.49 | 7.97 | 0.0001 | (1) vs. | <0.0001 |
| | | | | | (3) | | | | | (3) | |
| | Hyperplasia | 15.56 | 18.23 | | (1) vs. | <0.0001 | 34.91 | 19.45 | | (1) vs. | <0.0001 |
| | | | | | (4) | | | | | (4) | |
| | Low-grade | 82.78 | 10.62 | | (2) vs. | 0.003 | 100.70 | 12.59 | | (2) vs. | 0.002 |
| | EEC | | | | (3) | | | | | (3) | |
| | Other | 92.05 | 21.45 | | (2) vs. | <0.0001 | 101.44 | 21.83 | | (2) vs. | 0.0003 |
| | EC | | | | (4) | | | | | (4) | |
| PDGF-AB/BB | | | | | | | | | | | |
| | Benign | 134.51 | 12.33 | 0.0002 | (1) vs. | 0.0003 | 127.84 | 13.40 | <0.0001 | (1) vs. | <0.0001 |
| | | | | | (3) | | | | | (3) | |
| | Hyperplasia | 82.22 | 30.20 | | (2) vs. | 0.02 | 110.83 | 32.71 | | (2) vs. | 0.03 |
| | | | | | (3) | | | | | (3) | |
| | Low-grade | 181.67 | 17.60 | | | | 208.54 | 21.18 | | | |
| | EEC | | | | | | | | | | |
| | Other | 150.43 | 35.54 | | | | 168.79 | 36.72 | | | |
| | EC | | | | | | | | | | |
| Prolactin | | | | | | | | | | | |
| | Benign | 402.27 | 128.99 | <0.0001 | (1) vs. | <0.0001 | 349.39 | 140.93 | <0.0001 | (1) vs. | <0.0001 |
| | | | | | (3) | | | | | (3) | |
| | Hyperplasia | 651.42 | 315.97 | | (1) vs. | 0.0002 | 713.63 | 343.95 | | (1) vs. | 0.002 |
| | | | | | (4) | | | | | (4) | |
| | Low-grade | 1001.01 | 184.14 | | | | 1038.70 | 222.70 | | | |
| | EEC | | | | | | | | | | |
| | Other | 265.50 | 371.80 | | | | 306.56 | 386.12 | | | |
| | EC | | | | | | | | | | |
| PSA (total) | | | | | | | | | | | |
| | Benign | 894.57 | 127.11 | 0.04 | (2) vs. | 0.04 | 788.37 | 136.54 | 0.01 | (2) vs. | 0.04 |
| | | | | | (4) | | | | | (4) | |
| | Hyperplasia | 722.69 | 311.36 | | | | 1059.13 | 333.26 | | (3) vs. | 0.03 |
| | | | | | | | | | | (4) | |
| | Low-grade | 288.03 | 181.45 | | | | 597.33 | 215.77 | | | |
| | EEC | | | | | | | | | | |
| | Other | 6.53 | 366.38 | | | | 220.36 | 374.12 | | | |
| | EC | | | | | | | | | | |
| RANTES | | | | | | | | | | | |
| | Benign | 213.40 | 35.20 | <0.0001 | (1) vs. | <0.0001 | 179.84 | 38.03 | <0.0001 | (1) vs. | <0.0001 |
| | | | | | (3) | | | | | (3) | |
| | Hyperplasia | 181.15 | 86.22 | | (1) vs. | <0.0001 | 240.90 | 92.82 | | (1) vs. | 0.001 |
| | | | | | (4) | | | | | (4) | |
| | Low-grade | 334.63 | 50.25 | | (2) vs. | 0.02 | 398.70 | 60.10 | | | |
| | EEC | | | | (3) | | | | | | |
| | Other | 267.96 | 101.46 | | | | 321.26 | 104.20 | | | |
| | EC | | | | | | | | | | |
| SCF | | | | | | | | | | | |
| | Benign | 5.01 | 1.11 | <0.0001 | (1) vs. | 0.0001 | 5.26 | 1.21 | <0.0001 | (1) vs. | 0.0003 |
| | | | | | (2) | | | | | (2) | |
| | Hyperplasia | 16.37 | 2.72 | | (1) vs. | <0.0001 | 17.48 | 2.94 | | (1) vs. | <0.0001 |
| | | | | | (3) | | | | | (3) | |
| | Low-grade | 14.06 | 1.59 | | (1) vs. | <0.0001 | 15.35 | 1.91 | | (1) vs. | <0.0001 |
| | EEC | | | | (4) | | | | | (4) | |
| | Other | 13.96 | 3.21 | | | | 14.51 | 3.30 | | | |
| | EC | | | | | | | | | | |
| sCD40L | | | | | | | | | | | |
| | Benign | 24.68 | 4.34 | 0.0001 | (1) vs. | 0.001 | 24.28 | 4.67 | <0.0001 | (1) vs. | <0.0001 |
| | | | | | (3) | | | | | (3) | |
| | Hyperplasia | 10.26 | 10.64 | | (2) vs. | 0.04 | 15.19 | 11.39 | | (1) vs. | 0.008 |
| | | | | | (3) | | | | | (4) | |
| | Low-grade | 14.34 | 6.20 | | | | 20.39 | 7.37 | | (2) vs. | 0.02 |
| | EEC | | | | | | | | | (3) | |
| | Other | 27.41 | 12.52 | | | | 30.45 | 12.78 | | | |
| | EC | | | | | | | | | | |
| sFas | | | | | | | | | | | |
| | Benign | 123.82 | 12.97 | <0.0001 | (1) vs. | <0.0001 | 125.39 | 13.99 | <0.0001 | (1) vs. | <0.0001 |
| | | | | | (3) | | | | | (3) | |
| | Hyperplasia | 122.92 | 31.77 | | (1) vs. | 0.01 | 164.92 | 34.15 | | (1) vs. | 0.01 |
| | | | | | (4) | | | | | (4) | |
| | Low-grade | 277.84 | 18.52 | | (2) vs. | 0.004 | 307.35 | 22.11 | | (2) vs. | 0.02 |
| | EEC | | | | (3) | | | | | (3) | |
| | Other | 215.16 | 37.39 | | | | 226.44 | 38.33 | | | |
| | EC | | | | | | | | | | |
| sFasL | | | | | | | | | | | |
| | Benign | 7.04 | 1.53 | 0.001 | (1) vs. | 0.001 | 5.94 | 1.67 | 0.001 | (1) vs. | 0.0004 |
| | | | | | (3) | | | | | (3) | |
| | Hyperplasia | 5.84 | 3.75 | | | | 8.41 | 4.07 | | | |
| | Low-grade | 6.27 | 2.19 | | | | 9.18 | 2.64 | | | |
| | EEC | | | | | | | | | | |
| | Other | 4.15 | 4.41 | | | | 6.58 | 4.57 | | | |
| | EC | | | | | | | | | | |
| TGF-α | | | | | | | | | | | |
| | Benign | 8.33 | 1.01 | <0.0001 | (1) vs. | 0.02 | 10.07 | 1.07 | <0.0001 | (1) vs. | <0.0001 |
| | | | | | (2) | | | | | (3) | |
| | Hyperplasia | 14.12 | 2.48 | | (1) vs. | <0.0001 | 15.55 | 2.62 | | (1) vs. | <0.0001 |
| | | | | | (3) | | | | | (4) | |
| | Low-grade | 27.95 | 1.45 | | (1) vs. | <0.0001 | 27.42 | 1.70 | | (2) vs. | 0.003 |
| | EEC | | | | (4) | | | | | (3) | |
| | Other | 26.70 | 2.92 | | (2) vs. | 0.0003 | 24.52 | 2.94 | | | |
| | EC | | | | (3) | | | | | | |
| | | | | | (2) vs. | 0.01 | | | | | |
| | | | | | (4) | | | | | | |
| TIM-3 | | | | | | | | | | | |
| | Benign | 26.56 | 4.31 | <0.0001 | (1) vs. | <0.0001 | 29.29 | 4.65 | <0.0001 | (1) vs. | 0.006 |
| | | | | | (2) | | | | | (2) | |
| | Hyperplasia | 57.93 | 10.55 | | (1) vs. | <0.0001 | 59.52 | 11.34 | | (1) vs. | <0.0001 |
| | | | | | (3) | | | | | (3) | |
| | Low-grade | 91.95 | 6.15 | | (1) vs. | <0.0001 | 88.48 | 7.34 | | (1) vs. | <0.0001 |
| | EEC | | | | (4) | | | | | (4) | |
| | Other | 114.78 | 12.41 | | | | 108.65 | 12.73 | | | |
| | EC | | | | | | | | | | |
| TLR2 | | | | | | | | | | | |
| | Benign | 176.33 | 16.52 | <0.0001 | (1) vs. | <0.0001 | 164.42 | 18.05 | <0.0001 | (1) vs. | <0.0001 |
| | | | | | (3) | | | | | (3) | |
| | Hyperplasia | 228.12 | 40.47 | | | | 243.54 | 44.06 | | | |
| | Low-grade | 315.57 | 23.59 | | | | 338.91 | 28.53 | | | |
| | EEC | | | | | | | | | | |
| | Other | 284.07 | 47.62 | | | | 308.43 | 49.46 | | | |
| | EC | | | | | | | | | | |
| TNFα | | | | | | | | | | | |
| | Benign | 15.01 | 3.80 | <0.0001 | (1) vs. | 0.008 | 8.65 | 4.07 | <0.0001 | (1) vs. | 0.001 |
| | | | | | (2) | | | | | (2) | |
| | Hyperplasia | 16.88 | 9.31 | | (1) vs. | <0.0001 | 24.73 | 9.94 | | (1) vs. | <0.0001 |
| | | | | | (3) | | | | | (3) | |
| | Low-grade | 30.08 | 5.43 | | (1) vs. | <0.0001 | 40.75 | 6.43 | | (1) vs. | <0.0001 |
| | EEC | | | | (4) | | | | | (4) | |
| | Other | 29.66 | 10.96 | | (2) vs. | 0.03 | 40.45 | 11.16 | | (2) vs. | 0.04 |
| | EC | | | | (3) | | | | | (3) | |
| | | | | | (2) vs. | 0.01 | | | | (2) vs. | 0.03 |
| | | | | | (4) | | | | | (4) | |
| TNFβ | | | | | | | | | | | |
| | Benign | 8.96 | 2.30 | 0.002 | (1) vs. | 0.002 | 7.96 | 2.52 | 0.02 | (1) vs. | 0.02 |
| | | | | | (3) | | | | | (3) | |
| | Hyperplasia | 8.98 | 5.65 | | | | 11.13 | 6.14 | | | |
| | Low-grade | 6.35 | 3.29 | | | | 9.04 | 3.98 | | | |
| | EEC | | | | | | | | | | |
| | Other | 10.23 | 6.64 | | | | 12.36 | 6.90 | | | |
| | EC | | | | | | | | | | |
| TRAIL | | | | | | | | | | | |
| | Benign | 29.39 | 5.18 | <0.0001 | (1) vs. | 0.02 | 37.50 | 5.53 | <0.0001 | (1) vs. | <0.0001 |
| | | | | | (2) | | | | | (3) | |
| | Hyperplasia | 61.51 | 12.68 | | (1) vs. | <0.0001 | 66.44 | 13.50 | | (1) vs. | <0.0001 |
| | | | | | (3) | | | | | (4) | |
| | Low-grade | 105.73 | 7.39 | | (1) vs. | <0.0001 | 103.98 | 8.74 | | (2) vs. | 0.002 |
| | EEC | | | | (4) | | | | | (3) | |
| | Other | 141.69 | 14.92 | | (2) vs. | 0.0004 | 132.52 | 15.16 | | (2) vs. | 0.003 |
| | EC | | | | (3) | | | | | (4) | |
| | | | | | (2) vs. | 0.0001 | | | | | |
| | | | | | (4) | | | | | | |
| VEGF | | | | | | | | | | | |
| | Benign | 69.60 | 48.59 | <0.0001 | (1) vs. | <0.0001 | 125.15 | 52.29 | <0.0001 | (1) vs. | <0.0001 |
| | | | | | (3) | | | | | (3) | |
| | Hyperplasia | 75.70 | 119.02 | | (1) vs. | <0.0001 | 146.56 | 127.62 | | (1) vs. | <0.0001 |
| | | | | | (4) | | | | | (4) | |
| | Low-grade | 517.47 | 69.36 | | (2) vs. | <0.0001 | 514.77 | 82.63 | | (2) vs. | 0.0002 |
| | EEC | | | | (3) | | | | | (3) | |
| | Other | 1129.78 | 140.05 | | (2) vs. | <0.0001 | 1073.35 | 143.26 | | (2) vs. | <0.0001 |
| | EC | | | | (4) | | | | | (4) | |
| | | | | | (3) vs. | 0.04 | | | | | |
| | | | | | (4) | | | | | | |

(1) benign;
(2) hyperplasia;
(3) low-grade EEC;
(4) other EC.

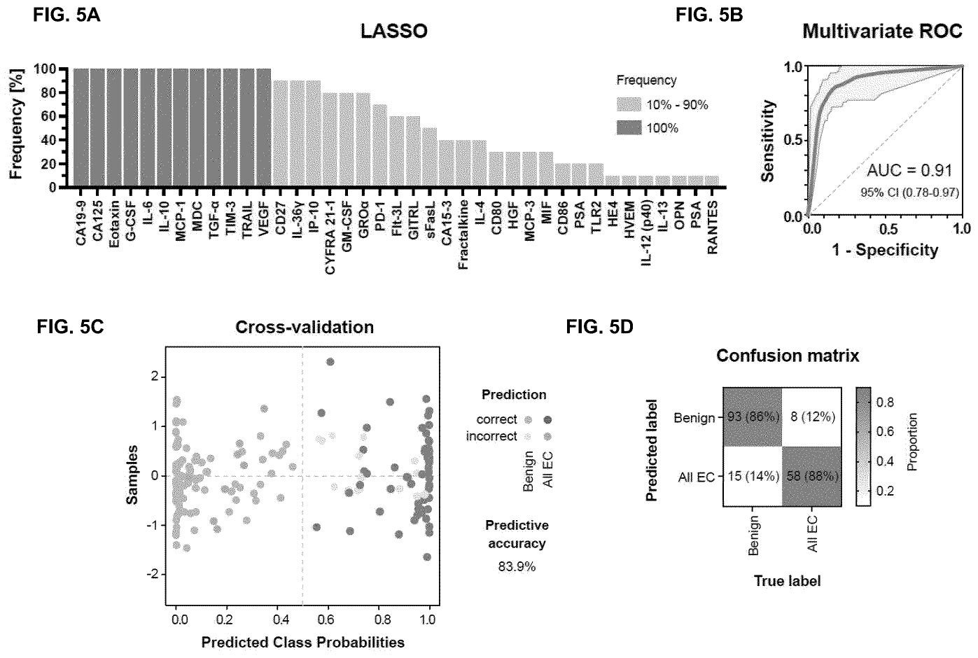

Machine-learning modeling to predict EC. To evaluate the ability of multiple cervicovaginal protein biomarkers to predict the disease group (all EC subtypes vs. benign conditions), the logistic regression classification with the Monte Carlo cross-validation was used (FIG. 5A-5D). The predictive model was built using 12 protein biomarkers with 100% frequency in the least absolute shrinkage and selection operator method (i.e., CA19-9, CA125, eotaxin, G-CSF, IL-6, IL-10, MCP-1, MDC, TGF-α, TIM-3, TRAIL, and VEGF) (FIG. 5A). Five out of 12 biomarkers (IL-10, TGF-α, TIM-3, TRAIL, and VEGF) exhibited good discriminatory properties (AUC>0.8) for both EC subtype groups when compared to benign conditions, and three biomarkers (IL-6, MCP-1, and MDC) exhibited good discriminatory properties for other EC subtype, but not for the low-grade EEC group, in the previous univariate ROC analysis (FIG. 3A-3G). In a subsequent multivariate ROC analysis, the model based on the selected 12 biomarkers demonstrated an excellent ability to discriminate between patients with EC and benign conditions (average AUC 0.91) (FIG. 5B). Overall, the average predictive accuracy of the model based on 100 cross-validations was 83.9% (FIG. 5C). A confusion matrix was also created to show the proportion of time each sample obtained correct classification (FIG. 5D). Ninety-three out of 108 benign samples were correctly classified (sensitivity of 86.1%), and 58 out of 66 EC samples were correctly classified using the model (specificity of 87.9%). Overall, this analysis showed that coupling multiple cervicovaginal biomarkers with machine learning algorithms can increase the ability of created models to accurately predict the disease group.

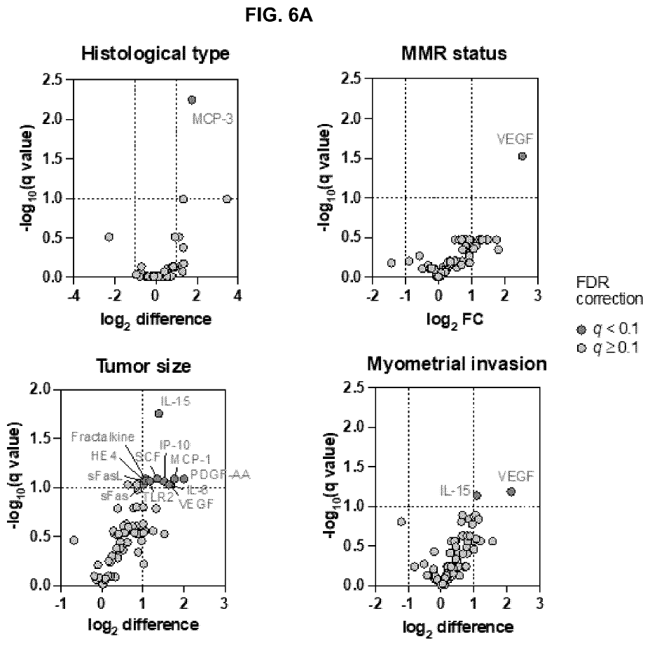

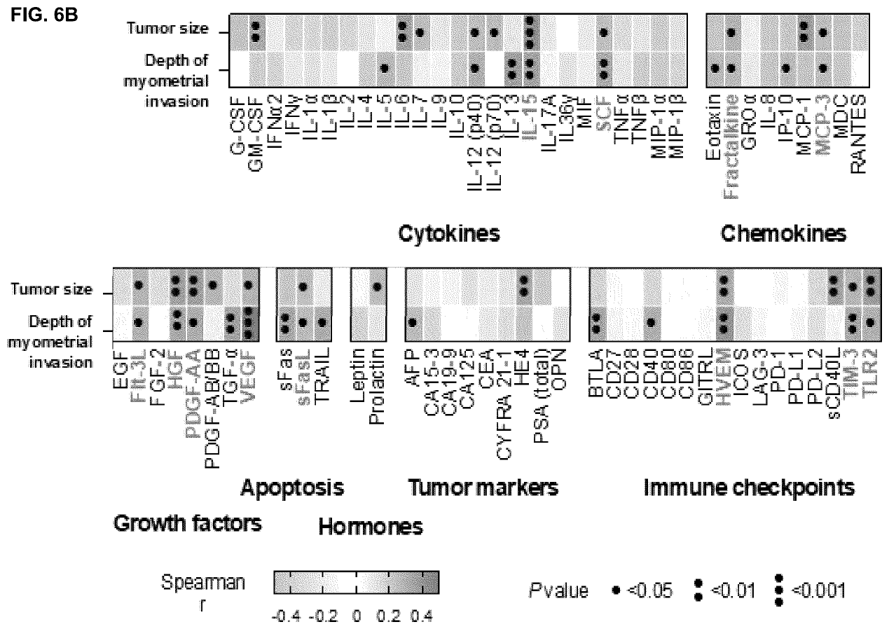

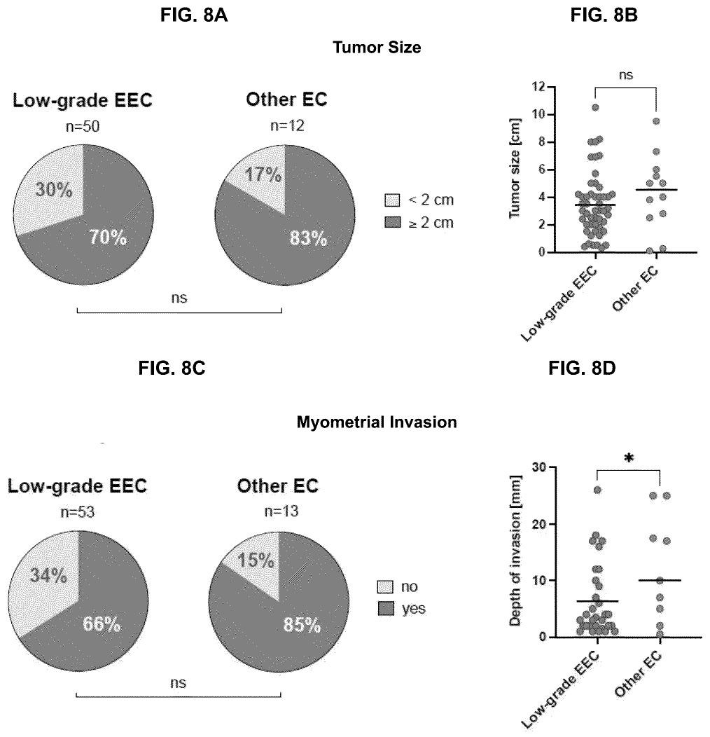

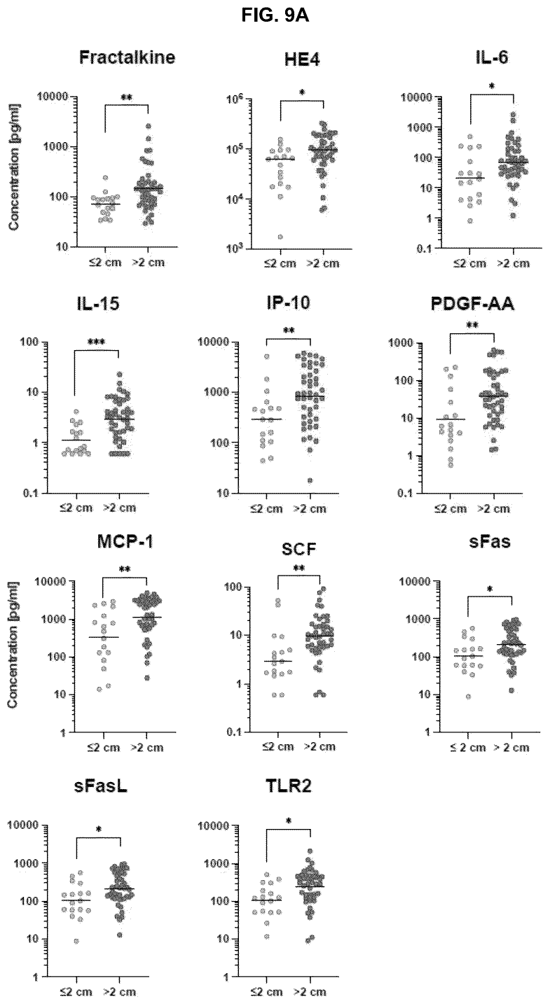

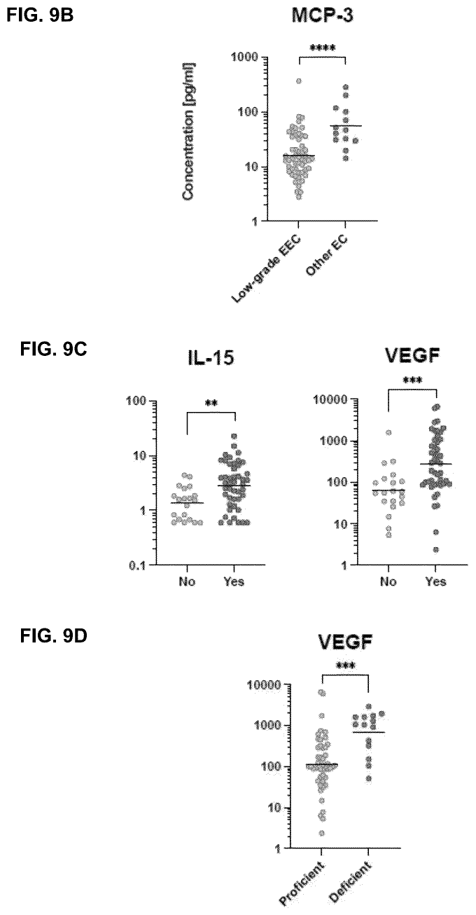

Cervicovaginal proteins and the severity of EC. To identify relationships between the cervicovaginal biomarker levels and the severity of EC, data was extracted from pathology reports on FIGO stage, histological type, and grade, tumor size, presence and depth of myometrial invasion, presence of lymphovascular invasion, and mismatch repair (MMR) protein expression (Table 3 and FIGS. 9A, 9B, 9C, and 9D). Out of 66 women diagnosed with EC, 59 (89.4%) had endometrioid carcinomas or adenocarcinomas. The majority of EC (87.1%) were stage I tumors, were of low grade (i.e., grade 1 or 2; 86.4%), and had size greater than 2 cm (70%) (FIGS. 8A, 8B, and 8C). Myometrial and lymphovascular invasion were present in 69.7% and 6.2% of EC tumors, respectively. The MMR deficiency (i.e., loss of MLH1, PMS2, MSH2, or MSH6 expression) was observed in 23.3% of EC tumors. EC tumors were categorized based on histological type (low-grade EEC vs. other EC subtypes), MMR status (MMR-deficient vs. MMR-proficient), size (≤2 cm vs. >2 cm), and presence of myometrial invasion and compared the cervicovaginal levels of protein biomarkers between these subgroups (FIG. 6A and FIGS. 9A, 9B, 9C, and 9D). The FIGO stage or lymphovascular invasion were not analyzed due to the unbalanced distribution of these characteristics among our cohort (Table 3). Following the false discovery rate correction for multiple comparisons (q<0.1), the analysis revealed that only one protein, MCP-3, was significantly elevated in other EC subtypes compared to low-grade EEC. When tumors were stratified based on MMR status, VEGF was significantly elevated in CVL samples from patients with MMR-deficient EC. Furthermore, cervicovaginal levels of 12 proteins (fractalkine, HE4, IL-6, IL-15, IP-10, MCP-1, PDGF-AA, sFas, sFasL, SCF, TLR2, VEGF) were significantly elevated in patients with larger tumors (>2 cm) compared to patients with smaller tumors (≤2 cm). IL-15 and VEGF also levels varied between groups stratified based on the presence of myometrial invasion. In addition, a correlation analysis was performed between levels of proteins in CVL and size of tumors (measured in cm), and depth of myometrial invasion (measured in mm) (FIG. 6B). Twelve protein markers (cytokines: IL-15 and SCF; chemokines: fractalkine and MCP-3; growth factors: FIt-3L, HGF, PDGF-AA, and VEGF; an apoptosis-related protein, sFasL; and immune checkpoint proteins: HVEM, TIM-3, and TLR2) significantly correlated with both tumor size and depth of myometrial invasion. Notably, four biomarkers identified in the correlation analysis (TIM-3, VEGF, TGF-α, and TRAIL) were highly discriminatory for both low-grade EEC and other EC subtypes (FIG. 3A-3G). Among them, TIM-3 and VEGF are associated with tumor size, myometrial invasion, and MMR status, whereas TGF-α and TRAIL levels are associated with myometrial invasion, but not other tumor characteristics. Overall, this analysis revealed that cervicovaginal sampling of protein biomarkers can allow for detection of EC detection and stratification of patients based on tumor characteristics

TABLE 3
Characteristics of EC tumors in our cohort. Data on histological type, FIGO
stage, tumor grade, tumor size, presence and depth of myometrial invasion,
presence ofl ymphovascular invasion, and MMR protein status were
extracted from pathology reports. n indicates data availability.

| Characteristics | | n (%) |
|---|---|---|
| Histological type (n = 66) | Endometrioid adenocarcinoma | 33 (50.0) |
| | Endometrioid carcinoma | 26 (39.4) |
| | Serous carcinoma | 4 (6.1) |
| | Other | 3 (4.5) |
| FIGO stage (n = 62) | I | 1 (1.6) |
| | IA | 43 (69.4) |
| | IB | 10 (16.1) |
| | II | 2 (3.2) |
| | IIIC | 4 (6.5) |
| | IV | 2 (3.2) |
| Size (n = 62) | ≤2 cm | 17 (27.4 |
| | >2 cm | 45 (72.6) |
| Myometrial invasion (n = 66) | no | 20 (30.3) |
| | yes | 46 (69.7) |
| | depth reported | 40 (60.6) |
| | depth not reported | 6 (9.1) |
| Lymphovascular invasion | no | 61 (93.8) |
| (n = 65) | yes | 4 (6.2) |
| MMR protein status (n = 60) | MMR-proficient | 46 (76.6) |
| | MMR-deficient | 14 (23.3) |
| loss of nuclear expression | MLH1 | 9 (15.0) |
| | PMS2 | 11 (18.3) |
| | MSH2 | 1 (1.7) |
| | MSH6 | 4 (6.7) |

### Example 2

The following is a non-limiting example of the present invention. It is to be understood that said example is not intended to limit the present invention in any way. Equivalents or substitutes are within the scope of the present invention.

Abbreviations. Endometrial cancer—EC, Cervicovaginal lavage—CVL, Endometrial endometrioid cancer—EEC, Body mass index—BMI, Mismatch repair—MMR, Transvaginal ultrasound—TVUS, Area under curve—AUC, Receiver operating characteristics—ROC.

Patient enrollment. A total of 192 cis-gender women undergoing a hysterectomy for benign or malignant conditions were enrolled at three clinical sites across Phoenix, Arizona, USA: Banner University Medical Center-Phoenix, Dignity Health Chandler Regional Medical Center and Valleywise Health Medical Center. The patients were recruited from June 2018 to February 2020. Based on the histopathological results of hysterectomy samples, patients were categorized into one of the following groups: benign conditions (n=108), endometrial hyperplasia (n=18), or EC (n=66). The EC group was further stratified into grade 1 or 2 endometrial endometrioid carcinoma (EEC) (n=53) and other EC (n=13), which included grade 3 EEC and other non-endometrioid histopathological subtypes. The inclusion and exclusion criteria were previously described in Example 1 above. The exclusion criteria were confirmed through physician's pelvic exam and medical records and/or self-reported.

Sample Collection. Prior to a hysterectomy procedure, CVL samples were collected by a surgeon, before vaginal sterilization, and after anesthesia. CVL samples were obtained with a non-lubricated speculum, and 10 ml of sterile 0.9% saline solution (Teknova, Hollister, CA). Samples were placed on ice and frozen at −80° C. within an hour of collection. Before downstream analyses, samples were processed as follows; samples were thawed on ice, centrifuged (700×g at 4° C. for 10 minutes), aliquoted to limit multiple freeze-thaw cycles, and stored at −80° C., as described above in Example 1.

Soluble Protein Quantification. A total of 19 proteins were quantified in CVL samples using the Milliplex MAP Magnetic Bead Immunoassays: Human Circulating Cancer Biomarker Panel 3 and Human Angiogenesis/Growth Factor Panel 1 (Millipore, Billerica, MA) targeting the following proteins: angiopoietin-2, bone morphogenetic protein (BMP)-9, cathepsin-D, endoglin, endothelin-1, ferritin, fibroblast activation protein (FAP), fibroblast growth factor (FGF)-1, follistatin, galectin-3, heparin-binding epidermal growth factor (HB-EGF), insulin-like growth factor-binding protein (IGFBP)-3, melanoma inhibitory activity (MIA), myeloperoxidase (MPO), placental growth factor (PLGF), sex hormone binding globulin (SHBG), vascular endothelial growth factor (VEGF)-A, VEGF-C and VEGF-D. The Bio-Plex 200 instrument was used to collect data, and the Manager 5.0 software was utilized to analyze the data (Bio-Rad, Hercules, CA). To determine the protein concentrations, a five-parametric logistic regression curve fit was used. Each sample was analyzed in duplicate. Any concentration values below the minimum detectable concentration were replaced with half of the minimum detection limit as indicated in the manufacturer's guidelines. Prior to data analysis, protein concentrations were log₁₀-transformed before data analysis.

Hierarchical clustering analysis. Unsupervised hierarchical clustering analysis was conducted to visualize the relationships between relative levels of protein biomarkers and patient characteristics such as body mass index (BMI), disease group, and menopausal status. Prior to analysis, protein concentrations were mean-centered and variance-scaled. The Euclidean distance measure and the Ward linkage method were applied for sample clustering. ClustVis web tool was utilized for hierarchical clustering analysis.

Fold Change Analysis. A fold change analysis was used to compare the absolute value of change of the means of each protein between the two disease groups. Multiple t-tests with false discovery rate (FDR) correction (q<0.05) were utilized to determine significant differences in protein biomarker levels between disease groups. A heatmap was then used to visualize the data from fold change and two-sample t-test analyses. Proteins were significantly upregulated or downregulated based on a fold change (FC≥2 or FC≤−2) and q value (q<0.05).

Univariate and multivariate receiver operating characteristics (ROC) analyses. A univariate ROC analysis was performed using Prism 9.0 (GraphPad Software, Boston, MA, USA) on each protein to identify biomarkers discriminating disease groups with high sensitivity and specificity. A ROC curve was generated, and the area under the curve (AUC) was calculated. Any proteins with an AUC of greater than or equal to 0.8 were considered good discriminatory biomarkers. Multivariate ROC was conducted using MetaboAnalyst 6.0. Protein biomarkers were selected based on high least absolute shrinkage and selection operator (LASSO) frequencies and empirical testing of combinations of proteins. A Monte Carlo cross-validation model was utilized to assess the performance of the multivariate model, using ⅔ of the samples for training and ⅓ of the samples for testing. A ROC curve was generated from the averaged results of 100 cross-validations. The confusion matrix and the AUC of the ROC curves were calculated with a probability threshold of 0.5. Cohen's kappa was utilized to assess the inter-rater reliability of the multivariate biomarker model by calculating the level of agreement between the predictive label and the actual label of patients. A Cohen's kappa of above or equal to 0.7 is considered a substantial agreement.

Other Statistical Analysis. To assess differences in patient characteristics and demographics, a Kruskal-Wallis test was employed for continuous variables, while Fisher's exact test was applied to categorical variables. Statistical differences in the protein concentrations between disease groups were assessed using a one-way analysis of variance (ANOVA). If the difference was significant, multiple pairwise comparisons were performed with Bonferroni's correction. Comparisons were adjusted for age and BMI.

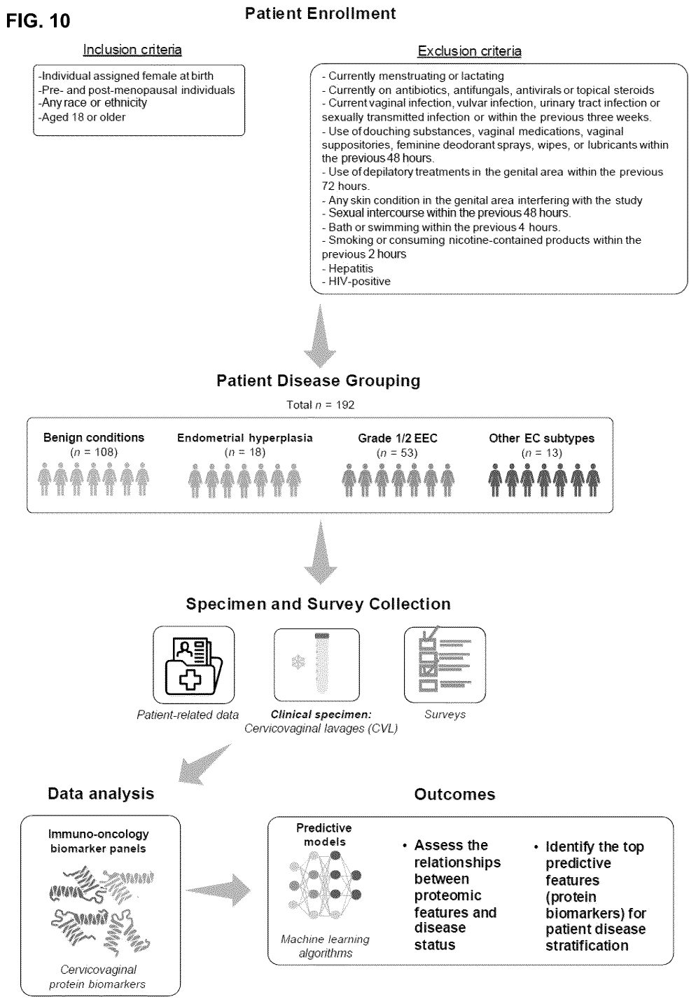

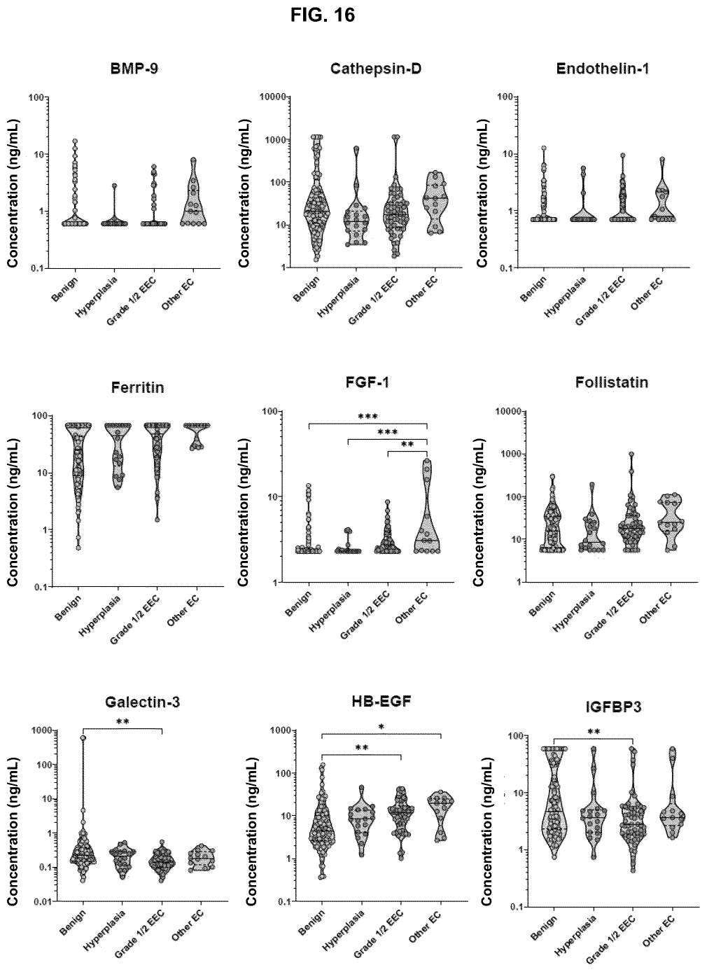

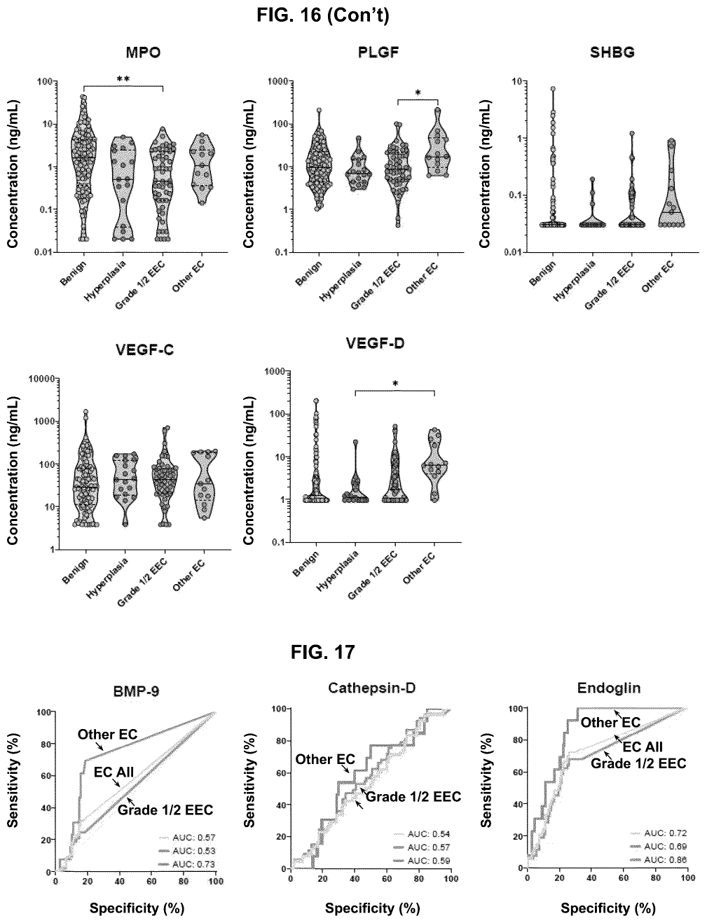

Patient demographics and characteristics. Patient inclusion and exclusion criteria are outlined in FIG. 10. Patient characteristics and demographics are summarized in Table 1 and detailed patient characteristics and demographics can be found in FIG. 16. This cohort was described in Example 1. Briefly, patients undergoing hysterectomy were stratified by disease groups: benign conditions (n=108), endometrial hyperplasia (n=18), grade 1/2 EEC (n=53) and other EC types (n=13) based on histopathology of biopsy samples. The average age of the cohort was 51 years old; the average BMI was 34.8 kg/m². Most patients were Caucasian (74.7%), and the study included a relatively high percentage (26.2%) of patients identifying as Hispanic. Overall statistical (p<0.0001) difference in the mean age of women was observed between disease groups. Specifically, patients with grade 1/2 EEC (58.73 years) and other EC types (60.77 years) were older than patients with benign conditions (45.55 years) (p<0.0001). Endometrial hyperplasia patients (54.11 years) were also statistically (p<0.01) older than patients with benign conditions. Furthermore, menopausal status significantly (p<0.0001) differed between the disease groups. Of grade 1/2 EEC patients, 76.47% were postmenopausal, which was significantly (p<0.0001) higher than the 43.16% of benign patients who were postmenopausal. When comparing the BMI of patients between the disease groups, endometrial hyperplasia patients (41.49 kg/m²) and grade 1/2 EEC patients (40.29 kg/m²) had significantly (p<0.0001) higher BMI than benign patients (30.63 kg/m²). Additionally, wendometrial hyperplasia patients had a statistically (p<0.005) higher mean BMI than grade 1/2 EEC patients.

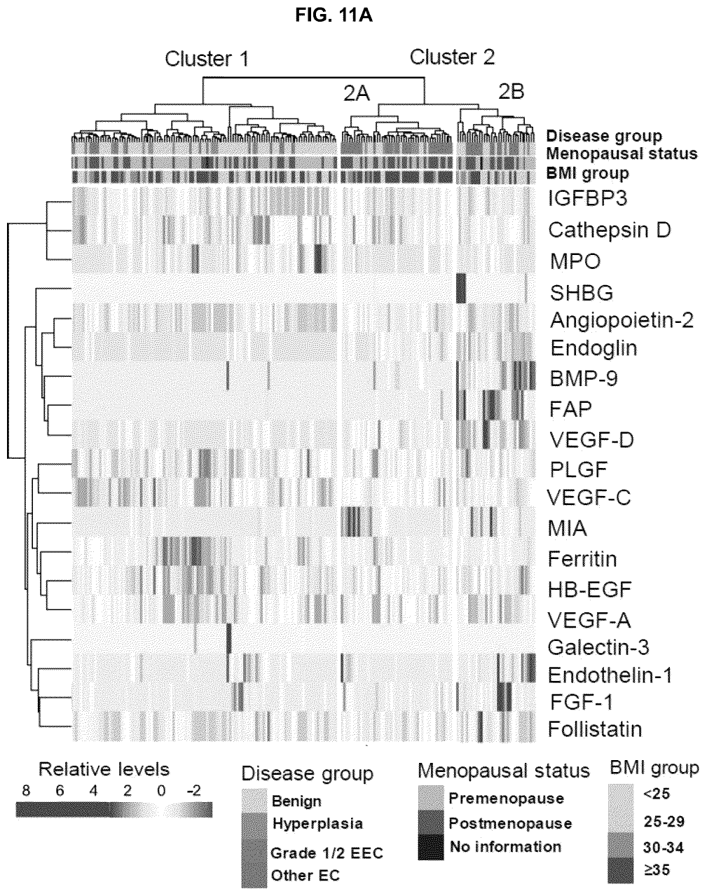

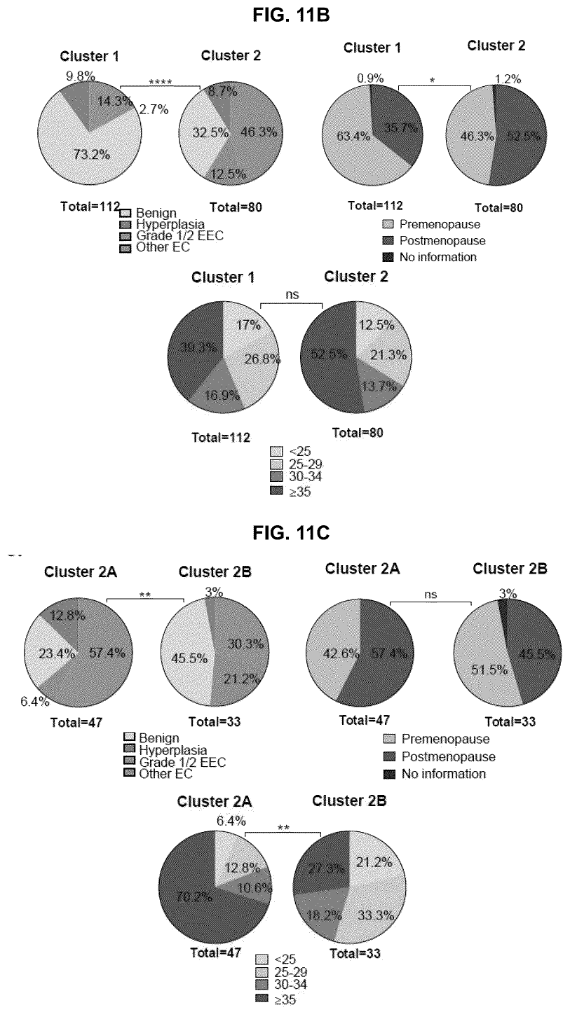

Global cervicovaginal protein profiles reveal distinct differences between endometrial cancer and benign patients. To visualize the cervicovaginal protein profiles of patients, an unsupervised hierarchical clustering analysis was conducted, and a heatmap with a dendrogram (FIG. 11A) displayed two separate patient clusters based on global protein levels. Overall, cluster one consisted of relatively low levels of the proteins, and cluster two contained relatively high levels of the proteins. To better understand the characteristics of patients belonging to these two distinct clusters, patient-related data such as disease group, BMI group, and menopausal status was overlaid and the statistical differences were analyzed in these characteristics between the two clusters. Disease group (FIG. 11B) and menopausal status significantly differed between the two clusters (p<0.0001 and p<0.05, respectively). However, the BMI groups did not statistically (p=0.34) vary between the two clusters. Briefly, cluster 1 predominantly consisted of benign (73.2%) and premenopausal (63.4%) patients. In contrast, cluster 2, consisted of mainly patients with EC (58.8%), and 52.5% of cluster 2 were postmenopausal patients. Further analysis of cluster 2 revealed two subclusters, 2A and 2B (FIG. 11C). Regarding relative protein levels, subcluster 2B exhibited higher levels of cervicovaginal proteins compared to subcluster 2A. When analyzed patient characteristics, subcluster 2A contained significantly (p=0.0097) more grade 1/2 EEC (57.4%) than subcluster 2B (30.3%), and subcluster 2B contained significantly more other EC subtypes (21.2%) than subcluster 2A (6.4%). Additionally, subcluster 2A included significantly (p=0.0013) more patients with a BMI of equal to or above 35 (70.2%) than subcluster 2B (27.3%). Overall, unsupervised hierarchical clustering revealed EC patients had altered global protein levels compared to benign patients.

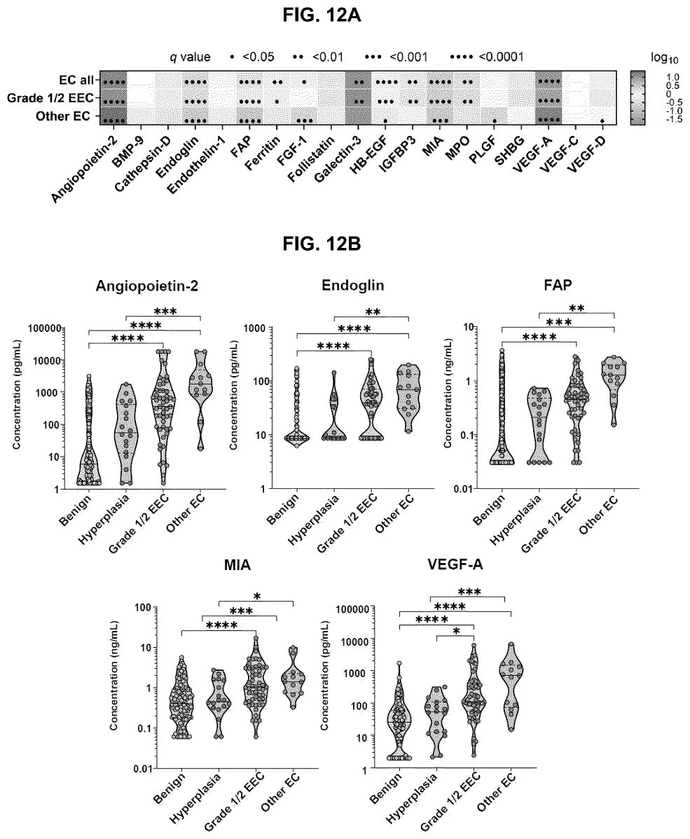

Cervicovaginal growth and angiogenic factor levels are increased with EC severity. To detect differences in protein levels between EC and benign patients, a fold change (FC) was calculated, and statistical differences were assessed. EC all, grade 1/2 EEC, and other EC were compared to benign patients (FIG. 12A) to identify individual proteins that significantly differed between these disease groups. When comparing EC all to benign patients, 11 out of 18 proteins were identified to be significantly different after FDR correction at 5%. Of these, eight proteins exhibited significant upregulation in EC all: angiopoietin-2 (q<0.0001), endoglin (q<0.0001), FAP (q<0.0001), ferritin (q=0.004), FGF-1 (q=0.02), MIA (q<0.0001), HB-EGF (q<0.0001), and VEGF-A (q<0.0001). In contrast, three proteins were significantly (q<0.01) downregulated in EC all, including galectin-3, MPO, and IGFBP3. When analyzing the differences between EC subtypes and benign patients, all previously mentioned proteins for EC all, except FGF-1, were also significantly altered in grade 1/2 EEC compared to benign patients.

Nine proteins were identified to be significantly (q ranging from 0.02 to <0.0001) upregulated in other EC types, including angiopoietin-2, endoglin, FAP, FGF-1, HB-EGF, MIA, PLGF, VEGF-A, and VEGF-D. Notably, angiopoietin-2, endoglin, FAP, and VEGF-A were significantly (q<0.0001) upregulated in all EC groups: EC all, grade 1/2 EEC, and other EC types. Furthermore, angiopoietin-2, endoglin, FAP, MIA, and VEGF-A exhibited higher FC in other EC types compared to grade 1/2 EEC (FIG. 12A). In the analysis of the protein levels across all the disease groups (FIG. 12B and FIG. 16), angiopoietin-2, endoglin, FAP, MIA, and VEGF-A levels were significantly (p<0.05-<0.001) higher in other EC patients compared to endometrial hyperplasia patients. Of those, only VEGF-A levels were statistically (p<0.05) elevated in grade 1/2 EEC compared to endometrial hyperplasia patients. Although not statistically significant, a noticeable trend was observed, in which the levels of angiopoietin-2, endoglin, FAP, MIA, and VEGF-A were increased with the severity of disease. These results highlighted some key proteins in CVLs that could be utilized to differentiate EC patients from benign patients.

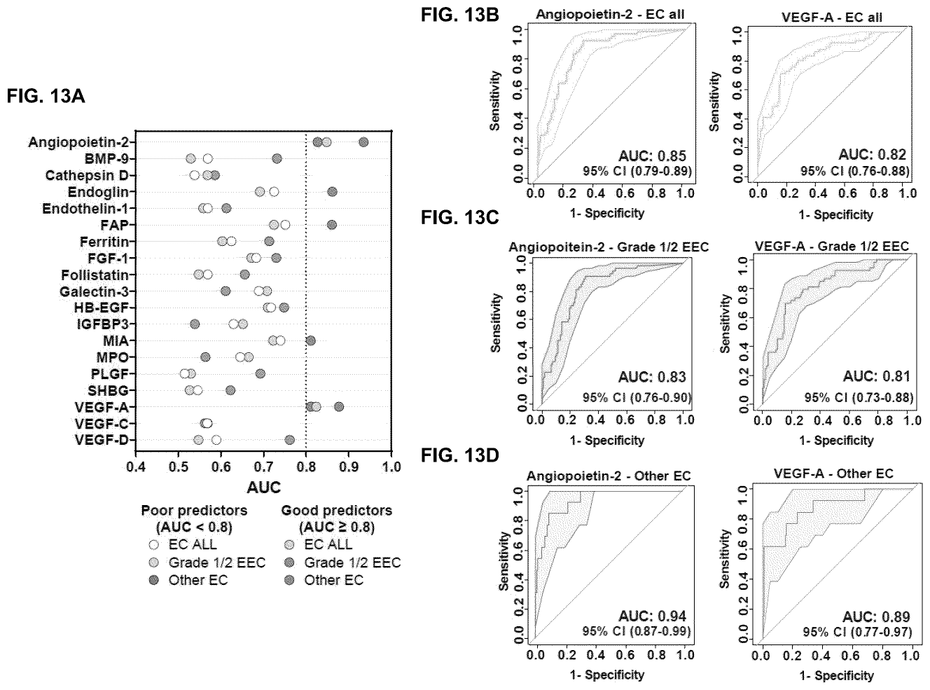

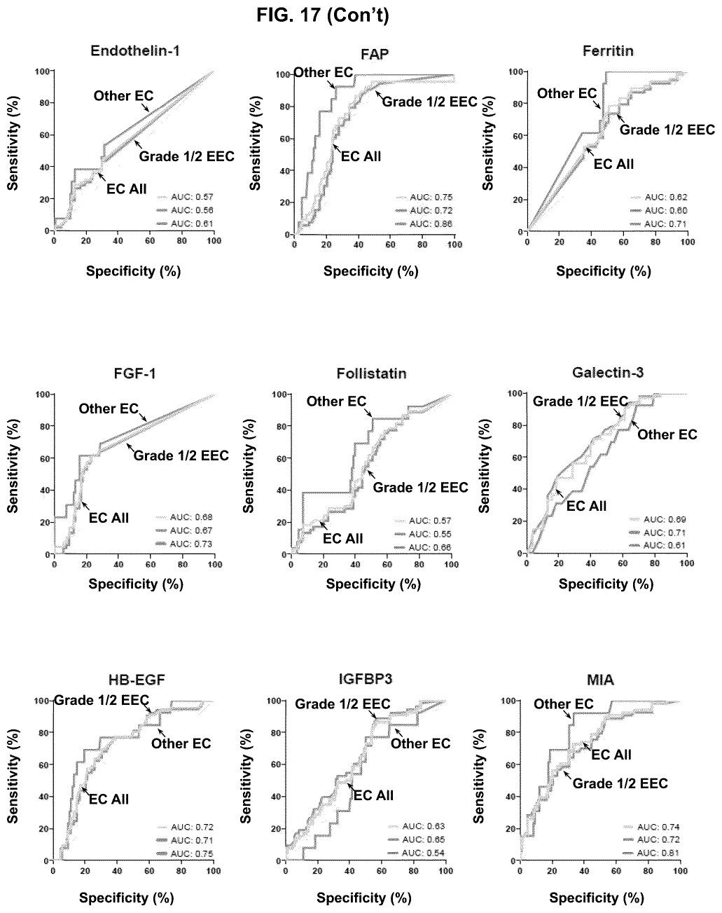

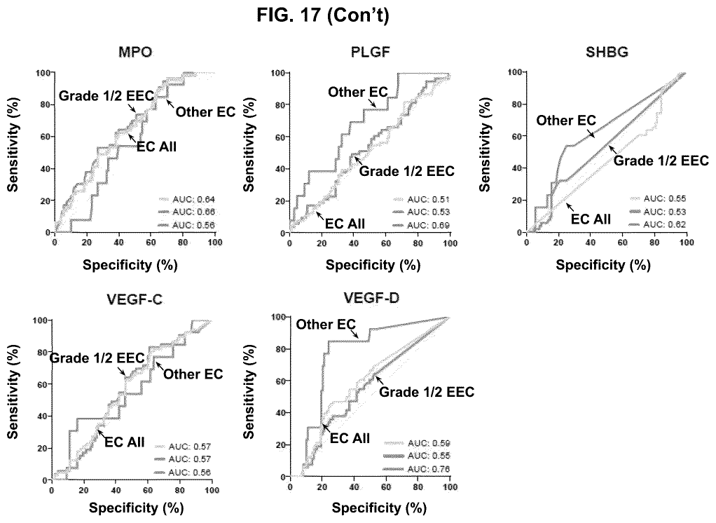

Growth and angiogenic markers exhibit biomarker potential in endometrial cancer. Next, univariate receiver operating characteristic (ROC) analysis was utilized to identify proteins exhibiting high sensitivity and specificity for EC all, grade 1/2 EEC, and other EC types compared to benign conditions. A ROC curve was plotted based on the sensitivity and specificity values of the proteins, and the area under the curve (AUC) was used to measure the strength of the discriminators. A protein with an AUC above or equal to 0.8 is considered a good discriminator, and a protein with an AUC above 0.9 is regarded as an excellent discriminator. Firstly, EC all patients to benign patients were compared and two proteins were identified as good discriminators for EC all: angiopoietin-2 and VEGF-A (FIG. 13A), with an AUC of 0.85 and 0.82, respectively (FIG. 13B). When comparing grade 1/2 EEC patients to benign patients, angiopoietin-2 and VEGF-A also exhibited a good discriminatory potential (FIG. 13C), with an AUC of 0.83 and 0.81, respectively. Finally, when examining the ROC for other EC types versus benign patients, four proteins were identified with a good discriminatory potential (FIG. 13D and FIG. 17): endoglin (AUC of 0.86), FAP (AUC of 0.86), MIA (AUC of 0.81), VEGF-A (AUC of 0.89) and one protein with an excellent discriminatory potential: angiopoietin-2 (AUC of 0.94) (FIG. 13D). Overall, univariate biomarker discovery analysis revealed several individual proteins with diagnostic potential for EC.

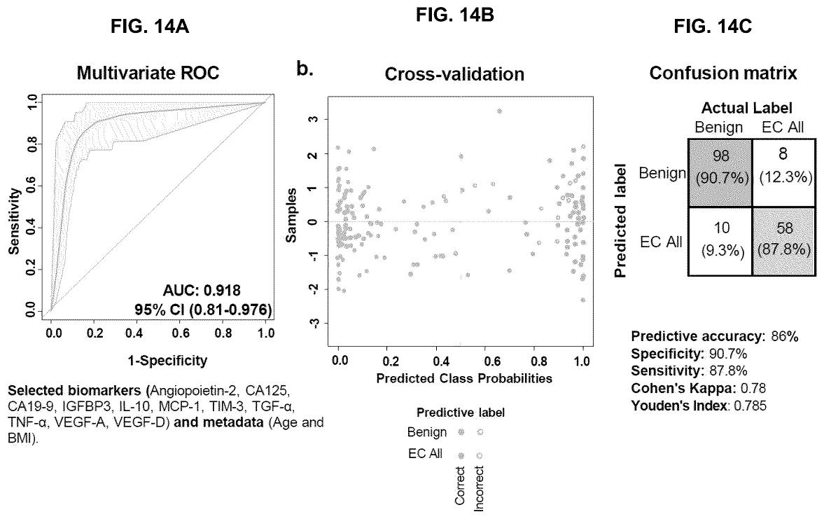

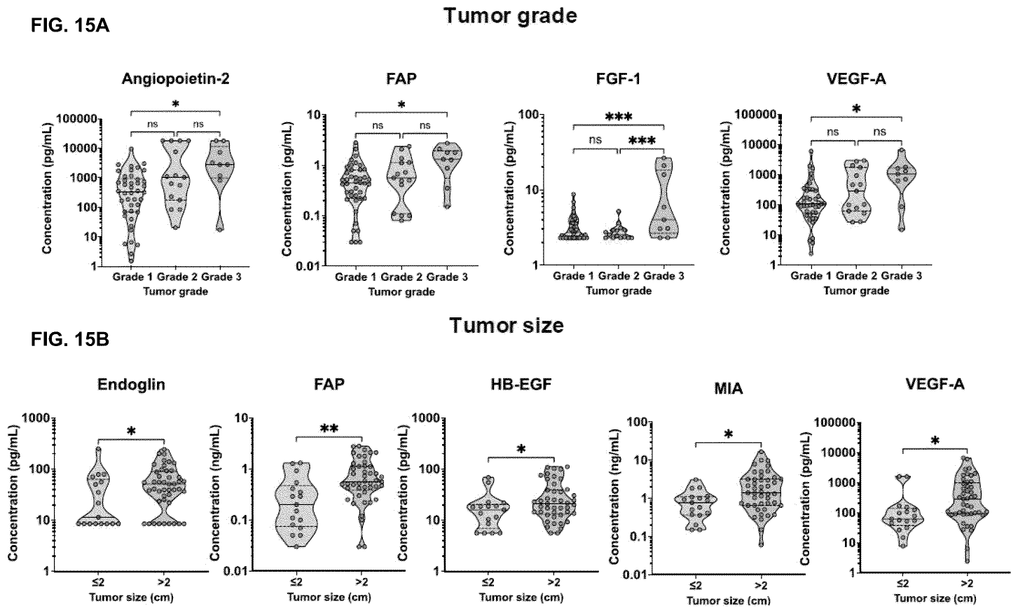

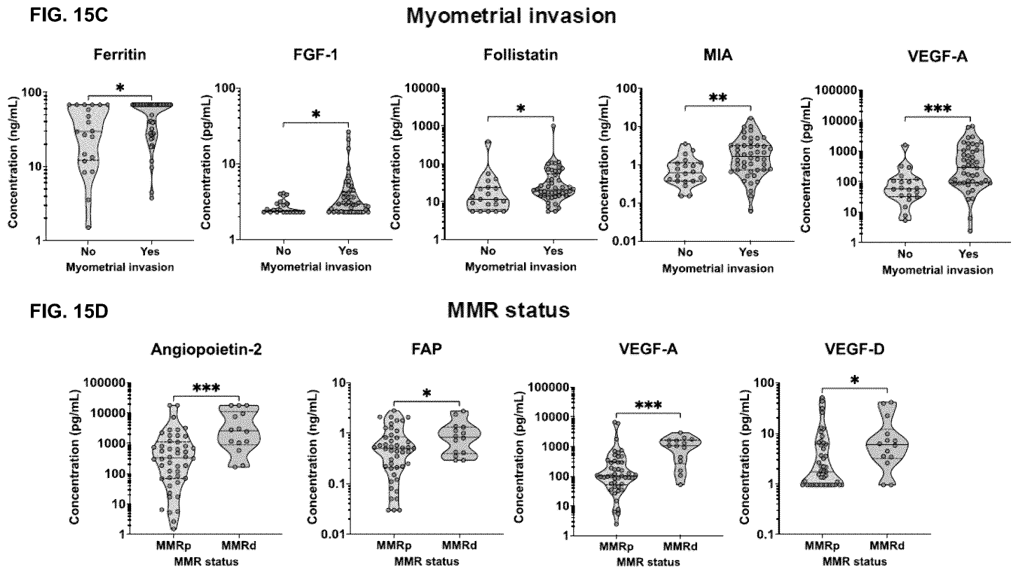

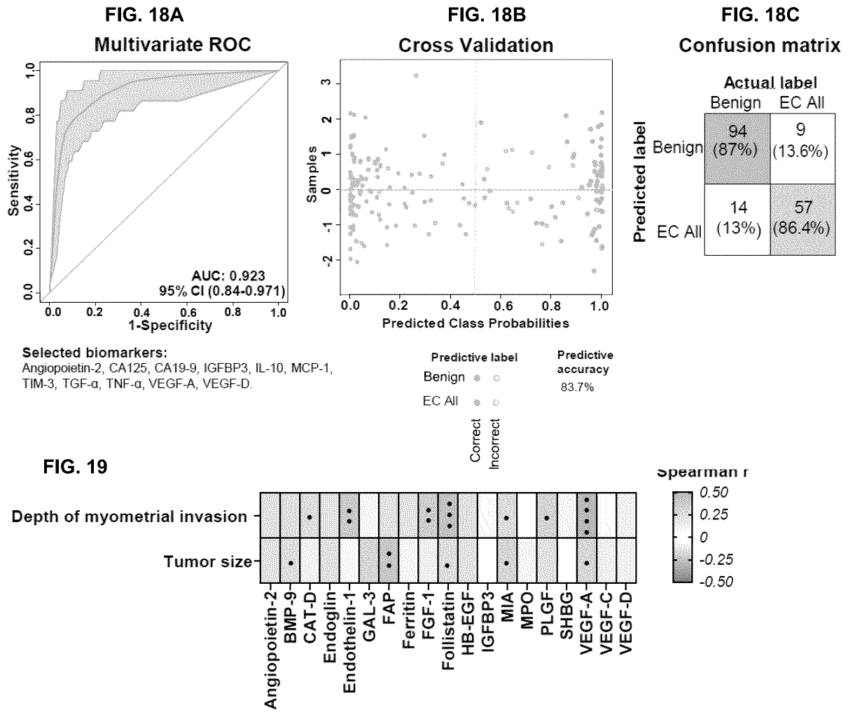

Multivariate protein model for EC diagnosis illustrates excellent discriminatory potential. Next, a multivariate approach was utilized and combinations of proteins were assessed to increase correct EC classification. Using multivariate ROC analysis, EC all patients were compared to benign patients using a logistic regression model (FIG. 14A). Proteins with high least absolute shrinkage and selection operator (LASSO) frequencies were selected and the model was refined through empirical testing of several protein combinations. Age and BMI were added to the model as they are known risk factors for EC. A combination of angiopoietin-2, IGFBP3 and VEGF-D were identified, with previously evaluated biomarkers as described in Example 1: carbohydrate antigen (CA)-125, carbohydrate antigen (CA)-19-9, interleukin (IL)-10, monocyte chemoattractant protein (MCP)-1, T cell immunoglobulin and mucin-domain containing (TIM)-3, transforming growth factor (TGF)-α, tumor necrosis factor (TNF)-α, VEGF-A. This biomarker and metadata combination generated an AUC of 0.918, which is indicative of an excellent discriminatory potential. Monte Carlo cross-validation was utilized to assess the predictive accuracy, and a confusion matrix showing correct and incorrect disease classification was generated. This combination of proteins demonstrated a predictive accuracy of 86% (FIG. 14B), with a specificity of 90.7% and a sensitivity of 87.8% (FIG. 14C). Overall, this multivariate protein model accurately classified 156 of 174 patients tested, including 98 of the 108 benign patients and 58 of the 66 EC patients. Cohen's kappa was calculated to be 0.78 which shows a substantial agreement between the actual label and the predictive label, and Youden's index of 0.785. After removing age and BMI from the protein model (FIGS. 18A, 18B, and 18C), the AUC of the model increased to 0.923; however, the predictive accuracy decreased to 83.7%. Furthermore, the specificity and sensitivity decreased to 87% and 86.4%, respectively. These analyses revealed that the multivariate protein model with patient metadata can accurately discriminate EC from benign conditions.

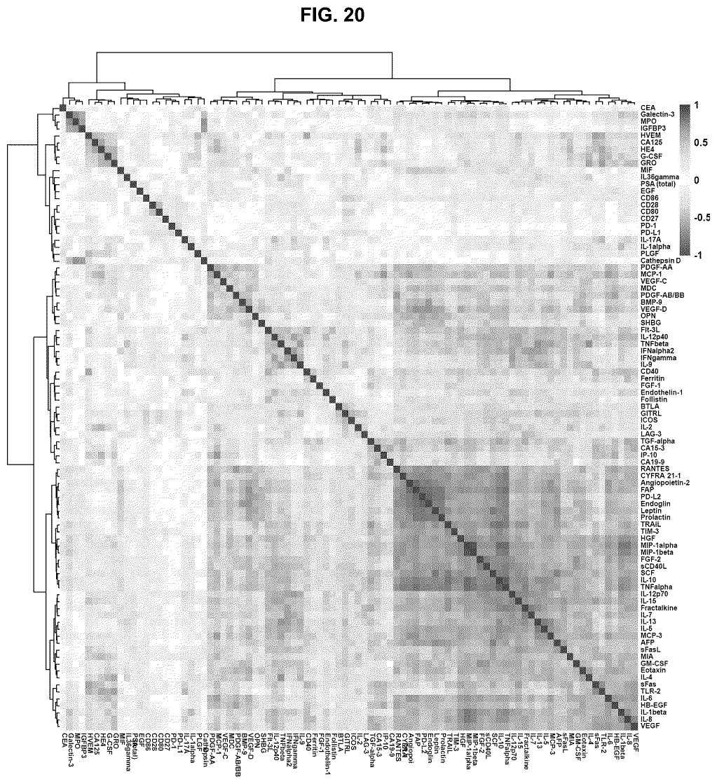

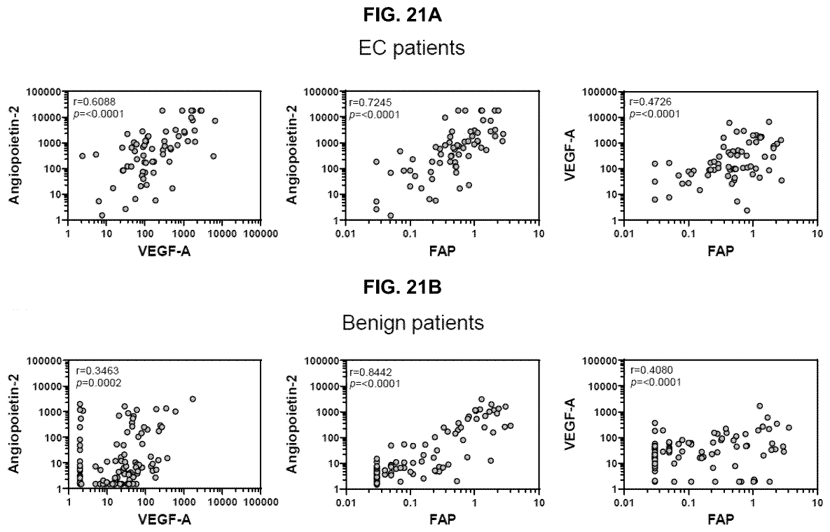

Cervicovaginal growth and angiogenic markers demonstrate prognostic utility in endometrial cancer. Lastly, to assess the prognostic utility of the proteins measured in CVLs, the relationship between protein levels and tumor characteristics was evaluated, such as tumor grade, tumor size, myometrial invasion, and mismatch repair (MMR) status. Data were also collected on the FIGO stage and the presence of lymphovascular invasion; however, these patient factors were unable to be analyzed due to the unbalanced distribution, with the majority of tumors being assigned FIGO stage I (87.1%) and having an absence of lymphovascular invasion (93.8%). When EC patients were classified based on tumor grade (FIG. 15A), significant elevation in the levels of angiopoietin-2 (p=0.015), FAP (p=0.033), FGF-1 (p=0.0004), and VEGF-A (p=0.018) were observed in the more advanced grade 3 tumors compared to grade 1 tumors. Interestingly, although not significant, a gradual increase in angiopoietin-2, FAP, and VEGF-A levels, as tumor grade increased. When stratifying EC patients based on tumor size (FIG. 15B), five proteins were significantly increased in patients with tumors larger than 2 cm: endoglin (p=0.03), FAP (p=0.001), HB-EGF (p=0.04), MIA (p=0.01), and VEGF-A (p=0.02), when compared to patients with smaller tumors (≤2 cm). Furthermore, through correlation analysis, concentrations of BMP-9, FAP, follistatin, MIA, and VEGF-A significantly (p ranging from 0.041 to 0.0012) correlate with tumor size (measured in cm) (FIG. 19). The protein levels between tumors with or without myometrial invasion were also assessed (FIG. 15C). The levels of five proteins: ferritin (p=0.03), FGF-1 (p=0.03), follistatin (p=0.04), MIA (p=0.0043), and VEGF-A (p=0.0009) were significantly higher in patients with tumors that invaded the myometrium compared to those with tumors that did not. When assessing the correlation of protein levels and myometrial invasion (measured in mm), six proteins: cathepsin-D, endothelin-1, FGF-1, follistatin, MIA, PLGF, and VEGF-A significantly (p ranging from 0.04 to <0.0001) correlated with the depth of myometrial invasion (FIG. 19). Only follistatin and MIA significantly (p ranging from 0.04 to <0.0001) correlated with tumor size and myometrial invasion depth. Patients with MMR deficient (MMRd) tumors exhibited significantly higher levels of angiopoietin-2 (p=0.0008), FAP (p=0.04), VEGF-A (p=0.0004), and VEGF-D (p=0.02) than patients with MMR proficient (MMRp) tumors (FIG. 15D). In summary, VEGF-A aligned with all the tumor characteristics evaluated, FAP was associated with tumor grade, size, and MMR status, and angiopoietin-2 was associated with tumor grade and MMR status. The growth and angiogenic markers were then correlated with a further 72 pro-and anti-inflammatory cytokines, chemokines, hormones, immune checkpoints and apoptosis-related factors described in Example 1 (FIG. 20). Analysis revealed strong positive correlations (correlation coefficient>0.7) among growth factors, pro-and anti-inflammatory cytokines, chemokines, hormones and immune checkpoint proteins, including proteins identified in the multivariate protein model such as Angiopoietin-2, IL-10, TNF-and TIM-3. Furthermore, when analyzing the correlation of angiopoietin 2, FAP, and VEGF concentrations in EC and benign patients (FIGS. 21A and 21B), these proteins significantly correlate with one another. This analysis illustrates the utility of proteins measured in the lower reproductive tract samples as diagnostic and prognostic markers for the upper reproductive tract disease, EC.

Unlike traditional diagnostic methods for EC, CVLs offer a minimally invasive approach due to the anatomical continuity of the female reproductive tract and hold promise for future EC diagnostics. The novel multivariate protein biomarker model described herein, in combination with innovative CVL sampling, may enable earlier diagnosis, as CVLs could be utilized in routine gynecological visits, thus facilitating earlier detection and intervention. This is particularly important considering rates of EC are increasing in younger females, hence, early detection and diagnosis could allow for fertility-sparing treatment options. While other detection methods such as serum-and tissue-based biomarkers have been assessed, their potential is limited by low sensitivity and specificity for EC.

In summary, the present invention illustrates the effectiveness of CVLs as a tool for EC detection. Angiopoietin-2, endoglin, FAP, MIA and VEGF-A were specifically identified as biomarkers with significant diagnostic and prognostic potential for EC. Through multivariate ROC analysis, an innovative biomarker model consisting of 11 growth and angiogenic markers in combination with patients' age and BMI displayed excellent discriminatory potential for EC, showcasing the diagnostic utility of CVL sampling. Specifically, the multivariate protein model accurately classified patients with a sensitivity of 87.8% and a specificity of 90.7%, a Cohen's Kappa of 0.78 and a Youden's index of 0.785. Moreover, these growth factors, detected in the CVLs, related to tumor characteristics, such as tumor grade, size, myometrial invasion, and MMR status, suggesting their prognostic potential. Overall, utilizing minimally invasive and less time-consuming sampling techniques such as CVLs could improve equity in access to early diagnosis, thereby enhancing patient outcomes and reducing health disparities in EC.

### EMBODIMENTS

The following embodiments are intended to be illustrative only and not to be limiting in any way.

Embodiment 1: A method comprising a) obtaining a biological sample from a patient, b) producing a profile of the biological sample collected in (a) by detecting at least five protein biomarkers selected from the group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3), and c) analyzing the biological sample profile produced in (b).

Embodiment 2: A method comprising a) obtaining a biological sample from a patient, b) producing a profile of the biological sample collected in (a) by detecting at least ten protein biomarkers selected from the group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3), and c) analyzing the biological sample profile produced in (b).

Embodiment 3: The method of embodiment 1 or embodiment 2, wherein the protein biomarkers further comprise TIM-3, IL-10, TRAIL, TGF-α, CYFRA 21-1, VEGF, TNFα, IL-6, SCF, fractalkine, IP-10, MCP-1, MCP-3, MIP-1α, MIP-1β, PDGF-AA, leptin, AFP, CA15-3, CD40, CA125, CA19-9, MDC, PD-L2, or a combination thereof.

Embodiment 4: The method of any one of embodiments 1-3, wherein the biological sample comprises a cervicovaginal lavage (CVL) sample, a urine sample, a vaginal swab or vaginal fluid, or cervicovaginal secretion, wherein the cervicovaginal secretion is collected via a physician, self-collected lavage or a menstrual cup.

Embodiment 5: The method of any one of embodiments 1-4, wherein the method predicts the risk of endometrial cancer in women.

Embodiment 6: The method of any one of embodiments 1-4, wherein the method diagnoses or prognoses endometrial cancer in women.

Embodiment 7: The method of any one of embodiments 1-6, wherein the endometrial cancer is EC type 1.

Embodiment 8: A method of diagnosing endometrial cancer (EC) in a subject in need thereof, the method comprising: a) obtaining a biological sample from the subject, b) producing a profile of the biological sample collected in (a) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3), and c) analyzing the biological sample profile produced in (b); wherein the subject is diagnosed with EC if the levels of at least five biomarkers are altered compared to a healthy control profile.

Embodiment 9: A method of diagnosing endometrial cancer (EC) in a subject in need thereof, the method comprising: a) obtaining a biological sample from the subject, b) producing a profile of the biological sample collected in a) by detecting at least ten protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3), and c) analyzing the biological sample profile produced in (b); wherein the subject is diagnosed with EC if the levels of at least ten biomarkers are altered compared to a healthy control profile.

Embodiment 10: The method of embodiment 8 or embodiment 9, wherein the subject is diagnosed with EC if the levels of angiopoietin-2, endoglin, FAP, ferritin, FGF-1, MIA, HB-EGF, or VEGF-A are elevated compared to the healthy control and the levels of galectin-3, MPO, or IGFBP-3 are decrease compared to the healthy control profile.

Embodiment 11: The method of any one of embodiments 8-10, wherein the protein biomarkers further comprise TIM-3, IL-10, TRAIL, TGF-α, CYFRA 21-1, VEGF, TNFα, IL-6, SCF, fractalkine, IP-10, MCP-1, MCP-3, MIP-1α, MIP-1β, PDGF-AA, leptin, AFP, CA15-3, CD40, CA125, CA19-9, MDC, PD-L2, or a combination thereof.

Embodiment 12: The method of any one of embodiments 8-11, wherein the biological sample comprises a cervicovaginal lavage (CVL) sample, a urine sample, a vaginal swab or vaginal fluid, or cervicovaginal secretion, wherein the cervicovaginal secretion is collected via a physician, self-collected lavage or a menstrual cup.

Embodiment 13: The method of any one of embodiments 8-12, wherein the method diagnoses EC type 1.

Embodiment 14: The method of any one of embodiments 8-13, wherein the healthy control profile is obtained from a healthy control subject.

Embodiment 15: A method of determining a prognosis of endometrial cancer (EC) in a subject in need thereof, the method comprising: a) obtaining a biological sample from the subject, b) producing a profile of the biological sample collected in (a) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3), and c) analyzing the biological sample profile produced in (b); wherein the subject has a poor prognosis if the levels of angiopoietin-2, endoglin, FAP, ferritin, FGF-1, MIA, HB-EGF, or VEGF-A are elevated compared to the healthy control and/or the levels of galectin-3, MPO, or IGFBP-3 are decrease compared to the healthy control profile.

Embodiment 16: A method of determining a prognosis of endometrial cancer (EC) in a subject in need thereof, the method comprising: a) obtaining a biological sample from the subject, b) producing a profile of the biological sample collected in (a) by detecting at least ten protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3), and c) analyzing the biological sample profile produced in (b); wherein the subject has a poor prognosis if the levels of angiopoietin-2, endoglin, FAP, ferritin, FGF-1, MIA, HB-EGF, or VEGF-A are elevated compared to the healthy control and/or the levels of galectin-3, MPO, or IGFBP-3 are decrease compared to the healthy control profile.

Embodiment 17: The method of embodiment 15 or embodiment 16, wherein the subject has a good prognosis if the levels of angiopoietin-2, endoglin, FAP, ferritin, FGF-1, MIA, HB-EGF, or VEGF-A are decreased compared to the healthy control and the levels of galectin-3, MPO, or IGFBP-3 are elevated compared to the healthy control profile.

Embodiment 18: The method of any one of embodiments 15-17, wherein the protein biomarkers further comprise TIM-3, IL-10, TRAIL, TGF-α, CYFRA 21-1, VEGF, TNFα, IL-6, SCF, fractalkine, IP-10, MCP-1, MCP-3, MIP-1α, MIP-1β, PDGF-AA, leptin, AFP, CA15-3, CD40, CA125, CA19-9, MDC, PD-L2, or a combination thereof.

Embodiment 19: The method of any one of embodiments 15-18, wherein the biological sample comprises a cervicovaginal lavage (CVL) sample, a urine sample, a vaginal swab or vaginal fluid, or cervicovaginal secretion, wherein the cervicovaginal secretion is collected via a physician, self-collected lavage or a menstrual cup.

Embodiment 20: The method of any one of embodiments 15-19, wherein the healthy control profile is obtained from a healthy control subject.

Embodiment 21: A method of treating endometrial cancer (EC) in a subject in need thereof, the method comprising: a) diagnosing the subject with EC by: i) obtaining a biological sample from the subject, ii) producing a profile of the biological sample collected in (i) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3); and iii) analyzing the biological sample profile produced in (ii); wherein the subject is diagnosed with EC if the levels of at least five biomarkers are altered compared to a healthy control profile; and b) administering an EC treatment to the subject and monitoring the therapy.

Embodiment 22: A method of treating endometrial cancer (EC) in a subject in need thereof, the method comprising: a) diagnosing the subject with EC by: i) obtaining a biological sample from the subject, ii) producing a profile of the biological sample collected in (i) by detecting at least ten protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3); and iii) analyzing the biological sample profile produced in (ii); wherein the subject is diagnosed with EC if the levels of at least ten biomarkers are altered compared to a healthy control profile; and b) administering an EC treatment to the subject and monitoring the therapy.

Embodiment 23: The method of embodiment 21 or embodiment 22, wherein the subject is diagnosed with EC if the levels of angiopoietin-2, endoglin, FAP, ferritin, FGF-1, MIA, HB-EGF, or VEGF-A are elevated compared to the healthy control and/or the levels of galectin-3, MPO, or IGFBP-3 are decrease compared to the healthy control profile.

Embodiment 24: The method of any one of embodiments 21-23, wherein the protein biomarkers further comprise TIM-3, IL-10, TRAIL, TGF-α, CYFRA 21-1, VEGF, TNFα, IL-6, SCF, fractalkine, IP-10, MCP-1, MCP-3, MIP-1α, MIP-1β, PDGF-AA, leptin, AFP, CA15-3, CD40, CA125, CA19-9, MDC, PD-L2, or a combination thereof.

Embodiment 25: The method of any one of embodiments 21-24, wherein the method diagnoses EC type 1.

Embodiment 26: The method of any one of embodiments 21-25, wherein the healthy control profile is obtained from a healthy control subject.

Embodiment 27: A method of monitoring a treatment for endometrial cancer (EC) in a subject in need thereof, the method comprising: a) obtaining a first biological sample from the subject; b) producing a baseline profile of the first biological sample collected in (a) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3); c) administering the treatment for EC to the subject; d) obtaining a second biological sample from the subject; e) producing a second profile of the second biological sample collected in (d) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3) and f) comparing the baseline profile of the first biological sample produced in (b) to the second profile of the second biological sample produced in (e); wherein the treatment is effective if the levels of at least five biomarkers are altered from the baseline profile as compared to the second profile.

Embodiment 28: A method of monitoring a treatment for endometrial cancer (EC) in a subject in need thereof, the method comprising: a) obtaining a first biological sample from the subject; b) producing a baseline profile of the first biological sample collected in (a) by detecting at least ten protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3); c) administering the treatment for EC to the subject; d) obtaining a second biological sample from the subject; e) producing a second profile of the second biological sample collected in (d) by detecting at least ten protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3) and f) comparing the baseline profile of the first biological sample produced in (b) to the second profile of the second biological sample produced in (e); wherein the treatment is effective if the levels of at least ten biomarkers are altered from the baseline profile as compared to the second profile.

Embodiment 29: The method of embodiment 27 or embodiment 28, wherein the treatment is effective if the levels of angiopoietin-2, endoglin, FAP, ferritin, FGF-1, MIA, HB-EGF, or VEGF-A are reduced from the baseline profile as compared to the second profile and/or the levels of galectin-3, MPO, or IGFBP-3 are elevated from the baseline profile as compared to the second profile.

Embodiment 30: The method of embodiment 27 or embodiment 28, wherein if the levels of angiopoietin-2, endoglin, FAP, ferritin, FGF-1, MIA, HB-EGF, or VEGF-A are elevated from the baseline profile as compared to the second profile and/or the levels of galectin-3, MPO, or IGFBP-3 are reduced from the baseline profile as compared to the second profile, the treatment administered to the subject is changed.

Embodiment 31: The method of any one of embodiments 27-30, wherein the protein biomarkers further comprise TIM-3, IL-10, TRAIL, TGF-α, CYFRA 21-1, VEGF, TNFα, IL-6, SCF, fractalkine, IP-10, MCP-1, MCP-3, MIP-α, MIP-1β, PDGF-AA, leptin, AFP, CA15-3, CD40, CA125, CA19-9, MDC, PD-L2, or a combination thereof.

Embodiment 32: The method of any one of embodiments 27-30, wherein the biological sample comprises a cervicovaginal lavage (CVL) sample, a urine sample, a vaginal swab or vaginal fluid, or cervicovaginal secretion, wherein the cervicovaginal secretion is collected via a physician, self-collected lavage or a menstrual cup.

Embodiment 33: An in vitro method of diagnosing endometrial cancer (EC) in a subject in need thereof, the method comprising: a) producing a profile from a biological sample obtained from a subject by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3); and b) analyzing the biological sample profile produced; wherein the subject is diagnosed with EC if the levels of at least five biomarkers are altered compared to a healthy control profile.

Embodiment 34: An in vitro method of diagnosing endometrial cancer (EC) in a subject in need thereof, the method comprising: a) producing a profile from a biological sample obtained from a subject by detecting at least ten protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3); and b) analyzing the biological sample profile produced; wherein the subject is diagnosed with EC if the levels of at least ten biomarkers are altered compared to a healthy control profile.

Embodiment 35: The method of embodiment 33 or embodiment 34, wherein the subject is diagnosed with EC if the levels of angiopoietin-2, endoglin, FAP, ferritin, FGF-1, MIA, HB-EGF, or VEGF-A are elevated compared to the healthy control and/or the levels of galectin-3, MPO, or IGFBP-3 are decrease compared to the healthy control profile.

Embodiment 36: The method of any one of embodiments 33-35, wherein the protein biomarkers further comprise TIM-3, IL-10, TRAIL, TGF-α, CYFRA 21-1, VEGF, TNFα, IL-6, SCF, fractalkine, IP-10, MCP-1, MCP-3, MIP-1α, MIP-1β, PDGF-AA, leptin, AFP, CA15-3, CD40, CA125, CA19-9, MDC, PD-L2, or a combination thereof.

Embodiment 37: The method of any one of embodiments 33-36, wherein the biological sample comprises a cervicovaginal lavage (CVL) sample, a urine sample, a vaginal swab or vaginal fluid, or cervicovaginal secretion, wherein the cervicovaginal secretion is collected via a physician, self-collected lavage or a menstrual cup.

Embodiment 38: The method of any one of embodiments 33-37, wherein the endometrial cancer is EC type 1.

Embodiment 39: The method of any one of embodiments 33-38, wherein the healthy control profile is obtained from a healthy control subject.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. An in vitro method comprising:
a) obtaining a biological sample from a patient;
b) producing a profile of the biological sample collected in (a) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3); and
c) analyzing the biological sample profile produced in (b).

2. The method of claim 1, wherein the protein biomarkers further comprise TIM-3, IL-10, TRAIL, TGF-α, CYFRA 21-1, VEGF, TNFα, IL-6, SCF, fractalkine, IP-10, MCP-1, MCP-3, MIP-1α, MIP-1β, PDGF-AA, leptin, AFP, CA15-3, CD40, CA125, CA19-9, MDC, PD-L2, or a combination thereof.

3. The method of claim 1, wherein producing the profile comprises detecting at least ten protein biomarkers; wherein the protein biomarkers are selected from the group consisting of angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3).

4. The method of claim 1, wherein the biological sample comprises a cervicovaginal lavage (CVL) sample, a urine sample, a vaginal swab or vaginal fluid, or cervicovaginal secretion, wherein the cervicovaginal secretion is collected via a physician, self-collected lavage or a menstrual cup.

5. The method of claim 1, wherein the method predicts the risk of endometrial cancer in women.

6. The method of claim 1, wherein the method diagnoses endometrial cancer in women.

7. A method of diagnosing endometrial cancer (EC) in a subject in need thereof, the method comprising;
a) obtaining a biological sample from the subject;
b) producing a profile of the biological sample collected in (a) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3); and
c) analyzing the biological sample profile produced in (b);
wherein the subject is diagnosed with EC if the levels of at least five biomarkers are altered compared to a healthy control profile.

8. The method of claim 7, wherein the subject is diagnosed with EC if the levels of angiopoietin-2, endoglin, FAP, ferritin, FGF-1, MIA, HB-EGF, or VEGF-A are elevated compared to the healthy control and the levels of galectin-3, MPO, and IGFBP-3 are decrease compared to the healthy control profile.

9. The method of claim 7, wherein producing the profile comprises detecting at least ten protein biomarkers; wherein the protein biomarkers are selected from the group consisting of angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3).

10. The method of claim 7, wherein the protein biomarkers further comprise TIM-3, IL-10, TRAIL, TGF-α, CYFRA 21-1, VEGF, TNFα, IL-6, SCF, fractalkine, IP-10, MCP-1, MCP-3, MIP-1α, MIP-1β, PDGF-AA, leptin, AFP, CA15-3, CD40, CA125, CA19-9, MDC, PD-L2, or a combination thereof.

11. The method of claim 7, wherein the biological sample comprises a cervicovaginal lavage (CVL) sample, a urine sample, a vaginal swab or vaginal fluid, or cervicovaginal secretion, wherein the cervicovaginal secretion is collected via a physician, self-collected lavage or a menstrual cup.

12. The method of claim 7, wherein the method diagnoses EC type 1.

13. The method of claim 7, wherein the healthy control profile is obtained from a healthy control subject.

14. The method of claim 7 further comprising administering an EC treatment to the subject and monitoring the therapy.

15. A method of monitoring a treatment for endometrial cancer (EC) in a subject in need thereof, the method comprising;
a) obtaining a first biological sample from the subject;
b) producing a baseline profile of the first biological sample collected in (a) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3);
c) administering the treatment for EC to the subject;
d) obtaining a second biological sample from the subject;
e) producing a second profile of the second biological sample collected in (d) by detecting at least five protein biomarkers selected from a group comprising angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3); and
f) comparing the baseline profile of the first biological sample produced in (b) to the second profile of the second biological sample produced in (e);
wherein the treatment is effective if the levels of at least five biomarkers are altered from the baseline profile as compared to the second profile.

16. The method of claim 15, wherein the treatment is effective if the levels of angiopoietin-2, endoglin, FAP, ferritin, FGF-1, MIA, HB-EGF, or VEGF-A are reduced from the baseline profile as compared to the second profile and/or the levels of galectin-3, MPO, or IGFBP-3 are elevated from the baseline profile as compared to the second profile.

17. The method of claim 15, wherein if the levels of angiopoietin-2, endoglin, FAP, ferritin, FGF-1, MIA, HB-EGF, or VEGF-A are elevated from the baseline profile as compared to the second profile and/or the levels of galectin-3, MPO, or IGFBP-3 are reduced from the baseline profile as compared to the second profile, the treatment administered to the subject is changed.

18. The method of claim 15, wherein producing the profile comprises detecting at least ten protein biomarkers; wherein the protein biomarkers are selected from the group consisting of angiopoietin-2, endoglin, fibroblast activation protein (FAP), ferritin, fibroblast growth factor 1 (FGF-1), melanoma inhibitory activity (MIA) protein, heparin-binding EGF-like growth factor (HB-EGF), vascular endothelial growth factor A (VEGF-A), galectin-3, myeloperoxidase (MPO), and insulin-like growth factor binding protein 3 (IGFBP-3).

19. The method of claim 15, wherein the protein biomarkers further comprise TIM-3, IL-10, TRAIL, TGF-α, CYFRA 21-1, VEGF, TNFα, IL-6, SCF, fractalkine, IP-10, MCP-1, MCP-3, MIP-1α, MIP-1β, PDGF-AA, leptin, AFP, CA15-3, CD40, CA125, CA19-9, MDC, PD-L2, or a combination thereof.

20. The method of claim 15, wherein the biological sample comprises a cervicovaginal lavage (CVL) sample, a urine sample, a vaginal swab or vaginal fluid, or cervicovaginal secretion, wherein the cervicovaginal secretion is collected via a physician, self-collected lavage or a menstrual cup.
